SEQUENCE LISTING (23 sequences)

SEQ ID NO: 1 - protein, 762 aa, synthetic construct
  source 1..762: note = TFRC_G4S_IFN-a2a mRNA
ATGATGGTGGACGGCGACAACAGCCACGTGGAAATGAAGCTGGCCGTGGACGAGGAAGAGAACGCCGACAACAACACCAAGGCCAACGTGACCAAGCCTAAGAGATGCAGCGGCAGCATCTGCTACGGCACAATCGCCGTGATCGTGTTCTTCCTGATCGGCTTTATGATCGGCTACCTGGGCTACTGCAAGAGCAGTGATGGACCTGGCGAAACAGGCGGAGGCGGAGGATCTGGTGGCGGAGGAAGCGGTGGCGGCGGATCTTGTGATCTGCCTCAGACACACAGCCTGGGCAGCAGACGAACACTGATGCTGCTGGCCCAGATGCGGAAGATCAGCCTGTTCAGCTGCCTGAAGGACCGGCACGATTTCGGCTTCCCTCAAGAGGAATTCGGCAACCAGTTCCAGAAGGCCGAGACAATCCCTGTGCTGCACGAGATGATCCAGCAGATCTTCAACCTGTTCTCCACCAAGGACAGCAGCGCCGCCTGGGATGAGACACTGCTGGACAAGTTCTACACCGAGCTGTACCAGCAGCTGAATGACCTGGAAGCCTGCGTGATCCAAGGCGTGGGAGTGACAGAGACACCCCTGATGAAGGAAGATAGCATCCTGGCCGTGCGCAAGTACTTCCAGCGGATCACCCTGTACCTGAAAGAGAAGAAGTACAGCCCCTGCGCCTGGGAAGTCGTGCGGGCCGAAATCATGAGAAGCTTCAGCCTGAGCACCAACCTGCAAGAGAGCCTGCGGAGCAAAGAGTGA

SEQ ID NO: 2 - protein, 1836 aa, synthetic construct
  source 1..1836: note = TFRC_G4S_IL-12 mRNA
ATGATGGTGGACGGCGACAACAGCCACGTGGAAATGAAGCTGGCCGTGGACGAGGAAGAGAACGCCGACAACAACACCAAGGCCAACGTGACCAAGCCTAAGAGATGCAGCGGCAGCATCTGCTACGGCACAATCGCCGTGATCGTGTTCTTCCTGATCGGCTTTATGATCGGCTACCTGGGCTACTGCAAGAGCAGTGATGGACCTGGCGAAACAGGCGGAGGCGGAGGATCTGGTGGCGGAGGAAGTGGCGGCGGAGGTTCTATTTGGGAGCTGAAGAAAGACGTGTACGTGGTGGAACTGGACTGGTATCCCGATGCTCCTGGCGAGATGGTGGTGCTGACCTGCGATACCCCTGAAGAGGACGGCATCACCTGGACACTGGATCAGTCTAGCGAGGTGCTCGGCAGCGGCAAGACCCTGACCATCCAAGTGAAAGAGTTTGGCGACGCCGGCCAGTACACCTGTCACAAAGGCGGAGAAGTGCTGAGCCACAGCCTGCTGCTGCTCCACAAGAAAGAGGATGGCATTTGGAGCACCGACATCCTGAAGGACCAGAAAGAGCCCAAGAACAAGACCTTCCTGAGATGCGAGGCCAAGAACTACAGCGGCCGGTTCACATGTTGGTGGCTGACCACCATCAGCACCGACCTGACCTTCAGCGTGAAGTCCAGCAGAGGCAGCAGTGATCCTCAGGGCGTTACATGTGGCGCCGCTACACTGTCTGCCGAAAGAGTGCGGGGCGATAACAAAGAATACGAGTACAGCGTGGAATGCCAAGAGGACAGCGCCTGTCCAGCCGCCGAAGAGTCTCTGCCTATCGAAGTGATGGTCGACGCCGTGCACAAGCTGAAGTACGAGAACTACACCAGCAGCTTTTTCATCCGGGACATCATCAAGCCCGATCCTCCAAAGAACCTGCAGCTGAAGCCTCTGAAGAACAGCAGACAGGTGGAAGTGTCCTGGGAGTACCCCGACACCTGGTCTACACCCCACAGCTACTTCAGCCTGACCTTTTGCGTGCAAGTGCAGGGCAAGTCCAAGCGCGAGAAAAAGGACCGGGTGTTCACCGACAAGACCAGCGCCACCGTGATCTGCAGAAAGAACGCCAGCATCAGCGTCAGAGCCCAGGACCGGTACTACAGCAGCTCTTGGAGCGAATGGGCCAGCGTGCCATGTAGCGGAGGTGGTGGTAGCGGAGGCGGCGGAAGCGGCGGTGGTGGATCAGGTGGTGGTGGCTCTAGAAACCTGCCAGTGGCTACCCCTGATCCTGGCATGTTCCCTTGTCTGCACCACAGCCAGAACCTGCTGAGAGCCGTGTCCAACATGCTGCAGAAGGCCAGACAGACCCTGGAATTCTACCCCTGCACCAGCGAGGAAATCGACCACGAGGACATCACCAAGGATAAGACCAGCACCGTGGAAGCCTGCCTGCCTCTGGAACTGACCAAGAACGAGAGCTGCCTGAACAGCCGGGAAACCTCCTTCATCACCAACGGCTCTTGCCTGGCCAGCAGAAAGACAAGCTTCATGATGGCCCTGTGCCTGAGCAGCATCTACGAGGACCTGAAGATGTACCAGGTGGAATTCAAGACCATGAACGCCAAGCTGCTGATGGACCCCAAGCGGCAGATCTTCCTGGACCAGAACATGCTGGCTGTGATCGACGAGCTGATGCAGGCCCTGAACTTCAACAGCGAGACAGTGCCCCAGAAGTCTAGCCTGGAAGAACCCGACTTCTACAAGACCAAGATCAAGCTGTGCATCCTGCTGCACGCCTTCCGGATCAGAGCCGTGACCATCGACAGAGTGATGAGCTACCTGAACGCCTCCTGA

SEQ ID NO: 3 - protein, 15 aa, synthetic construct
  source 1..15: note = G4S Linker
GGGGSGGGGSGGGGS

SEQ ID NO: 4 - protein, 15 aa, synthetic construct
  source 1..15: note = EAAAK linker
EAAAKEAAAKEAAAK

SEQ ID NO: 5 - protein, 5 aa, synthetic construct
  source 1..5: note = G4S Linker
  REGION 1..5: note = The sequence can comprise 1-100 repeats of the GGGGS subsequence
GGGGS

SEQ ID NO: 6 - protein, 5 aa, synthetic construct
  source 1..5: note = EAAAK linker
  REGION 1..5: note = The entire amino acid sequence can be repeated 1-100 times
EAAAK

SEQ ID NO: 7 - protein, 220 aa, synthetic construct
  source 1..220: note = TFRC_G4S_IL-2
MMVDGDNSHVEMKLAVDEEENADNNTKANVTKPKRCSGSICYGTIAVIVFFLIGFMIGYLGYCKSSDGPGETGGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTL

SEQ ID NO: 8 - protein, 191 aa, synthetic construct
  source 1..191: note = FasL_G4S_IL-2
MLKKRGNHSTGLCLLVMFFMVLVALVGLGLGMFQLFHLQKETGGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 9 - protein, 611 aa, synthetic construct
  source 1..611: note = TFRC_G4S_IL-12
MMVDGDNSHVEMKLAVDEEENADNNTKANVTKPKRCSGSICYGTIAVIVFFLIGFMIGYLGYCKSSDGPGETGGGGGSGGGGSGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 10 - protein, 253 aa, synthetic construct
  source 1..253: note = TFRC_G4S_IFN-a2a
MMVDGDNSHVEMKLAVDEEENADNNTKANVTKPKRCSGSICYGTIAVIVFFLIGFMIGYLGYCKSSDGPGETGGGGGSGGGGSGGGGSCDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 11 - protein, 73 aa, synthetic construct
  source 1..73: note = TFRC domain
MMVDGDNSHVEMKLAVDEEENADNNTKANVTKPKRCSGSICYGTIAVIVFFLIGFMIGYLGYCKSSDGPGETG

SEQ ID NO: 12 - protein, 43 aa, synthetic construct
  source 1..43: note = FasL domain
MLKKRGNHSTGLCLLVMFFMVLVALVGLGLGMFQLFHLQKETG

SEQ ID NO: 13 - protein, 723 aa, synthetic construct
  source 1..723: note = membrane-bound IL-7 mRNA
ATGATGGTGGACGGCGACAACAGCCACGTGGAGATGAAGCTGGCCGTGGACGAGGAGGAGAACGCCGACAACAACACCAAGGCCAACGTGACCAAGCCCAAGCGCTGCAGCGGCAGCATCTGCTACGGCACCATCGCCGTGATCGTGTTCTTCCTGATCGGCTTCATGATCGGCTACCTGGGCTACTGCAAGAGCAGCGACGGCCCCGGCGAGACCGGCGGAGGCGGCGGAAGCGGCGGCGGAGGCAGCGGCGGAGGCGGAAGCGACTGCGACATCGAGGGCAAGGACGGCAAGCAGTACGAGAGCGTGCTGATGGTGAGCATCGACCAGCTGCTGGACAGCATGAAGGAGATCGGCAGCAACTGCCTGAACAACGAGTTCAACTTCTTCAAGCGCCACATCTGCGACGCCAACAAGGAGGGCATGTTCCTGTTCCGCGCCGCCCGCAAGCTGCGCCAGTTCCTGAAGATGAACAGCACCGGCGACTTCGACCTGCACCTGCTGAAGGTGAGCGAGGGCACCACCATCCTGCTGAACTGCACCGGCCAGGTGAAGGGCCGCAAGCCCGCCGCCCTGGGCGAGGCCCAGCCCACCAAGAGCCTGGAGGAGAACAAGAGCCTGAAGGAGCAGAAGAAGCTGAACGACCTGTGCTTCCTGAAGCGCCTGCTGCAGGAGATCAAGACCTGCTGGAACAAGATCCTGATGGGCACCAAGGAGCACTAA

SEQ ID NO: 14 - protein, 158 aa, synthetic construct
  source 1..158: note = HPV-16 E6 UniProt: P03126
MHQKRTAMFQDPQERPRKLPQLCTELQTTIHDIILECVYCKQQLLRREVYDFAFRDLCIVYRDGNPYAVCDKCLKFYSKISEYRHYCYSLYGTTLEQQYNKPLCDLLIRCINCQKPLCPEEKQRHLDKKQRFHNIRGRWTGRCMSCCRSSRTRRETQL

SEQ ID NO: 15 - protein, 98 aa, synthetic construct
  source 1..98: note = HPV-16 E7 UniProt: P03129
MHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKP

SEQ ID NO: 16 - protein, 158 aa, synthetic construct
  source 1..158: note = HPV-18 E6 UniProt: P06463
MARFEDPTRRPYKLPDLCTELNTSLQDIEITCVYCKTVLELTEVFEFAFKDLFVVYRDSIPHAACHKCIDFYSRIRELRHYSDSVYGDTLEKLTNTGLYNLLIRCLRCQKPLNPAEKLRHLNEKRRFHNIAGHYRGQCHSCCNRARQERLQRRRETQV

SEQ ID NO: 17 - protein, 105 aa, synthetic construct
  source 1..105: note = HPV-18 E7 UniProt: P06788
MHGPKATLQDIVLHLEPQNEIPVDLLCHEQLSDSEEENDEIDGVNHQHLPARRAEPQRHTMLCMCCKCEARIKLVVESSADDLRAFQQLFLNTLSFVCPWCASQQ

SEQ ID NO: 18 - protein, 701 aa, synthetic construct
  source 1..701: note = E6 mRNA
GGAAAUAAGAGAGAAAAGAAGAGUAAGAAGAAAUAUAAGAGCCACCAUGCACCAGAAACGGACCGCCAUGUUCCAGGAUCCUCAAGAGAGGCCCAGAAAGCUGCCUCAGCUGUGUACCGAGCUGCAGACCACCAUCCACGACAUCAUCCUGGAAUGCGUGUACUGCAAGCAGCAGCUCCUGCGGAGAGAGGUGUACGAUUUCGCCUUCCGGGACCUGUGCAUCGUGUACAGAGAUGGCAACCCCUACGCCGUGUGCGACAAGUGCCUGAAGUUCUACAGCAAGAUCAGCGAGUACCGGCACUACUGCUACAGCCUGUACGGCACCACACUGGAACAGCAGUACAACAAGCCCCUGUGCGACCUGCUGAUCCGGUGCAUCAACUGCCAGAAACCUCUGUGCCCCGAGGAAAAGCAGCGGCACCUGGACAAGAAGCAGCGGUUCCACAACAUCAGAGGCCGGUGGACCGGCAGAUGCAUGAGCUGUUGUCGGAGCAGCAGAACCAGACGGGAAACCCAGCUGUGAGCUGCCUUCUGCGGGGCUUGCCUUCUGGCCAUGCCCUUCUUCUCUCCCUUGCACCUGUACCUCUUGGUCUUUGAAUAAAGCCUGAGUAGGAAGUCUAGAAAAAAAAAAAAAAAAAAAAAAAAAAAAAAAAAAAAAAAAAAAAAAAAAAAAAAAAAAAAAAAAAAAAAAAAAAAAAAAA

SEQ ID NO: 19 - protein, 521 aa, synthetic construct
  source 1..521: note = E7 mRNA
GGAAAUAAGAGAGAAAAGAAGAGUAAGAAGAAAUAUAAGAGCCACCAUGCACGGCGAUACCCCUACACUGCACGAGUACAUGCUGGACCUGCAGCCUGAGACAACCGACCUGUACUGCUACGAGCAGCUGAACGACAGCAGCGAGGAAGAGGACGAGAUUGACGGACCUGCCGGACAGGCCGAACCUGAUAGAGCCCACUACAAUAUCGUGACCUUCUGCUGCAAGUGCGACAGCACCCUGAGACUGUGUGUGCAGAGCACCCACGUGGACAUCAGAACCCUGGAAGAUCUGCUGAUGGGCACCCUGGGCAUCGUGUGCCCUAUCUGUAGCCAGAAGCCUUGAGCUGCCUUCUGCGGGGCUUGCCUUCUGGCCAUGCCCUUCUUCUCUCCCUUGCACCUGUACCUCUUGGUCUUUGAAUAAAGCCUGAGUAGGAAGUCUAGAAAAAAAAAAAAAAAAAAAAAAAAAAAAAAAAAAAAAAAAAAAAAAAAAAAAAAAAAAAAAAAAAAAAAAAAAAAAAAAA

SEQ ID NO: 20 - protein, 1214 aa, synthetic construct
  source 1..1214: note = CD86 mRNA
GGAAAUAAGAGAGAAAAGAAGAGUAAGAAGAAAUAUAAGAGCCACCAUGGAUCCCCAGUGUACCAUGGGCCUGAGCAACAUCCUGUUCGUGAUGGCCUUUCUGCUGAGCGGAGCCGCUCCUCUGAAGAUCCAGGCCUACUUCAACGAGACAGCCGACCUGCCUUGCCAGUUCGCCAACAGCCAGAAUCAGAGCCUGAGCGAGCUGGUGGUGUUCUGGCAGGACCAAGAGAACCUGGUGCUGAACGAGGUGUACCUGGGCAAAGAAAAGUUCGACAGCGUGCACAGCAAGUACAUGGGCAGAACCAGCUUCGACUCCGACAGCUGGACACUGAGACUGCACAACCUGCAGAUCAAGGACAAGGGCCUGUACCAGUGCAUCAUCCACCACAAGAAACCCACCGGCAUGAUCAGAAUCCACCAGAUGAACAGCGAGCUGAGCGUGCUGGCCAACUUCAGCCAGCCUGAGAUCGUGCCCAUCAGCAACAUCACCGAGAACGUGUACAUCAACCUGACCUGCAGCAGCAUCCACGGCUACCCCGAGCCUAAGAAAAUGUCCGUGCUGCUGCGGACCAAGAACAGCACCAUCGAGUACGACGGCGUGAUGCAGAAAAGCCAGGACAACGUGACCGAGCUGUACGACGUGUCCAUCAGCCUGUCCGUGUCAUUCCCCGACGUGACCAGCAAUAUGACCAUCUUCUGCAUCCUGGAAACCGACAAGACCCGGCUGCUGUCUAGCCCCUUCAGCAUCGAGCUGGAAGAUCCCCAGCCUCCUCCUGAUCACAUCCCUUGGAUUACCGCCGUGCUGCCCACCGUGAUCAUCUGCGUGAUGGUGUUUUGCCUGAUCCUGUGGAAGUGGAAGAAGAAGAAACGGCCUCGGAACAGCUACAAGUGCGGCACCAACACCAUGGAACGGGAAGAGAGCGAGCAGACCAAGAAGAGAGAGAAGAUUCACAUCCCCGAGCGGAGCGACGAAGCCCAGAGAGUGUUUAAGAGCAGCAAGACCAGCAGCUGCGACAAGAGCGAUACCUGCUUCUGAGCUGCCUUCUGCGGGGCUUGCCUUCUGGCCAUGCCCUUCUUCUCUCCCUUGCACCUGUACCUCUUGGUCUUUGAAUAAAGCCUGAGUAGGAAGUCUAGAAAAAAAAAAAAAAAAAAAAAAAAAAAAAAAAAAAAAAAAAAAAAAAAAAAAAAAAAAAAAAAAAAAAAAAAAAAAAAAA

SEQ ID NO: 21 - protein, 890 aa, synthetic construct
  source 1..890: note = membrane-bound IL-2 mRNA
GGAAAUAAGAGAGAAAAGAAGAGUAAGAAGAAAUAUAAGAGCCACCAUGAUGGUGGACGGCGACAACAGCCACGUGGAAAUGAAGCUGGCCGUGGACGAGGAAGAGAACGCCGACAACAACACCAAGGCCAACGUGACCAAGCCUAAGAGAUGCAGCGGCAGCAUCUGCUACGGCACAAUCGCCGUGAUCGUGUUCUUCCUGAUCGGCUUUAUGAUCGGCUACCUGGGCUACUGCAAGAGCAGUGAUGGACCUGGCGAAACAGGCGGAGGCGGAGGAUCUGGUGGCGGAGGAAGCGGUGGCGGCGGUUCUGCUCCUACAAGCAGCAGCACCAAGAAAACCCAGCUGCAACUGGAACAUCUGCUGCUGGACCUGCAGAUGAUCCUGAACGGCAUCAACAACUACAAGAACCCCAAGCUGACCCGGAUGCUGACCUUCAAGUUCUACAUGCCCAAGAAGGCCACCGAGCUGAAGCACCUCCAGUGCCUGGAAGAGGAACUGAAGCCCCUGGAAGAAGUGCUGAAUCUGGCCCAGAGCAAGAACUUCCACCUGAGGCCUAGGGACCUGAUCAGCAACAUCAAUGUGAUCGUGCUGGAACUCAAGGGCAGCGAGACAACCUUCAUGUGCGAGUACGCCGACGAGACAGCUACCAUCGUGGAAUUUCUGAACCGGUGGAUCACCUUCUGCCAGAGCAUCAUCAGCACCCUGACCUGAGCUGCCUUCUGCGGGGCUUGCCUUCUGGCCAUGCCCUUCUUCUCUCCCUUGCACCUGUACCUCUUGGUCUUUGAAUAAAGCCUGAGUAGGAAGUCUAGAAAAAAAAAAAAAAAAAAAAAAAAAAAAAAAAAAAAAAAAAAAAAAAAAAAAAAAAAAAAAAAAAAAAAAAAAAAAAAAA

SEQ ID NO: 22 - protein, 2060 aa, synthetic construct
  source 1..2060: note = membrane-bound IL-12 mRNA
GGAAAUAAGAGAGAAAAGAAGAGUAAGAAGAAAUAUAAGAGCCACCAUGAUGGUGGACGGCGACAACAGCCACGUGGAAAUGAAGCUGGCCGUGGACGAGGAAGAGAACGCCGACAACAACACCAAGGCCAACGUGACCAAGCCUAAGAGAUGCAGCGGCAGCAUCUGCUACGGCACAAUCGCCGUGAUCGUGUUCUUCCUGAUCGGCUUUAUGAUCGGCUACCUGGGCUACUGCAAGAGCAGUGAUGGACCUGGCGAAACAGGCGGAGGCGGAGGAUCUGGUGGCGGAGGAAGUGGCGGCGGAGGUUCUAUUUGGGAGCUGAAGAAAGACGUGUACGUGGUGGAACUGGACUGGUAUCCCGAUGCUCCUGGCGAGAUGGUGGUGCUGACCUGCGAUACCCCUGAAGAGGACGGCAUCACCUGGACACUGGAUCAGUCUAGCGAGGUGCUCGGCAGCGGCAAGACCCUGACCAUCCAAGUGAAAGAGUUUGGCGACGCCGGCCAGUACACCUGUCACAAAGGCGGAGAAGUGCUGAGCCACAGCCUGCUGCUGCUCCACAAGAAAGAGGAUGGCAUUUGGAGCACCGACAUCCUGAAGGACCAGAAAGAGCCCAAGAACAAGACCUUCCUGAGAUGCGAGGCCAAGAACUACAGCGGCCGGUUCACAUGUUGGUGGCUGACCACCAUCAGCACCGACCUGACCUUCAGCGUGAAGUCCAGCAGAGGCAGCAGUGAUCCUCAGGGCGUUACAUGUGGCGCCGCUACACUGUCUGCCGAAAGAGUGCGGGGCGAUAACAAAGAAUACGAGUACAGCGUGGAAUGCCAAGAGGACAGCGCCUGUCCAGCCGCCGAAGAGUCUCUGCCUAUCGAAGUGAUGGUCGACGCCGUGCACAAGCUGAAGUACGAGAACUACACCAGCAGCUUUUUCAUCCGGGACAUCAUCAAGCCCGAUCCUCCAAAGAACCUGCAGCUGAAGCCUCUGAAGAACAGCAGACAGGUGGAAGUGUCCUGGGAGUACCCCGACACCUGGUCUACACCCCACAGCUACUUCAGCCUGACCUUUUGCGUGCAAGUGCAGGGCAAGUCCAAGCGCGAGAAAAAGGACCGGGUGUUCACCGACAAGACCAGCGCCACCGUGAUCUGCAGAAAGAACGCCAGCAUCAGCGUCAGAGCCCAGGACCGGUACUACAGCAGCUCUUGGAGCGAAUGGGCCAGCGUGCCAUGUAGCGGAGGUGGUGGUAGCGGAGGCGGCGGAAGCGGCGGUGGUGGAUCAGGUGGUGGUGGCUCUAGAAACCUGCCAGUGGCUACCCCUGAUCCUGGCAUGUUCCCUUGUCUGCACCACAGCCAGAACCUGCUGAGAGCCGUGUCCAACAUGCUGCAGAAGGCCAGACAGACCCUGGAAUUCUACCCCUGCACCAGCGAGGAAAUCGACCACGAGGACAUCACCAAGGAUAAGACCAGCACCGUGGAAGCCUGCCUGCCUCUGGAACUGACCAAGAACGAGAGCUGCCUGAACAGCCGGGAAACCUCCUUCAUCACCAACGGCUCUUGCCUGGCCAGCAGAAAGACAAGCUUCAUGAUGGCCCUGUGCCUGAGCAGCAUCUACGAGGACCUGAAGAUGUACCAGGUGGAAUUCAAGACCAUGAACGCCAAGCUGCUGAUGGACCCCAAGCGGCAGAUCUUCCUGGACCAGAACAUGCUGGCUGUGAUCGACGAGCUGAUGCAGGCCCUGAACUUCAACAGCGAGACAGUGCCCCAGAAGUCUAGCCUGGAAGAACCCGACUUCUACAAGACCAAGAUCAAGCUGUGCAUCCUGCUGCACGCCUUCCGGAUCAGAGCCGUGACCAUCGACAGAGUGAUGAGCUACCUGAACGCCUCCUGAGCUGCCUUCUGCGGGGCUUGCCUUCUGGCCAUGCCCUUCUUCUCUCCCUUGCACCUGUACCUCUUGGUCUUUGAAUAAAGCCUGAGUAGGAAGUCUAGAAAAAAAAAAAAAAAAAAAAAAAAAAAAAAAAAAAAAAAAAAAAAAAAAAAAAAAAAAAAAAAAAAAAAAAAAAAAAAAA

SEQ ID NO: 23 - protein, 24 aa, synthetic construct
  source 1..24: note = CpG ODN 2006
TCGTCGTTTTGTCGTTTTGTCGTT

(19) United States
(12) Patent Application Publication
(10) Pub. No.: US 2025/0339530 A1
(43) Pub. Date: Nov. 6, 2025

(54) ENHANCED ANTIGEN PRESENTING CELL FORMULATIONS

(71) Applicant: STEMCELL TECHNOLOGIES CANADA INC., Vancouver (CA)

(72) Inventors: Howard Bernstein, Watertown, MA (US); Scott McNabb Loughhead, Watertown, MA (US); Emrah Ilker Ozay, Watertown, MA (US); Michael Finnan Maloney, Watertown, MA (US)

(21) Appl. No.: 18/997,650

(22) PCT Filed: Jul. 28, 2023
(86) PCT No.: PCT/US2023/071264

§ 371 Date: Jan. 22, 2025

Related U.S. Application Data
(60) Provisional application No. 63/369,715, filed on Jul. 28, 2022.

(51) Int. Cl. A61K 40/24; A61K 39/00; A61K 39/12; A61K 40/30; A61K 40/34; A61K 40/35; A61K 40/46; A61K 47/42; C12N 5/00

(52) U.S. Cl. CPC A61K 40/24; A61K 39/12; A61K 40/30; A61K 40/34; A61K 40/35; A61K 40/46; A61K 47/42; C12N 5/525; A61K 2039/5154; A61K 2039/5156; A61K 2039/575; C12N 2710/20034

(57) ABSTRACT

The present application provides formulations of enhanced antigen presenting cells (“enhanced APCs”), wherein the formulation comprises: the enhanced APCs comprising an antigen (e.g., a human papillomavirus (HPV) antigen) and one or more of the following: cryopreservation medium, hypothermic preservation medium, and human serum albumin. Also provided herein are methods of producing such formulations and the enhanced APCs.

### CROSS-REFERENCE TO RELATED APPLICATION

This PCT application claims the priority benefit of U.S. Provisional Application No. 63/369,715, filed Jul. 28, 2022, which is herein incorporated by reference in its entirety.

### REFERENCE TO SEQUENCE LISTING SUBMITTED ELECTRONICALLY

The content of the sequence listing is submitted electronically (Name: 4821_087PC01_SequenceListing_ST26.XML; Size: 36,447 bytes; and Date of Creation: Jul. 28, 2023) and is filed with the application herein incorporated by reference in its entirety.

### FIELD OF THE DISCLOSURE

The present disclosure relates generally to formulations comprising enhanced antigen presenting cells (e.g., comprising one or more HPV antigens and exhibiting increased expression of a co-stimulatory molecule and/or a cytokine) that are capable of activating T cells in an HLA-agnostic manner. Also provided are methods of manufacturing such formulations.

### BACKGROUND OF THE DISCLOSURE

Human papillomavirus, or HPV, is a virus that infects many people. In fact, more than 75% of women and men will be infected at some point in life. While most HPV infections are subclinical and will cause no physical symptoms, in some people infections can cause growths known as papillomas, and can even cause cancers of the cervix, vulva, vagina, penis, oropharynx and anus. In particular, HPV16 and HPV18 are known to cause around 70% of cervical cancer cases. There are currently no treatments available for HPV itself. Current treatment options generally aim to treat the various ailments that can be associated with HPV infection. While vaccine (e.g., GARDASIL®) has been approved, it is strictly preventive in nature and not without side effects. Therefore, new and alternative treatment options for HPV infection is needed.

All references cited herein, including patent applications and publications, are incorporated by reference in their entirety. The patent publications WO 2013/059343, WO 2015/023982, WO 2016/070136, WO2017041050, WO2017008063, WO 2017/192785, WO 2017/192786, WO 2019/178005, WO 2019/178006, WO 2020/072833, WO 2020/154696, and WO 2020/176789, US 20180142198, and US 20180201889 are hereby expressly incorporated by reference in their entirety.

### BRIEF SUMMARY OF THE DISCLOSURE

Provided herein is a pharmaceutical formulation comprising: (a) enhanced antigen presenting cells (“enhanced APCs”), (b) a cryopreservation medium, and (c) a solution comprising human serum albumin (“human serum albumin solution”), wherein the enhanced APCs are capable of activating T cells in an HLA agnostic manner. Also provided herein is a pharmaceutical formulation comprising: (a) enhanced antigen presenting cells (“enhanced APCs”), (b) a cryopreservation medium, (c) a hypothermic preservation medium, and (d) a solution comprising human serum albumin (“human serum albumin solution”), wherein the enhanced APCs are capable of activating T cells in an HLA agnostic manner.

In some aspects, the human serum albumin solution comprises 25% human serum albumin.

In some aspects, the formulation comprises about 5×10⁶ enhanced APCs to about 1×10⁹ enhanced APCs. In some aspects, the formulation comprises about 1×10⁴ enhanced APCs/mL to about 1×10⁹ enhanced APCs/mL. In some aspects, the formulation comprises about 1×10⁶ enhanced APCs/mL to about 1×10⁸ enhanced APCs/mL. In some aspects, the formulation comprises about 1.1×10⁷ enhanced APCs/mL.

In some aspects, the viability of the enhanced APCs is at least about 70%, at least about 80%, at least about 90%, or about 100%. In some aspects, the enhanced APCs in the formulation maintain about ≥70% viability following storage for at least about 12 months at ≤−140° C.

In some aspects, the formulation comprises cryopreservation medium, which is at a concentration of about 40% to about 95% (w/w). In some aspects, the cryopreservation medium is at a concentration of about 50% (w/w). In some aspects, the formulation comprises a hypothermic preservation medium, which is at a concentration of about 25% to about 35% (w/w). In some aspects, the hypothermic preservation medium is at a concentration of about 30% (w/w). In some aspects, the formulation comprises a human serum albumin solution, which is at a concentration of about 15% to about 25% (w/w). In some aspects, the human serum albumin solution is at a concentration of about 20% (w/w). In some aspects, the formulation has a pH which is about 6.0 to about 8.5. In some aspects, the pH of the formulation is between about 7.0 to about 7.9.

Provided herein is a pharmaceutical formulation comprising: (a) enhanced antigen present cells (“enhanced APCs”) at an amount of about 5×10⁶ enhanced APCs to about 1×10⁹ enhanced APCs; (b) a cryopreservation medium at a concentration of about 40% (w/w) to about 95% (w/w); (c) a hypothermic preservation medium at a concentration of about 25% (w/w) to about 35% (w/w); (d) a solution comprising about 25% human serum albumin (“human serum albumin solution”) at a concentration of about 15% (w/w) to about 25% (w/w); wherein the pH of the formulation is about 6.0 to about 8.5, and wherein the enhanced APCs are capable of activating T cells in an HLA agnostic manner.

Provided herein is a pharmaceutical formulation comprising: (a) enhanced antigen present cells (“enhanced APCs”) at an amount of about 1.05×10⁸ enhanced APCs; (b) a cryopreservation medium at a concentration of about 50% (w/w); (c) a hypothermic preservation medium at a concentration of about 30% (w/w); (d) a solution comprising about 25% human serum albumin (“human serum albumin solution”) at a concentration of about 20% (w/w); wherein the pH of the formulation is about 7.0 to about 7.9, and wherein the enhanced APCs are capable of activating T cells in an HLA agnostic manner.

Provided herein is a pharmaceutical formulation comprising: (a) enhanced antigen present cells (“enhanced APCs”) at an amount of about 7.6×10⁶ enhanced APCs; (b) a cryopreservation medium at a concentration of about 50% (w/w); (c) a hypothermic preservation medium at a concentration of about 30% (w/w); (d) a solution comprising about 25% human serum albumin (“human serum albumin solution”) at a concentration of about 20% (w/w); wherein the pH of the formulation is about 7.0 to about 7.9, and wherein the enhanced APCs are capable of activating T cells in an HLA agnostic manner.

Provided herein is a pharmaceutical formulation comprising: (a) enhanced antigen present cells (“enhanced APCs”) at a concentration of about 1×10⁴ enhanced APCs/mL to about 1×10⁹ enhanced APCs/mL; (b) a cryopreservation medium at a concentration of about 40% (w/w) to about 95% (w/w); (c) a hypothermic preservation medium at a concentration of about 25% (w/w) to about 35% (w/w); (d) a solution comprising about 25% human serum albumin (“human serum albumin solution”) at a concentration of about 15% (w/w) to about 25% (w/w); wherein the pH of the formulation is about 6.0 to about 8.5, and wherein the enhanced APCs are capable of activating T cells in an HLA agnostic manner.

Provided herein is a pharmaceutical formulation comprising: (a) enhanced antigen present cells (“enhanced APCs”) at a concentration of about 1.1×10⁷ enhanced APCs/mL; (b) a cryopreservation medium at a concentration of about 50% (w/w); (d) a hypothermic preservation medium at a concentration of about 30% (w/w); (c) a solution comprising about 25% human serum albumin (“human serum albumin solution”) at a concentration of about 20% (w/w); wherein the pH of the formulation is about 7.0 to about 7.9, and wherein the enhanced APCs are capable of activating T cells in an HLA agnostic manner.

Provided herein is a pharmaceutical formulation comprising: (a) enhanced antigen present cells (“enhanced APCs”) at a concentration of about 8.5×10⁶ enhanced APCs/mL; (b) a cryopreservation medium at a concentration of about 50% (w/w); (d) a hypothermic preservation medium at a concentration of about 30% (w/w); (c) a solution comprising about 25% human serum albumin (“human serum albumin solution”) at a concentration of about 20% (w/w); wherein the pH of the formulation is about 7.0 to about 7.9, and wherein the enhanced APCs are capable of activating T cells in an HLA agnostic manner.

Provided herein is a pharmaceutical formulation comprising: (a) about 1.05×10⁸ enhanced APCs, (b) about 4.99 g of a cryopreservation medium, (c) about 2.00 g of a solution comprising 25% human serum albumin (“human serum albumin solution”); wherein the pH of the formulation is about 7.0 to about 7.9; and wherein the enhanced APCs are capable of activating T cells in an agnostic manner.

Provided herein is a pharmaceutical formulation comprising: (a) about 1.05×10⁸ enhanced APCs, (b) about 4.99 g of a cryopreservation medium, (c) about 2.99 g of a hypothermic preservation medium, (d) about 2.00 g of a solution comprising 25% human serum albumin (“human serum albumin solution”); wherein the pH of the formulation is about 7.0 to about 7.9; and wherein the enhanced APCs are capable of activating T cells in an agnostic manner.

For any of the formulations provided herein comprising a cryopreservation medium, in some aspects, the cryopreservation medium is CryoStor® CS10. For any of the formulations provided herein comprising a hypothermic preservation medium, in some aspects, the hypothermic preservation medium is HypoThermasol® FRS.

In some aspects, formulations provided herein are sterile. In some aspects, the formulation comprises less than about 5 EU/mL of endotoxin. In some aspects, the formulation comprises less than about 4.2 EU/mL endotoxin. In some aspects, the formulation is free of mycoplasma.

Pharmaceutical formulations provided herein comprise enhanced APCs, wherein the enhanced APCs comprise T cells, B cells, NK cells, monocytes, or combinations thereof. In some aspects, the enhanced APCs comprise an antigen, wherein the antigen comprises a human papillomavirus (HPV) antigen. In some aspects, the HPV comprises HPV-16 or HPV-18. In some aspects, the antigen comprises a peptide derived from HPV E6 and/or HPV E7. In some aspects, the antigen comprises a peptide derived from HPV E6 and a peptide from HPV E7. In some aspects, the antigen comprises the amino acid sequence set forth in any one of SEQ ID NOs: 14-17. In some aspects, the antigen comprises a first antigen comprising the amino acid sequence set forth in SEQ ID NO: 14 and a second antigen comprising the amino acid sequence set forth in SEQ ID NO: 16.

In some aspects, a formulation described herein comprises enhanced APCs, wherein the enhanced APCs exhibit increased expression of a co-stimulatory molecule as compared to corresponding non-enhanced APCs (“reference APCs”). In some aspects, the co-stimulatory molecule comprises CD86. In some aspects, a formulation described herein comprise enhanced APCs, wherein the enhanced APCs exhibit increased expression of a cytokine as compared to corresponding non-enhanced APCs (“reference APCs”). In some aspects, the cytokine comprises a membrane-bound cytokine. In some aspects, the cytokine comprises IL-2, IL-12, or both.

In some aspects, a formulation provided herein comprises enhanced APCs, wherein the enhanced APCs have been prepared by passing a cell suspension comprising input APCs through a cell-deforming constriction, thereby causing perturbations in the APCs such that a nucleic acid encoding the antigen, a nucleic acid encoding the co-stimulatory molecule and/or a nucleic acid encoding the cytokine enters the APCs through the perturbations when contacted with the APCs, and thereby producing the enhanced APCs. In some aspects, the cell suspension comprising the input APCs have been cultured in combination with the nucleic acid encoding the antigen, the nucleic acid encoding the co-stimulatory molecule, and/or the nucleic acid encoding the cytokine, such that the nucleic acid encoding the antigen, the nucleic acid encoding the co-stimulatory molecule, and/or the nucleic acid encoding the cytokine are in contact with the input APCs.

In some aspects, the nucleic acid encoding the antigen, the nucleic acid encoding the co-stimulatory molecule, and/or the nucleic acid encoding the cytokine is a mRNA. In some aspects, the cell-deforming constriction comprises a diameter, which is about 4.2 μm to about 6 μm or about 4.2 μm to about 4.8 μm.

In some aspects, the enhanced APCs have been conditioned in a medium comprising an adjuvant. In some aspects, the enhanced APCs have been conditioned in a medium comprising an adjuvant for about 2 hours to about 10 hours. In some aspects, the enhanced APCs have been conditioned in a medium comprising an adjuvant for about 3 hours to about 6 hours. In some aspects, the enhanced APCs have been conditioned in a medium comprising an adjuvant for about 4 hours. In some aspects, the enhanced APCs have been conditioned in a medium comprising an adjuvant at about 37° C. In some aspects, the adjuvant comprises a CpG oligodeoxynucleotide (ODN), LPS, IFN-α, STING agonists, RIG-I agonists, poly I. C, R837, R848, a TLR3 agonist, a TLR4 agonist, or a TLR 9 agonist. In some aspects, the adjuvant is ODN.

Some aspects of the present disclosure relates to vials comprising any of the formulations provided herein.

Also provided herein is a method of producing a formulation comprising enhanced antigen presenting cells (“enhanced APCs”) which are capable of activating T cells in an HLA agnostic manner, the method comprising combining the enhanced APCs, a cryopreservation medium, and a human serum albumin solution. For such a method, in some aspects, the human serum albumin solution comprises 25% human serum albumin. In some aspects, after the combining, the formulation comprises: (a) about 5×10⁶ enhanced APCs to about 1×10⁹ enhanced APCs; (b) the cryopreservation medium at a concentration of about 40% (w/w) to about 95% (w/w); and (c) the human serum albumin solution at a concentration of about 15% (w/w) to about 25% (w/w); and wherein the formulation has a pH of about 6.0 to about 8.5. In some aspects, after the combining, the formulation comprises: (a) the enhanced APCs at a concentration of about 1.1×10⁷ enhanced APCs/mL; (b) the cryopreservation medium at a concentration of about 50% (w/w); and (c) the human serum albumin solution at a concentration of about 20% (w/w); and wherein the formulation has a pH of about 7.0 to about 7.9. In some aspects, after the combining, the formulation comprises: (a) about 1.05×10⁸ enhanced APCs, (b) about 4.99 g of the cryopreservation medium, and (c) about 2.00 g of the human serum albumin solution; and wherein the pH of the formulation is about 7.0 to about 7.9.

Provided herein is a method of producing a formulation comprising enhanced antigen presenting cells (“enhanced APCs”) which are capable of activating T cells in an HLA agnostic manner, the method comprising combining the enhanced APCs, a cryopreservation medium, a hypothermic preservation medium, and a human serum albumin. For such a method, in some aspects, the human serum albumin solution comprises 25% human serum albumin. In some aspects, after the combining, the formulation comprises: (a) about 5×10⁶ enhanced APCs to about 1×10⁹ enhanced APCs; (b) the cryopreservation medium at a concentration of about 40% (w/w) to about 95% (w/w); (c) the hypothermic preservation medium at a concentration of about 25% (w/w) to about 35% (w/w); and (d) the human serum albumin solution at a concentration of about 15% (w/w) to about 25% (w/w); and wherein the pH of the formulation is about 6.0 to about 8.5. In some aspects, after the combining, the formulation comprises: (a) about 1.1×10⁷ enhanced APCs/mL; (b) the cryopreservation medium at a concentration of about 50% (w/w); and (c) the human serum albumin solution at a concentration of about 20% (w/w); and wherein the formulation has a pH of about 7.0 to about 7.9. In some aspects, after the combining, the formulation comprises: (a) about 1.05×10⁸ enhanced APCs, (b) about 4.99 g of the cryopreservation medium, and (c) about 2.00 g of the human serum albumin solution; and wherein the pH of the formulation is about 7.0 to about 7.9.

For the above methods of producing a formulation, in some aspects, the cryopreservation medium comprises CryoStor® CS10. In some aspects, the hypothermic preservation medium comprises HypoThermasol® FRS.

For any of the producing methods provided above, in some aspects, the method comprises passing a cell suspension which comprises input APCs through a cell-deforming constriction, thereby causing perturbations in the APCs such that a nucleic acid encoding an antigen, a nucleic acid encoding a co-stimulatory molecule, and/or a nucleic acid encoding a cytokine enters the input APCs through the perturbations when contacted with the APCs, and thereby producing the enhanced APCs. In some aspects, culturing the cell suspension with the nucleic acid encoding an antigen, the nucleic acid encoding a co-stimulatory molecule, and/or the nucleic acid encoding a cytokine, such that the nucleic acid encoding an antigen, the nucleic acid encoding a co-stimulatory molecule, and/or the nucleic acid encoding a cytokine are in contact with the input APCs. In some aspects, the nucleic acid encoding an antigen, the nucleic acid encoding a co-stimulatory molecule, and/or the nucleic acid encoding a cytokine is a mRNA.

For any of the producing methods provided above, in some aspects, the antigen comprises a human papillomavirus (HPV) antigen. In some aspects, the HPV comprises is HPV-16 or HPV-18. In some aspects, the antigen comprises a peptide derived from HPV E6 and/or HPV E7. In some aspects, the antigen comprises a peptide derived from HPV E6 and a peptide from HPV E7. In some aspects, the antigen comprises the amino acid sequence set forth in any one of SEQ ID NOs: 14-17. In some aspects, the antigen comprises a first antigen comprising the amino acid sequence set forth in SEQ ID NO: 14 and a second antigen comprising the amino acid sequence set forth in SEQ ID NO: 16. In some aspects, the co-stimulatory molecule comprises CD86. In some aspects, the cytokine comprises a membrane-bound cytokine. In some aspects, the cytokine comprises IL-2, IL-12, or both.

### BRIEF DESCRIPTION OF THE FIGURES

FIG. 1 provides a schematic of an exemplary enhanced APC that can be included in the pharmaceutical formulations provided herein. As shown, five different mRNA molecules: (1) encoding the HPV-16 E6 protein comprising multi-epitopes (e.g., full-length protein), (2) encoding the HPV-16 E7 protein comprising multi-epitopes (e.g., full-length protein), (3) encoding CD86 (co-stimulatory molecule), (4) encoding a membrane-bound IL-2 (cytokine), and (5) encoding a membrane-bound IL-12 (cytokine). Once the mRNAs are introduced into the cells (e.g., via squeeze processing), the mRNAs are translated and the encoded protein is expressed by the APC.

FIGS. 2A-2C show exemplary flow plots using in calculating the viability of the enhanced APCs included in the pharmaceutical formulations described herein. Percent viability of the cells was calculated with the following formula: Viability %=(Total AO⁺ cells—Dead DAPI⁺ cells)/Total AO⁺ Cells×100. “Total AO⁺ cells”=number of events captured in the Acridine Orange positive gate (FIGS. 2A and 2C, which show the AO intensity and diameter, respectively). “Dead DAPI⁺ cells”=number of events captured in the DAPI positive gate (FIG. 2B). the live cell count and viability percentage of nucleated cells (eAPCs). For the particular flow plots shown, the % viability of the enhanced APCs was about 92.1%.

FIG. 3 provides an exemplary flow plot showing that the enhanced APCs described herein express CD45+(a marker for nucleated hematopoietic cells, e.g., PBMCs, e.g., immune cells).

FIGS. 4A and 4B provide exemplary qPCR analysis showing the amount of delivered mRNA present in enhanced APCs described herein (FIG. 4A) and control cells (i.e., unprocessed PBMCs) (FIG. 4B). Specifically, the mRNAs shown include: mRNA encoding the HPV-16 E6 protein (“E6”), mRNA encoding the HPV-16 E7 protein (“E7”), mRNA encoding CD86 (“CD86), mRNA encoding membrane-bound IL-2 (“mbIL-2”), and mRNA encoding membrane-bound IL-12 (“mbIL-12”).

FIGS. 5A-5D shows the frequency of cellular subtypes that can be found within the enhanced APCs described herein. The cellular subtypes shown include: B cells (CD19*; FIG. 5A), monocytes (CD14+; FIG. 5B), T cells (CD3+) and NK cells (CD56*) (FIG. 5C for both cell types), and granulocytes (CD66+; FIG. 5D).

FIG. 6 shows the percentage of Annexin V positive cells observed within the enhanced APCs described herein, as measured using flow cytometry.

FIGS. 7A-7D provide comparison of the translation efficiency (as measured using flow cytometry) of the CD86 mRNA, membrane-bound IL-2 mRNA, and membrane-bound IL-12 mRNA in the different cellular subtypes present in the enhanced APCs described herein after squeeze processing. FIG. 7A shows the percentage of B cells within the enhanced APCs that expressed CD86, membrane-bound IL-2 (mbIL-2), and membrane-bound IL-12 (mbIL-12) after squeeze processing. FIG. 7B shows the percentage of T cells within the enhanced APCs that expressed CD86, mbIL-2, and mbIL-12 after squeeze processing. FIG. 7C shows the percentage of monocytes cells within the enhanced APCs that expressed CD86, mbIL-2, and mbIL-12 after squeeze processing. FIG. 7D shows the percentage of NK cells within the enhanced APCs that expressed CD86, mbIL-2, and mbIL-12 after squeeze processing.

FIGS. 8A and 8B show the translation of E6 mRNA and E7 mRNA, respectively, as demonstrated by Western Blotting in the enhanced APCs after squeeze processing.

### DETAILED DESCRIPTION OF THE DISCLOSURE

The present application generally relates to pharmaceutical formulations comprising a population of antigen-presenting cells which have been modified such that APCs exhibit one or more enhanced properties. Non-limiting examples of such enhanced properties are provided throughout the present disclosure. Unless indicated otherwise, the terms “enhanced APCs” (or derivatives thereof) and “modified APCs”) (or derivatives thereof) are used interchangeably to described such APCs. As further described herein, the enhanced APCs differ from other APCs in that the cells are capable of activating T cells in a HLA-agnostic manner. Accordingly, the pharmaceutical formulations described herein are useful in various clinical settings and allow for the treatment of diverse subjects irrespective of HLA haplotype. Additional aspects of the present disclosure are provided further below.

### General Techniques

The techniques and procedures described or referenced herein are generally well understood and commonly employed using conventional methodology by those skilled in the art, such as, for example, the widely utilized methodologies described in Molecular Cloning: A Laboratory Manual (Sambrook et al., 4^{th} ed., Cold Spring Harbor Laboratory Press, Cold Spring Harbor, N.Y., 2012); Current Protocols in Molecular Biology (F. M. Ausubel, et al. eds., 2003); the series Methods in Enzymology (Academic Press, Inc.); PCR 2: A Practical Approach (M. J. MacPherson, B. D. Hames and G. R. Taylor eds., 1995); Antibodies, A Laboratory Manual (Harlow and Lane, eds., 1988); Culture of Animal Cells: A Manual of Basic Technique and Specialized Applications (R. I. Freshney, 6^{th} ed., J. Wiley and Sons, 2010); Oligonucleotide Synthesis (M. J. Gait, ed., 1984); Methods in Molecular Biology, Humana Press; Cell Biology: A Laboratory Notebook (J. E. Cellis, ed., Academic Press, 1998); Introduction to Cell and Tissue Culture (J. P. Mather and P. E. Roberts, Plenum Press, 1998); Cell and Tissue Culture: Laboratory Procedures (A. Doyle, J. B. Griffiths, and D. G. Newell, eds., J. Wiley and Sons, 1993-8); Handbook of Experimental Immunology (D. M. Weir and C. C. Blackwell, eds., 1996); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Ausubel et al., eds., J. Wiley and Sons, 2002); Immunobiology (C. A. Janeway et al., 2004); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane, Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 2011)

### Definitions

For purposes of interpreting this specification, the following definitions will apply and whenever appropriate, terms used in the singular will also include the plural and vice versa. In the event that any definition set forth below conflicts with any document incorporated herein by reference, the definition set forth shall control.

As used herein, the singular form term “a,” “an,” and “the” entity refers to one or more of that entity unless indicated otherwise. As such, the terms “a” (or “an” or “the”), “one or more,” and “at least one” can be used interchangeably herein.

The terms “comprising,” “having,” “containing,” and “including,” and other similar forms, and grammatical equivalents thereof, as used herein, are intended to be equivalent in meaning and to be open ended in that an item or items following any one of these words is not meant to be an exhaustive listing of such item or items, or meant to be limited to only the listed item or items. For example, an article “comprising” components A, B, and C can consist of (i.e., contain only) components A, B, and C, or can contain not only components A, B, and C but also one or more other components. As such, it is intended and understood that “comprises” and similar forms thereof, and grammatical equivalents thereof, include disclosure of aspects of “consisting essentially of” or “consisting of.”

Where a range of values is provided, it is understood that each intervening value, to the tenth of the unit of the lower limit, unless the context clearly dictates otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure.

The term “about” as used herein refers to the usual error range for the respective value readily known to the skilled person in this technical field. Reference to “about” a value or parameter herein includes (and describes) aspects that are directed to that value or parameter per se. For example, description referring to “about X” includes description of “X”.

As used herein, the term “HLA-agnostic manner” means independent of human leukocyte antigen (HLA) haplotype. Generally, T cell activation requires the recognition of antigens (as peptide fragments) presented on “Human leukocyte antigen” or “HLA,” which are expressed on certain cells. Because of the variability that exists in HLA molecules, certain peptides are restricted or binds only to certain HLA molecules. Therefore, whether a particular antigenic peptide fragment induces an immune response in a subject closely depends on the particular HLA molecules that are present within the subject. As is apparent from the present disclosure, because the enhanced APCs described herein are capable of activating T cells in a HLA-agnostic manner, they can have therapeutic effects in a much larger population.

As used herein, a “peripheral blood mononuclear cells” or “PBMCs” refers to a heterogeneous population of blood cells having a round nucleus. Examples of cells that can be found in a population of PBMCs include lymphocytes such as T cells, B cells, NK cells (including natural killer T cells (NKT cells) and cytokine-induced killer cells (CTK cells)) and monocytes such as macrophages and dendritic cells. PBMCs can be isolated by means known in the art. For example, PBMCs can be derived from peripheral blood of an individual based on density of PBMCs compared to other blood cells. In some aspects, PBMCs are derived from peripheral blood of an individual using Ficoll (e.g., a ficoll gradient). In some aspects, PBMCs are derived from peripheral blood of an individual using ELUTRA® cell separation system. PBMCs can be obtained from an individual undergoing apheresis.

As described and demonstrated herein, the enhanced APCs of the present disclosure are derived from PBMCs, e.g., by squeeze-processing PBMCs with one or more nucleic acid constructs (e.g., mRNA encoding an antigen, mRNA encoding a co-stimulatory molecule, and/or mRNA encoding a cytokine). The intracellular delivery of these constructs alters one or more properties of the PBMCs (e.g., expresses the encoded antigen on their surface such that they are capable of activating antigen-specific T cells and exhibits increased expression of a co-stimulatory molecule and/or cytokine), such that after the delivery, the enhanced cells are structurally and/or functionally different from the PBMCs.

As used herein “payload” refers to the material that is being delivered into, such as loaded in, the PBMCs. “Payload,” “cargo,” “delivery material,” and “compound” are used interchangeably herein as they refer to material that is being delivered into a cell. In some aspects, a payload can refer to a protein, a small molecule, a nucleic acid (e.g., RNA and/or DNA), a lipid, a carbohydrate, a macromolecule, a vitamin, a polymer, fluorescent dyes and fluorophores, carbon nanotubes, quantum dots, nanoparticles, and steroids. In some aspects, the payload can refer to a protein or small molecule drug. In some aspects, the payload can comprise one or more compounds. In some aspects, a payload comprises a nucleic acid molecule, e.g., encoding a HPV antigen, co-stimulatory molecule, and/or cytokine.

As used herein, “treatment” or “treating” is an approach for obtaining beneficial or desired results, including clinical results. For purposes of this disclosure, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delay or slowing the progression of the disease, ameliorating the disease state, providing a remission (partial or total) of the disease, decreasing the dose of one or more other medications required to treat the disease, delaying the progression of the disease, increasing or improving the quality of life, increasing weight gain, and/or prolonging survival. Also encompassed by “treatment” is a reduction of pathological consequence of cancer (such as, for example, tumor volume). The methods of the disclosure contemplate any one or more of these aspects of treatment.

As used herein, the term “enhance” can refer to the act of improving, boosting, heightening, or otherwise increasing the presence, or an activity of, a particular target. For example, enhancing an immune response can refer to any act leading to improving, boosting, heightening, or otherwise increasing an immune response. In one exemplary example, enhancing an immune response can refer to employing an antigen and/or adjuvant to improve, boost, heighten, or otherwise increase an immune response. In other examples, enhancing the expression of a nucleic acid can include, but not limited to increase in the transcription of a nucleic acid, increase in mRNA abundance (e.g., increasing mRNA transcription), decrease in degradation of mRNA, increase in mRNA translation, and so forth. In other examples, enhancing the expression of a protein can include, but not be limited to, increase in the transcription of a nucleic acid encoding the protein, increase in the stability of mRNA encoding the protein, increase in translation of the protein, increase in the stability of the protein, and so forth.

As used herein, the term “induce” can refer to the act of initiating, prompting, stimulating, establishing, or otherwise producing a result. For example, inducing an immune response can refer to any act leading to initiating, prompting, stimulating, establishing, or otherwise producing a desired immune response. In other examples, inducing the expression of a nucleic acid can include, but not limited to initiation of the transcription of a nucleic acid, initiation of mRNA translation, and so forth. In other examples, inducing the expression of a protein can include, but not be limited to, increase in the transcription of a nucleic acid encoding the protein, increase in the stability of mRNA encoding the protein, increase in translation of the protein, increase in the stability of the protein, and so forth.

The term “polynucleotide” or “nucleic acid” as used herein refers to a polymeric form of nucleotides of any length, either ribonucleotides or deoxyribonucleotides. Thus, this term includes, but is not limited to, single-, double- or multi-stranded DNA or RNA, genomic DNA, cDNA, DNA-RNA hybrids, or a polymer comprising purine and pyrimidine bases, or other natural, chemically or biochemically modified, non-natural, or derivatized nucleotide bases. The backbone of the polynucleotide can comprise sugars and phosphate groups (as can typically be found in RNA or DNA), or modified or substituted sugar or phosphate groups. Alternatively, the backbone of the polynucleotide can comprise a polymer of synthetic subunits such as phosphoramidates and thus can be an oligodeoxynucleoside phosphoramidate (P—NH2) or a mixed phosphoramidate-phosphodiester oligomer. In addition, a double-stranded polynucleotide can be obtained from the single stranded polynucleotide product of chemical synthesis either by synthesizing the complementary strand and annealing the strands under appropriate conditions, or by synthesizing the complementary strand de novo using a DNA polymerase with an appropriate primer. As described herein, a nucleic acid that can be delivered to a cell using the squeeze processing methods provided herein comprises a RNA (e.g., mRNA). As used herein, “RNA” comprises both self-amplifying RNA (e.g., self-amplifying mRNA) and non-self-amplifying RNA (e.g., non-self-amplifying mRNA). As used herein, the term “self-amplifying RNA” refers to a RNA molecule that can replicate in a host, resulting in an increase in the amount of RNA and proteins encoded by the RNA (e.g., antigens). As used herein, the term “mRNA” refers to any polynucleotides (either self-amplifying or non-self-amplifying) which encodes at least one polypeptide.

The terms “polypeptide” and “protein” are used interchangeably to refer to a polymer of amino acid residues, and are not limited to a minimum length. Such polymers of amino acid residues can contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present disclosure, a “polypeptide” refers to a protein which includes modifications, such as deletions, additions, and substitutions (generally conservative in nature), to the native sequence, as long as the protein maintains the desired activity. These modifications can be deliberate, as through site-directed mutagenesis, or can be accidental, such as through mutations of hosts which produce the proteins or errors due to PCR amplification.

As used herein, the term “adjuvant” refers to a substance which modulates and/or engenders an immune response. Generally, the adjuvant is administered in conjunction with an antigen to effect enhancement of an immune response to the antigen as compared to antigen alone. Various adjuvants are described herein.

The terms “CpG oligodeoxynucleotide” and “CpG ODN” herein refer to DNA molecules of 10 to 30 nucleotides in length containing a dinucleotide of cytosine and guanine separated by a phosphate (also referred to herein as a “CpG” dinucleotide, or “CpG”). The CpG ODNs of the present disclosure contain at least one unmethylated CpG dinucleotide. That is, the cytosine in the CpG dinucleotide is not methylated (i.e., is not 5-methylcytosine). CpG ODNs can have a partial or complete phosphorothioate (PS) backbone.

As used herein, by “pharmaceutically acceptable” or “pharmacologically compatible” is meant a material that is not biologically or otherwise undesirable, e.g., the material can be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug Administration.

For any of the structural and functional characteristics described herein, methods of determining these characteristics are known in the art.

As used herein, “microfluidic systems” refers to systems in which low volumes (e.g., mL, nL, pL, fL) of fluids are processed to achieve the discrete treatment of small volumes of liquids. Certain implementations described herein include multiplexing, automation, and high throughput screening. The fluids (e.g., a buffer, a solution, a payload-containing solution, or a cell suspension) can be moved, mixed, separated, or otherwise processed. In certain aspects described herein, microfluidic systems are used to apply mechanical constriction to a cell suspended in a buffer, inducing perturbations in the cell (e.g., holes) that allow a payload or compound to enter the cytosol of the cell.

The term “constriction” as used herein refers to a narrowed passageway. In some aspects, the constriction is a microfluidic channel, such as that contained within a microfluidic device. In some aspects, the constriction is a pore or contained within a pore. Where the constriction is a pore, in some aspects, the pore is contained in a surface. Unless indicated otherwise, the term constriction refers to both microfluidic channels and pores, as well as other suitable constrictions available in the art. Therefore, where applicable, disclosures relating to microfluidic channels can also apply to pores and/or other suitable constrictions available in the art. Similarly, where applicable, disclosures relating to pores can equally apply to microfluidic channels and/or other suitable constrictions available in the art.

The term “pore” as used herein refers to an opening, including without limitation, a hole, tear, cavity, aperture, break, gap, or perforation within a material. In some aspects, (where indicated) the term refers to a pore within a surface of a microfluidic device, such as those described in the present disclosure. In some aspects, (where indicated) a pore can refer to a pore in a cell wall and/or cell membrane.

The term “membrane” as used herein refers to a selective barrier or sheet containing pores. The term includes, but is not limited to, a pliable sheet-like structure that acts as a boundary or lining. In some aspects, the term refers to a surface or filter containing pores. This term is distinct from the term “cell membrane,” which refers to a semipermeable membrane surrounding the cytoplasm of cells.

The term “filter” as used herein refers to a porous article that allows selective passage through the pores. In some aspects, the term refers to a surface or membrane containing pores.

As used herein, the terms “deform” and “deformity” (including derivatives thereof) refer to a physical change in a cell. As described herein, as a cell passes through a constriction (such as those of the present disclosure), it experiences various forces due to the constraining physical environment, including but not limited to mechanical deforming forces and/or shear forces that causes perturbations in the cell membrane. As used herein, a “perturbation” within the cell membrane refers to any opening in the cell membrane that is not present under normal steady state conditions (e.g., no deformation force applied to the cells). Perturbation can comprise a hole, tear, cavity, aperture, pore, break, gap, perforation, or combinations thereof.

For any of the structural and functional characteristics described herein, methods of determining these characteristics are known in the art.

### Compositions of the Disclosure

### Enhanced APCs

Provided herein are pharmaceutical formulations comprising enhanced APCs which are capable of activating T cells in an HLA-agnostic manner.

In some aspects, the formulation comprises about 5×10³ to about 5×10¹⁰ enhanced APCs. In some aspects, the formulation comprises about 5×10⁴ to about 5×10⁹ enhanced APCs. In some aspects, the formulation comprises 5×10⁵ to about 5×10⁹ enhanced APCs. In some aspects, the formulation comprises 5×10⁶ to about 5×10⁹ enhanced APCs. In some aspects, the formulation comprises 5×10⁷ to about 5×10⁹ enhanced APCs. In some aspects, the formulation comprises 5×10⁸ to about 5×10⁹ enhanced APCs. In some aspects, the formulation comprises 5×10⁴ to about 5×10⁸ enhanced APCs. In some aspects, the formulation comprises 5×10⁴ to about 5×10⁷ enhanced APCs. In some aspects, the formulation comprises 5×10⁴ to about 5×10⁶ enhanced APCs. In some aspects, the formulation comprises 5×10⁴ to about 5×10⁵ enhanced APCs. In some aspects, the formulation comprises 5×10⁵ to about 5×10⁸ enhanced APCs. In some aspects, the formulation comprises 5×10⁶ to about 5×10⁸ enhanced APCs. In some aspects, the formulation comprises 5×10⁷ to about 5×10⁸ enhanced APCs. In some aspects, the formulation comprises 5×10⁵ to about 5×10⁷ enhanced APCs. In some aspects, the formulation comprises 5×10⁶ to about 5×10⁷ enhanced APCs.

In some aspects, the formulation comprises about 5×10⁴, about 1.0×10⁴, about 5×10⁵, about 1.0×10⁵, about 5×10⁶, about 1.0×10⁶, about 5×10⁷, about 0×10⁷, about 5×10⁸, about 1.0×10⁸, about 5×10⁹, about 1.0×10⁹, about 5.0×10⁹ enhanced APCs. In some aspects, the formulation comprises about 0.5×10⁴ to about 1.0×10⁴, about 1.0×10⁵ to about 0.5×10⁵, about 0.5×10⁵ to about 1.0×10⁵, about 1.0×10⁵ to about 0.5×10⁶, about 0.5×10⁶ to about 1.0×10⁶, about 1.0×10⁶ to about 0.5×10⁷, about 0.5×10⁷ to about 1.0×10⁷, about 1.0×10⁷ to about 0.5×10⁸, about 0.5×10⁸ to about 1.0×10⁸, about 1.0×10⁸ to about 0.5×10⁹, about 0.5×10⁹ to about 1.0×10⁹, or about 1.0×10⁹ to about 5×10⁹ enhanced APCs. In some aspects, the formulation comprises about 1×10⁷, about 2×10⁷, about 3×10⁷, about 4×10⁷, about 5×10⁷, about 6×10⁷, about 7×10⁷, about 8×10⁷, about 9×10⁷, about 1×10⁸, about 2×10⁸, about 3×10⁸, about 4×10⁸, about 5×10⁸, about 6×10⁸, about 7×10⁸, about 8×10⁸, about 9×10′, about 1×10⁹, about 2×10⁹, about 3×10⁹, about 4×10⁹, or about 5×10⁹ enhanced APCs.

In some aspects, the formulation comprises about 1×10⁶ to about 1×10⁹ enhanced APCs. In some aspects, the formulation comprises about 1×10⁷ to about 1×10⁹ enhanced APCs. In some aspects, the formulation comprises about 1×10⁸ to about 1×10⁹ enhanced APCs. In some aspects, the formulation comprises about 1×10⁶ enhanced APCs. In some aspects, the formulation comprises about 2×10⁶ enhanced APCs. In some aspects, the formulation comprises about 3×10⁶ enhanced APCs. In some aspects, the formulation comprises about 4×10⁶ enhanced APCs. In some aspects, the formulation comprises about 5×10⁶ enhanced APCs. In some aspects, the formulation comprises about 6×10⁶ enhanced APCs. In some aspects, the formulation comprises about 7×10⁶ enhanced APCs. In some aspects, the formulation comprises about 8×10⁶ enhanced APCs. In some aspects, the formulation comprises about 9×10⁶ enhanced APCs. In some aspects, the formulation comprises about 1×10⁷ enhanced APCs. In some aspects, the formulation comprises about 2×10⁷ enhanced APCs. In some aspects, the formulation comprises about 3×10⁷ enhanced APCs. In some aspects, the formulation comprises about 4×10⁷ enhanced APCs. In some aspects, the formulation comprises about 5×10⁷ enhanced APCs. In some aspects, the formulation comprises about 6×10⁷ enhanced APCs. In some aspects, the formulation comprises about 7×10⁷ enhanced APCs. In some aspects, the formulation comprises about 8×10⁷ enhanced APCs. In some aspects, the formulation comprises about 9×10⁷ enhanced APCs. In some aspects, the formulation comprises about 1×10⁸ enhanced APCs. In some aspects, the formulation comprises about 2×10⁸ enhanced APCs. In some aspects, the formulation comprises about 3×10⁸ enhanced APCs. In some aspects, the formulation comprises about 4×10⁸ enhanced APCs. the formulation comprises about 5×10⁸ enhanced APCs. the formulation comprises about 6×10⁸ enhanced APCs. the formulation comprises about 7×10⁸ enhanced APCs. the formulation comprises about 8×10⁸ enhanced APCs. the formulation comprises about 9×10⁸ enhanced APCs. the formulation comprises about 1×10⁹ enhanced APCs. In some aspects, the formulation comprises about 1.05×10⁸ enhanced APCs. In some aspects, the formulation comprises about 7.6×10⁶ enhanced APCs.

As further described herein, in some aspects, a formulation described herein is resuspended in a liquid medium. Accordingly, in some aspects, a formulation of the present disclosure comprises a liquid formulation. For such formulations, in some aspects, the enhanced cells are present in the formulation at a concentration of about 1×10⁴ to about 1×10⁹ enhanced APCs/mL. In some aspects, the enhanced cells are present at a concentration of about 1×10⁵ to about 1×10⁹ enhanced APCs/mL. In some aspects, the enhanced cells are present at a concentration of about 1×10⁶ to about 1×10⁹ enhanced APCs/mL. In some aspects, the enhanced cells are present at a concentration of about 1×10⁷ to about 1×10⁹ enhanced APCs/mL. In some aspects, the enhanced cells are present at a concentration of about 1×10⁸ to about 1×10⁹ enhanced APCs/mL. In some aspects, the enhanced cells are present at a concentration of about 1×10⁴ to about 1×10⁸ enhanced APCs/mL. In some aspects, the enhanced cells are present at a concentration of about 1×10⁵ to about 1×10⁸ enhanced APCs/mL. In some aspects, the enhanced cells are present at a concentration of about 1×10⁶ to about 1×10⁸ enhanced APCs/mL. In some aspects, the enhanced cells are present at a concentration of about 1×10⁷ to about 1×10⁸ enhanced APCs/mL. In some aspects, the enhanced cells are present at a concentration of about 1×10⁴ to about 1×10⁷ enhanced APCs/mL. In some aspects, the enhanced cells are present at a concentration of about 1×10⁵ to about 1×10⁷ enhanced APCs/mL. In some aspects, the enhanced cells are present at a concentration of about 1×10⁶ to about 1×10⁷ enhanced APCs/mL. In some aspects, the enhanced cells are present at a concentration of about 1×10⁴ to about 1×10⁶ enhanced APCs/mL. In some aspects, the enhanced cells are present at a concentration of about 1×10⁴ to about 1×10⁵ enhanced APCs/mL. In some aspects, the enhanced cells are present in the formulation at a concentration of about 1×10⁴ enhanced APCs/mL. In some aspects, the formulation comprises about any one of 1.0×10⁴, 0.5×10⁵, 1.0×10⁵, 0.5×10⁶, 1.0×10⁶, 0.5×10⁷, 1.0×10⁷, 0.5×10⁸, 1.0×10⁸, 0.5×10⁹, and 1.0×10⁹ enhanced APCs/mL. In some aspects, the formulation comprises any one of 0.5×10⁴ to about 1.0×10⁴, about 1.0×10⁴ to about 0.5×10⁵, about 0.5×10⁵ to about 1.0×10⁵, about 1.0×10⁵ to about 0.5×10⁶, about 0.5×10⁶ to about 1.0×10⁶, about 1.0×10⁶ to about 0.5×10⁷, about 0.5×10⁷ to about 1.0×10⁷, about 1.0×10⁷ to about 0.5×10⁸, about 0.5×10⁸ to about 1.0×10⁸, about 1.0×10⁸ to about 0.5×10⁹, or about 0.5×10⁹ to about 1.0×10⁹ enhanced APCs/mL. In some aspects, the formulation comprises about any one of 1×10⁶, 2×10⁶, 3×10⁶, 4×10⁶, 5×10⁶, 6×10⁶, 7×10⁶, 8×10⁶, 9×10⁶, and 1×107 enhanced APCs/mL. In some aspects, the enhanced APCs are present in the formulation at a concentration of about 1×10⁶ cells/mL, about 2×10⁶ cells/mL, about 3×10⁶ cells/mL, about 4×10⁶ cells/mL, about 5×10⁶ cells/mL, about 6×10⁶ cells/mL, about 7×10⁶ cells/mL, about 8×10⁶ cells/mL, about 9×10⁶ cells/mL, or about 1×10⁷ cells/mL. In some aspects, the enhanced APCs are present in the formulation at a concentration of about 1×10⁶ cells/mL. In some aspects, the enhanced APCs are present in the formulation at a concentration of about 2×10⁶ cells/mL. In some aspects, the enhanced APCs are present in the formulation at a concentration of about 3×10⁶ cells/mL. In some aspects, the enhanced APCs are present in the formulation at a concentration of about 4×10⁶ cells/mL. In some aspects, the enhanced APCs are present in the formulation at a concentration of about 5×10⁶ cells/mL. In some aspects, the enhanced APCs are present in the formulation at a concentration of about 6×10⁶ cells/mL. In some aspects, the enhanced APCs are present in the formulation at a concentration of about 7×10⁶ cells/mL. In some aspects, the enhanced APCs are present in the formulation at a concentration of about 8×10⁶ cells/mL. In some aspects, the enhanced APCs are present in the formulation at a concentration of about 9×10⁶ cells/mL. In some aspects, the enhanced APCs are present in the formulation at a concentration of about 1×10⁷ cells/mL. In some aspects, the enhanced APCs are present in the formulation at a concentration of about 1.1×10⁷ cells/mL. In some aspects, the enhanced APCs are present in the formulation at a concentration of about 8.5×10⁶ cells/mL.

The enhanced APCs described herein can comprise any suitable cells known in the art, as long as the cells can be enhanced to function in an HLA-agnostic manner (e.g., as described herein). Accordingly, in some aspects, a formulation described herein comprises enhanced APCs that are capable of activating T cells in an HLA-independent manner, wherein the enhanced APCs comprise T cells, B cells, NK cells, monocytes, or combinations thereof.

In some aspects, about 10% to about 90% of the enhanced APCs are T cells. In some aspects, about 25% to about 70% of the enhanced APCs are T cells. In some aspects, about 2% to about 20% of the enhanced APCs are B cells. In some aspects, about 2.5% to about 14% of the enhanced APCs are B cells. In some aspects, about 3.5% to about 35% of the enhanced APCs are NK cells. In some aspects, about 4% to about 25% of the enhanced APCs are NK cells.

In some aspects, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80% of the enhanced APCs are T cells. In some aspects, at least about 25% of the enhanced APCs are T cells. In some aspects, at least about 0.5%, at least about 1%, at least about 1.5%, at least about 2%, at least about 2.5%, at least about 3%, at least about 4%, at least about 5%, at least about 6%, at least about 7%, at least about 7.5%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 25%, or at least about 30% of the enhanced APCs are B cells. In some aspects, at least about 1.5% of the enhanced APCs are B cells. In some aspects, at least about 0.5%, at least about 1%, at least about 1.5%, at least about 2%, at least about 2.5%, at least about 3%, at least about 4%, at least about 5%, at least about 6%, at least about 7%, at least about 7.5%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 25%, or at least about 30% of the enhanced APCs are NK cells. In some aspects, at least about 3% of the enhanced APCs are NK cells. In some aspects, at least about at least about 1%, at least about 2%, at least about 3%, at least about 4%, at least about 5%, at least about 6%, at least about 7%, at least about 8%, at least about 9%, at least about 10%, at least about 12%, at least about 14%, at least about 16%, at least about 18%, at least about 20%, at least about 25%, at least about 30%, at least about 35% or at least about 40% of the enhanced APCs are monocytes. In some aspects, at least about 4% of the enhanced APCs are monocytes. In some aspects, at least about 25% of the enhanced APCs are T cells; at least about 1.5% of the enhanced APCs are B cells; at least about 3% of the enhanced APCs are NK cells; and at least about 4% of the enhanced APCs are monocytes.

In some aspects, not more than about 40%, not more than about 45%, not more than about 50%, not more than about 55%, not more than about 60%, not more than about 65%, not more than about 70%, not more than about 75%, not more than about 80%, not more than about 85%, or not more than about 90% of the enhanced APCs are T cells. In some aspects, not more than about 70% of the enhanced APCs are T cells. In some aspects, not more than about 5%, not more than about 10%, not more than about 12%, not more than about 14%, not more than about 16%, not more than about 18%, not more than about 20%, not more than about 22%, not more than about 25%, not more than about 30%, not more than about 35%, not more than about 40%, or not more than about 50% of the enhanced APCs are B cells. In some aspects, not more than about 30% of the enhanced APCs are B cells. In some aspects, not more than about not more than about 10%, not more than about 15%, not more than about 20%, not more than about 25%, not more than about 30%, not more than about 35%, not more than about 40%, not more than about 45%. not more than about 50% or not more than about 60% of the enhanced APCs are NK cells. In some aspects, not more than about 20% of the enhanced APCs are NK cells. In some aspects, not more than about 5%, not more than about 10%, not more than about 12%, not more than about 14%, not more than about 16%, not more than about 18%, not more than about 20%, not more than about 22%, not more than about 25%, not more than about 30%, not more than about 35%, not more than about 40%, or not more than about 50% of the enhanced APCs are monocytes. In some aspects, not more than about 45% of the enhanced APCs are monocytes. In some aspects, not more than about 80% of the enhanced APCs are T cells; not more than about 30% of the enhanced APCs are B cells; not more than about 20% of the enhanced APCs are NK cells; and not more than about 45% of the enhanced APCs are monocytes.

### Antigen

As is apparent from the present disclosure, the enhanced APCs described herein comprise an antigen. Accordingly, when a formulation described herein is administered to a subject, in some aspects, an immune response against the antigen is induced in the subject. In some aspects, where the antigen is associated with a particular disease or condition, the induced immune response can be useful in treating the particular disease or condition. Any suitable antigens can be used with the present disclosure.

In some aspects, an antigen comprises an antigen derived from a human papillomavirus (HPV) (also referred to herein as “HPV antigen). “HPV” refers more specifically to papillomavirus of human species origin and/or which are capable of infecting a human. More than 100 HPV genotypes have been identified at present time and they have been numbered following the chronological order of their isolation. By convention, the classification of HPV is based on the degree of relatedness of their genomes. A phylogenetic tree was constructed from the alignment of the available nucleotide sequences (Van Ranst et al., 1992, J. Gen. Virol. 73, 2653; De Villiers et al., 2004, Virology 324, 17-27). HPV can be divided into “high risk” (HR-HPV) and “low-risk” (LR-HPV). HR—HPV refers to HPV that are strongly associated with cellular transformation that can lead to lesions with potential to progress to malignant lesions. HR-HPV types include, without limitation, HPV-16, HPV-18, HPV-30, HPV-31, HPV-33, HPV-35, HPV-39, HPV-45, HPV-51, HPV-52, HPV-56, HPV-58, HPV-59, HPV-66, HPV-68, HPV-70 and HPV-85. LR-HPV refers to HPV that have a weak cellular transformation potential that can lead to benign lesions such as warts with low potential to progress to malignant lesions. LR-HPV types include, without limitation, HPV-6 and HPV-11. Unless indicated otherwise, the HPV antigen can be derived from any HPV.

In some aspects, the HPV antigen is derived from HPV-16, HPV-18, or both. In some aspects, the antigen is derived from HPV-16. In some aspects, the antigen is derived from HPV-18. Accordingly, in some aspects, a pharmaceutical formulation described herein comprises enhanced APCs which comprise a HPV antigen, wherein the enhanced APCs are capable of activating T cells in a HLA-agnostic manner. In some aspects, a pharmaceutical formulation comprises a HPV antigen derived from HPV-16, wherein the enhanced APCs are capable of activating T cells in a HLA-agnostic manner. In some aspects, a pharmaceutical formulation comprises a HPV antigen derived from HPV-18, wherein the enhanced APCs are capable of activating T cells in a HLA-agnostic manner.

In some aspects, the HPV antigen comprises a full-length HPV E6 protein (e.g., E6 protein of HPV-16 or HPV-18, which are both 158 amino acids in length). In some aspects, the HPV antigen comprises a full-length HPV E7 protein (e.g., E7 protein of HPV-16, which is 98 amino acids in length or E7 protein of HPV-18, which is 105 amino acids in length). The amino acid sequence for the full-length E6 protein of HPV-16 is set forth in SEQ ID NO: 14 (see Table 1 below). The amino acid sequence for the full-length E7 protein of HPV-16 is set forth in SEQ ID NO: 15 (see Table 1 below). The amino acid sequence for the full-length E6 protein of HPV-18 is set forth in SEQ ID NO: 16 (see Table 1 below). The amino acid sequence for the full-length E7 protein of HPV-18 is set forth in SEQ ID NO: 17 (see Table 1 below).

TABLE 1
Exemplary HPV E6 and E7 Protein Sequences

| | |
|---|---|
| HPV-16 E6 | MHQKRTAMFQDPQERPRKLPQLCTELQTTIHDIILECVYCKQQLLRREVY |
| UniProt: P03126 | DFAFRDLCIVYRDGNPYAVCDKCLKFYSKISEYRHYCYSLYGTTLEQQYN |
| (SEQ ID NO: 14) | KPLCDLLIRCINCQKPLCPEEKQRHLDKKQRFHNIRGRWTGRCMSCCRSS |
| | RTRRETQL |
| HPV-16 E7 | MHGDTPTLHEYMLDLQPETTDLYCYEQLNDSSEEEDEIDGPAGQAEPDRA |
| UniProt: P03129 | HYNIVTFCCKCDSTLRLCVQSTHVDIRTLEDLLMGTLGIVCPICSQKP |
| (SEQ ID NO: 15) | |
| HPV-18 E6 | MARFEDPTRRPYKLPDLCTELNTSLQDIEITCVYCKTVLELTEVFEFAFK |
| UniProt: P06463 | DLFVVYRDSIPHAACHKCIDFYSRIRELRHYSDSVYGDTLEKLINTGLYN |
| (SEQ ID NO: 16) | LLIRCLRCQKPLNPAEKLRHLNEKRRFHNIAGHYRGQCHSCCNRARQERL |
| | QRRRETQV |
| HPV-18 E7 | MHGPKATLQDIVLHLEPQNEIPVDLLCHEQLSDSEEENDEIDGVNHQHLP |
| UniProt: P06788 | ARRAEPQRHTMLCMCCKCEARIKLVVESSADDLRAFQQLFLNTLSFVCPW |
| (SEQ ID NO: 17) | CASQQ |

Accordingly, in some aspects, a formulation provided herein comprises enhanced APCs comprising a HPV antigen, wherein the HPV antigen comprises the amino acid sequence set forth in SEQ ID NO: 14, and wherein the enhanced APCs are capable of activating T cells in an HLA-agnostic manner. In some aspects, a formulation provided herein comprises enhanced APCs comprising a HPV antigen, wherein the HPV antigen comprises the amino acid sequence set forth in SEQ ID NO: 15, and wherein the enhanced APCs are capable of activating T cells in an HLA-agnostic manner. In some aspects, a formulation provided herein comprises enhanced APCs comprising a HPV antigen, wherein the HPV antigen comprises the amino acid sequence set forth in SEQ ID NO: 16, and wherein the enhanced APCs are capable of activating T cells in an HLA-agnostic manner. In some aspects, a formulation provided herein comprises enhanced APCs comprising a HPV antigen, wherein the HPV antigen comprises the amino acid sequence set forth in SEQ ID NO: 17, and wherein the enhanced APCs are capable of activating T cells in an HLA-agnostic manner.

In some aspects, the HPV antigen comprises a variant of the full-length HPV E6 protein (“HPV E6 variant”). In some aspects, the HPV E6 variant is a fragment of the full-length HPV E6 protein. For instance, in some aspects, the HPV E6 variant comprises about 5, about 10, about 20, about 30, about 40, about 50, about 60, about 70, about 80, about 90, about 100, about 110, about 120, about 130, about 140, about 150, or about 155-amino acid fragment of SEQ ID NO: 1 or SEQ ID NO: 16. In some aspects, the HPV E7 variant comprises about 5, about 10, about 20, about 30, about 40, about 50, about 60, about 70, about 80, about 90, or about 95-amino acid fragment of SEQ ID NO: 2. In some aspects, the HPV E7 variant comprises about 5, about 10, about 20, about 30, about 40, about 50, about 60, about 70, about 80, about 90, or about 100-amino acid fragment of SEQ ID NO: 17.

In some aspects, the HPV E6 variant comprises one or more amino acid substitutions as compared to the corresponding wild-type HPV E6 protein. Accordingly, in some aspects, the HPV E6 variant comprises one or more amino acid substitutions as compared to the sequence set forth in SEQ ID NO: 14. In some aspects, the HPV E6 variant comprises one or more amino acid substitutions as compared to the sequence set forth in SEQ ID NO: 16. In some aspects, the HPV E6 variant comprises an amino acid sequence that has at least about 75%, at least about 80%, at least about 85%, or about 99% sequence identity with the sequence set forth in SEQ ID NO: 14. In some aspects, the HPV E6 variant comprises an amino acid sequence that has at least about 75%, at least about 80%, at least about 85%, or about 99% sequence identity with the sequence set forth in SEQ ID NO: 16. Similarly, in some aspects, the HPV E7 variant comprises one or more amino acid substitutions as compared to the corresponding wild-type HPV E7 protein. In some aspects, the HPV E7 variant comprises one or more amino acid substitutions as compared to the sequence set forth in SEQ ID NO: 15. In some aspects, the HPV E7 variant comprises one or more amino acid substitutions as compared to the sequence set forth in SEQ ID NO: 17. In some aspects, the HPV E7 variant comprises an amino acid sequence that has at least about 75%, at least about 80%, at least about 85%, or about 99% sequence identity with the sequence set forth in SEQ ID NO: 15. In some aspects, the HPV E7 variant comprises an amino acid sequence that has at least about 75%, at least about 80%, at least about 85%, or about 99% sequence identity with the sequence set forth in SEQ ID NO: 17.

In some aspects, enhanced APCs of a formulation provided herein comprises multiple antigens. In some aspects, the enhanced APCs comprise a HPV antigen and a non-HPV antigen. For instance, in some aspects, the enhanced APCs comprise multiple HPV antigens. In some aspects, the enhanced APCs comprise at least two, at least three, at least four, or at least five HPV antigens. In some aspects, the enhanced APCs comprises two HPV antigens, wherein the first HPV antigen is the HPV E6 protein (e.g., full-length E6 protein of HPV-16) and the second HPV antigen is the HPV E7 protein (e.g., full-length E7 protein of HPV-16).

In some aspects, any of the antigens described herein can be introduced to an APC to produce the enhanced APCs using any suitable methods known in the art. Non-limiting examples of suitable methods for delivering one or more exogenous nucleotide sequences to a cell include: transfection (also known as transformation and transduction), electroporation, non-viral delivery, viral transduction, lipid nanoparticle delivery, and combinations thereof. In some aspects, an antigen (e.g., HPV antigen) is introduced to the APCs using a constriction-mediated delivery described herein. As further described elsewhere in the present disclosure, as the cells pass through the constriction, they become transiently deformed, such that cell membrane of the cells is perturbed. The perturbation within the cell membrane can allow various payloads (e.g., nucleic acids encoding a HPV antigen, co-stimulatory molecule, and/or a cytokine) to enter the cells through the perturbation (e.g., through diffusion). The specific process by which the cells pass through a constriction and become transiently deformed is referred to herein as “squeeze processing,” “squeeze delivery,” or “squeezing.”

Accordingly, in some aspects, the enhanced cells described herein (e.g., comprising a HPV antigen) have been passed through a constriction under a set of parameters, thereby causing a perturbation within the APCs such that the antigen is able to enter the APCs through the perturbation when contacted with the APCs. More specifically, in some aspects, enhanced cells of a formulation described herein have been passed through a constriction under a set of parameters, thereby causing a perturbation within the APCs such that a nucleic acid encoding a HPV E6 protein (e.g., full-length E6 protein of HPV-16) enters the APCs when contacted with the APCs. In some aspects, enhanced cells for a formulation described herein have been passed through a constriction under a set of parameters, thereby causing a perturbation within the APCs such that a nucleic acid encoding a HPV E7 protein (e.g., full-length E7 protein of HPV-16) enters the APCs when contacted with the APCs. In some aspects, enhanced cells for a formulation described herein have been passed through a constriction under a set of parameters, thereby causing a perturbation within the APCs such that a nucleic acid encoding a HPV E7 protein (e.g., full-length E7 protein of HPV-16) and a nucleic acid encoding a HPV E6 protein (e.g., full-length E6 protein of HPV-16) enters the APCs when contacted with the APCs. In some aspects, the nucleic acid encoding the HPV E7 protein and/or HPV E6 protein comprises a mRNA.

### Co-Stimulatory Molecules and Cytokines

In some aspects, enhanced APCs useful for the present disclosure further exhibit an increased expression of a co-stimulatory molecule as compared to corresponding APCs that have not been enhanced as described herein (“reference APCs”). In some aspects, enhanced APCs described herein further exhibit an increased expression of a cytokine as compared to the reference APCs. In some aspects, enhanced APCs described herein further exhibit an increased expression of both a co-stimulatory molecule and a cytokine as compared to the reference APCs.

As is generally understood in the art, for optimal T cell activation, multiple signals are required: (1) “signal 1”: antigen-specific signal provided by the binding of the TCR to antigenic peptide complexed with MHC; (2) “signal 2”: mediated by the engagement of co-stimulatory molecules such as CD80 and CD86 on antigen-presenting cells (APC); and (3) “signal 3”: mediated by cytokines (e.g., IL-2 and/or IL-12). Accordingly, compared to a reference formulation comprising corresponding APCs that have not been modified as described herein (e.g., does not exhibit increased expression of a co-stimulatory molecule and/or cytokine), a formulation comprising the enhanced APCs described herein is capable of inducing a much enhanced immune response. In some aspects, an enhanced immune response comprises: (i) an increase in the magnitude of the induced immune response as compared to that induced by the reference formulation, (ii) an increase in the breadth of the induced immune response as compared to that induced by the reference formulation, (iii) an increase in the duration of the induced immune response as compared to that induced by the reference formulation, or (iv) any combination of (i) to (iii).

In some aspects, the cytokine comprises a type I cytokine. In some aspects, the cytokine comprises IL-2, IL-4, IL-7, IL-10, IL-12, IL-15, IL-21, IL-la, IL-13, IL-Ira, IL-18, IL-33, IL-36α, IL-36β, IL-36γ, IL-36ra, IL-37, IL-38, IL-3, IL-5, IL-6, IL-11, IL-13, IL-23, granulocyte-macrophage colony stimulating factor (GM-CSF), granulocyte-colony stimulating factor (G-CSF), leukemia inhibitory factor (LIF), stem cell factor (SCF), thrombopoietin (TPO), macrophage-colony stimulating factor (M-CSF), erythropoieticn (EPO), Flt-3, IFN-α, IFN-β, IFN-γ, IL-19, IL-20, IL-22, IL-24, TNF-α, TNF-β, BAFF, APRIL, lymphotoxin beta (TNF-γ), IL-17A, IL-17B, IL-17C, IL-17D, IL-17E, IL-17F, IL-25, TSLP, IL-35, IL-27, TGF-β, or combinations thereof. In some aspects, the cytokine comprises IL-12. In some aspects, the cytokine comprises IL-2, In some aspects, the cytokine comprises both IL-12 and IL-2. In some aspects, the cytokine comprises a membrane bound form of the cytokine (“membrane-bound cytokine”). Amino acid sequences for exemplary membrane-bound cytokines are set forth in SEQ ID NOs: 7-10 and 13 (see Table 7 below).

In some aspects, the co-stimulatory molecule comprises OX40, OX40L, CD27, CD70, CD40, CD40L, 4-1BB, 4-1BBL, CD28, CD80, CD86, ICOS, ICOSL, or related molecules thereof, or any combination thereof. In some aspects, the co-stimulatory molecule comprises CD80. In some aspects, the co-stimulatory molecule comprises CD86.

Accordingly, as is apparent from at least the above disclosure, provided herein is a pharmaceutical composition comprising enhanced APCs which comprises a HPV antigen, wherein the enhanced APCs exhibit increased expression of a co-stimulatory molecule and/or a cytokine as compared to corresponding APCs that have not been modified (e.g., as described herein), and wherein the enhanced APCs are capable of activating T cells in a HLA-agnostic manner. More specifically, in some aspects, a pharmaceutical formulation comprises enhanced APCs which comprise a HPV E6 protein or variant thereof (e.g., full-length HPV-16 E6 protein) and a HPV E7 protein or variant thereof (e.g., full-length HPV-16 E7 protein), wherein the enhanced APCs exhibit increased expression of a co-stimulatory molecule (e.g., CD86) and a cytokine (e.g., membrane-bound IL-2 and membrane-bound IL-12) as compared to corresponding APCs that have not been modified (e.g., as described herein), and wherein the enhanced APCs are capable of activating T cells in a HLA-agnostic manner.

Not to be bound by any one theory, in some aspects, a nucleic acid encoding a co-stimulatory molecule and/or a nucleic acid encoding a cytokine can be introduced into APCs to modify them to exhibit increased expression of the co-stimulatory molecule and/or cytokine. In some aspects, such nucleic acids can be introduced into the APCs using a constriction-mediated delivery described herein. In some aspects, the enhanced cells described herein (e.g., comprising a HPV antigen) have been passed through a constriction under a set of parameters, thereby causing a perturbation within the APCs such that any of the following nucleic acids enter the APCs through the perturbation when contacted with the APCs: (i) a nucleic acid encoding a HPV E7 protein (e.g., full-length E7 protein of HPV-16), (ii) a nucleic acid encoding a HPV E6 protein (e.g., full-length E6 protein of HPV-16), (iii) a nucleic acid encoding a co-stimulatory molecule (e.g., CD86), (iv) a nucleic acid encoding a cytokine (e.g., membrane-bound IL-2 and/or membrane-bound IL-12), or (v) any combination of (i) to (iv). In some aspects, the nucleic acid comprises a mRNA.

In some aspects, any of the enhanced APCs provided herein (e.g., comprising a HPV antigen and/or exhibiting increased expression of a co-stimulatory molecule and/or cytokine) can be conditioned, such that APCs exhibit improved properties compared to corresponding APCs that have not been conditioned. As is apparent from the present disclosure, in some aspects, enhanced APCs described herein have been incubated in the presence of an adjuvant, such that the APCs are conditioned. In some aspects, the enhanced APCs described herein have been incubated with an adjuvant for about 2 hours to about 10 hours. In some aspects, the enhanced APCs described herein have been incubated with an adjuvant for about 3 hours to about 6 hours. In some aspects, the enhanced APCs described herein have been incubated with an adjuvant for about 4 hours. In some aspects, the incubation is done at about 37° C.

Non-limiting examples of adjuvants useful for the present disclosure comprises: stimulator of interferon genes (STING) agonists, retinoic acid-inducible gene I (RIG-I) agonists, and agonists for TLR3, TLR4, TLR7, TLR8, TLR9, CpG ODN, interferon-α (IFN-α), IFN-β, IFN-γ, alpha-galactosyl ceramide, polyinosinic:polycytidylic acid (polyL:C), imiquimod (R837), resiquimod (R848), cyclic dinucleotides (CDN), or lipopolysaccharide (LPS). In some aspects, the CpG ODN comprises a Class A CpG ODN, a Class B CpG ODN, or a Class C CpG ODN. In some aspects, the CpG ODN comprises CpG ODN 1018, CpG ODN 1585, CpG ODN 2216, CpG ODN 2336, CpG ODN 1668, CpG ODN 1826, CPG ODN 2006, CpG ODN 2007, CpG ODN BW006, CpG ODN D-SL01, CpG ODN 2395, CpG ODN M362, CpG ODN D-SL03.

### Cryopreservation Medium

As further described elsewhere in the present disclosure, in some aspects, the enhanced APCs provided herein are subsequently frozen (e.g., at cryogenic temperature). For instance, in some aspects, the enhanced APCs are frozen at temperatures at or below −100° C., −110° C., −120° C., −130° C., −140° C., −150° C., −160° C., −170° C., −180° C., −190° C., or −200° C. In some aspects, the enhanced APCs are frozen at temperature at or below −100° C. In some aspects, the enhanced APCs are frozen at temperature at or below −110° C. In some aspects, the enhanced APCs are frozen at temperature at or below −120° C. In some aspects, the enhanced APCs are frozen at temperature at or below −130° C. In some aspects, the enhanced APCs are frozen at temperature at or below −140° C. In some aspects, the enhanced APCs are frozen at temperature at or below −150° C. In some aspects, the enhanced APCs are frozen at temperature at or below −160° C. In some aspects, the enhanced APCs are frozen at temperature at or below −170° C. In some aspects, the enhanced APCs are frozen at temperature at or below −180° C. In some aspects, the enhanced APCs are frozen at temperature at or below −190° C. In some aspects, the enhanced APCs are frozen at temperature at or below −200° C.

In some aspects, a pharmaceutical formulation useful for the present disclosure comprises a cryopreservation medium. As used herein, the term “cryopreservation medium” refers to any compound that can be added to a sample to minimize or reduce damage to the cells (e.g., enhanced APCs) during freezing, thawing, and/or storage at temperatures below freezing. Accordingly, in some aspects, provided herein is a pharmaceutical formulation comprising: (a) enhanced APCs and (b) a cryopreservation medium, wherein the enhanced APCs are capable of activating T cells in a HLA-independent manner. In some aspects, a formulation provided herein comprises: (a) enhanced APCs which comprise a HPV antigen (e.g., HPV E6 protein and/or E7 protein) and (b) a cryopreservation medium, wherein the enhanced APCs are capable of activating T cells in a HLA-independent manner.

In some aspects, a formulation provided herein (e.g., comprising enhanced APCs and cryopreservation medium) is much more conducive to freeze-thaw cycles and/or long term storage at freezing conditions. For instance, in some aspects, compared to a reference formulation which lacks the cryopreservation medium (e.g., only comprises the enhanced APCs), greater percentage of the enhanced APCs remain viable after freeze-thaw cycles and/or long-term storage at freezing conditions. In some aspects, compared to the reference formulation, the percentage of viable cells after freeze-thaw cycles and/or long term storage at freezing conditions is increased by at least about 10%, at least about 20%, at least about 30%, at least about 40%, at least about 50%, at least about 60%, at least about 70%, at least about 80%, at least about 90%, or at least about 100%. In some aspects, compared to the reference formulation, the percentage of viable cells after freeze-thaw cycles and/or long term storage at freezing conditions is increased by at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, or at least about 50-fold.

In some aspects, the addition of the cryopreservation medium to the formulation does not significantly decrease the viability of the enhanced APCs. For instance, in some aspects, after the addition of the cryopreservation medium (and prior to any freezing), at least about 50%, at least about 60%, at least about 70%, at least about 80%, at least about 90%, or about 100% of the enhanced APCs are viable. The viability of cells can be assessed using any suitable methods known in the art (e.g., cell counting via hemocytometer and/or live/dead staining via flow cytometry). Additionally, in some aspects, after a freeze-thaw cycle and/or long-term storage in freezing conditions, at least about 50%, at least about 60%, at least about 70%, at least about 80%, at least about 90%, or about 100% of the enhanced APCs remain viable. In some aspects, at least about 70%, about 80%, about 90%, or about 95% of the enhanced APCs are viable after up to 1, 2, 3, 4, or 5 freeze-thaw cycles. In some aspects, at least about 70% of the enhanced APCs remain viable following storage for at least about 12 months at ≤−140° C.

In some aspects, the cryopreservation medium is present in the formulation at a concentration of about 20% to about 98% (w/w). In some aspects, the cryopreservation medium is at a concentration of about 20% (w/w), about 25% (w/w), about 30% (w/w), about 35% (w/w), about 40% (w/w), about 45% (w/w), about 50% (w/w), about 55% (w/w), about 60% (w/w), about 65% (w/w), about 70% (w/w), about 75% (w/w), about 80% (w/w), about 85% (w/w), about 90% (w/w), about 95% (w/w), or about 98% (w/w). In some aspects, of the cryopreservation medium is at a concentration of about 20% to about 25% (w/w), about 25% to 30% (w/w), about 30% to 35% (w/w), about 35% to 40% (w/w), about 40% to 45% (w/w), about 45% to 50% (w/w), about 50% to 55% (w/w), about 55% to 60% (w/w), about 60% to 65% (w/w), about 65% to 70% (w/w), about 70% to 75% (w/w), about 75% to 80% (w/w), about 80% to 85% (w/w), about 85% to 90% (w/w), or about 90% to 95% (w/w). In some aspects, the cryopreservation medium is at a concentration of about 40% to about 95% (w/w). In some aspects, the cryopreservation medium is present at a concentration of about 50% (w/w).

Accordingly, in some aspects, provided herein is a pharmaceutical formulation comprising: (a) enhanced APCs and (b) a cryopreservation medium; wherein the cryopreservation medium is at a concentration of about 40% to about 95% (w/w), and wherein the enhanced APCs are capable of activating T cells in a HLA-independent manner. In some aspects, a pharmaceutical formulation useful for the present disclosure comprises: (a) enhanced APCs comprising a HPV antigen (e.g., HPV E6 protein and/or E7 protein) and (b) a cryopreservation medium; wherein the cryopreservation medium is at a concentration of about 50% (w/w), and wherein the enhanced APCs are capable of activating T cells in a HLA-independent manner.

In some aspects, any suitable cryopreservation medium known in the art can be used with the present disclosure. Non-limiting examples of cryopreservation medium includes: a disaccharide, such as sucrose or trehalose, dimethylsulfoxide (DMSO), hydroxyethyl starch, glycerol, polyethylene glycol, polyvinylpyrrolidone, methylcellulose, proline, a polymer, ectoin, dextran, hypothermosol, plasmalyte, human serum albumin, human serum, and combinations thereof. Cryoprotectants are known in the art and described further, e.g., in Janz et al., Journal of Biomedicine and Biotechnology 2012; Mareschi et al. Experimental Hematology 2006 34:1563-1572; and Hunt et al. Transfus Med Hemother 2011 38:107-123, each of which is incorporated by reference herein in its entirety. In some aspects, the cryopreservation medium is CryoStor® CS10.

CryoStor® CS10 (BioLife Solution) is a serum-free, protein-free, defined cryopreservation medium containing 10% DMSO that is used as a cryoprotectant, an osmolality agent, and for pH control. CryoStor® CS10 is pre-formulated with DMSO, a cryoprotective agent which helps mitigate cell damage from the formation of intracellular ice. Accordingly, in some aspects, a cryopreservation medium useful for the present disclosure comprises DMSO.

In some aspects, the cryopreservation medium comprises about 2% to about 25% DMSO. In some aspects, the cryopreservation medium comprises about 5% to about 15% DMSO. In some aspects, the cryopreservation medium comprises about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 23%, about 24%, or about 25% DMSO. In some aspects, the cryopreservation medium comprises about 2% to about 5%, about 5% to about 6%, about 6% to about 7%, about 7% to about 8%, about 8% to about 9%, about 9% to about 10%, about 10% to about 11%, about 11% to about 12%, about 12% to about 13%, about 13% to about 14%, about 14% to about 15%, or about 15% to about 20% DMSO. In some aspects, the cryopreservation medium comprises about 10% DMSO.

### Hypothermic Preservation Medium

In some aspects, a pharmaceutical formulation described herein comprises a hypothermic preservation medium. As used herein, the term “hypothermic preservation medium” refers to any compounds that can help improve and/or extend the preservation of cells (e.g., enhanced APCs described herein) particularly at non-freezing temperatures (e.g., 2-8° C.). Therefore, in some aspects, provided herein is a pharmaceutical formulation comprising: (a) enhanced APCs and (b) a hypothermic preservation medium, wherein the enhanced APCs are capable of activating T cells in a HLA-independent manner. In some aspects, a formulation described herein comprises: (a) enhanced APCs, (b) a cryopreservation medium, and (c) a hypothermic preservation medium; wherein the enhanced APCs are capable of activating T cells in a HLA-independent manner. In some aspects, a formulation provided herein comprises: (a) enhanced APCs which comprise a HPV antigen (e.g., HPV E6 protein and/or E7 protein), (b) cryopreservation medium, and (c) a hypothermic preservation medium; wherein the enhanced APCs are capable of activating T cells in a HLA-independent manner.

In some aspects, the presence of both the cryopreservation medium and the hypothermic preservation medium further improves the viability of the enhanced APCs. In some aspects, compared to a reference formulation which either lacks the cryopreservation medium or the hypothermic preservation medium, the viability of the enhanced APCs is increased. In some aspects, the viability is increased by at least about 10%, at least about 20%, at least about 30%, at least about 40%, at least about 50%, at least about 60%, at least about 70%, at least about 80%, at least about 90%, or at least about 100%, as compared to the corresponding cells of the reference formulation. In some aspects, the viability is increased by at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, or at least about 50-fold, as compared to the corresponding cells of the reference formulation.

In some aspects, the addition of the hypothermic preservation medium to the formulation does not significantly decrease the viability of the enhanced APCs. Accordingly, in some aspects, the addition of both the cryopreservation medium and the hypothermic preservation medium does not significantly decrease the viability of the enhanced APCs. In some aspects, after the addition of the hypothermic preservation medium (alone or in combination with a cryopreservation medium), at least about 50%, at least about 60%, at least about 70%, at least about 80%, at least about 90%, or about 100% of the enhanced APCs are viable. Additionally, in some aspects, after a freeze-thaw cycle and/or long-term storage in freezing conditions, at least about 50%, at least about 60%, at least about 70%, at least about 80%, at least about 90%, or about 100% of the enhanced APCs remain viable. In some aspects, at least about 70%, about 80%, about 90%, or about 95% of the enhanced APCs are viable after up to 1, 2, 3, 4, or 5 freeze-thaw cycles. In some aspects, at least about 70% of the enhanced APCs remain viable following storage for at least about 12 months at ≤−140° C.

In some aspects, the hypothermic preservation medium is present in the formulation at a concentration of about 10% to about 70% (w/w). In some aspects, the hypothermic preservation medium is at a concentration of about of about 10% (w/w), about 15% (w/w), about 20% (w/w), about 25% (w/w), about 30% (w/w), about 35% (w/w), about 40% (w/w), about 45% (w/w), about 50% (w/w), about 55% (w/w), about 60% (w/w), about 65% (w/w), or about 70% (w/w). In some aspects, the hypothermic preservation medium is at a concentration of about 10% to about 15% (w/w), about 15% to about 20% (w/w), about 20% to about 25% (w/w), about 25% to about 30% (w/w), about 30% to about 35% (w/w), about 35% to about 40% (w/w), about 40% to about 45% (w/w), about 45% to about 50% (w/w), about 50% to about 55% (w/w), about 55% to about 60% (w/w), about 60% to about 65% (w/w), or about 65% to about 70% (w/w). In some aspects, the hypothermic preservation medium is at a concentration of about 25% to about 35% (w/w). In some aspects, the hypothermic preservative medium is at a concentration of about 30% (w/w).

In some aspects, provided herein is a pharmaceutical formulation comprising: (a) enhanced APCs and (b) a hypothermic preservative medium; wherein the hypothermic preservative medium is at a concentration of about 25% to about 35% (w/w), and wherein the enhanced APCs are capable of activating T cells in a HLA-independent manner. In some aspects, a pharmaceutical formulation useful for the present disclosure comprises: (a) enhanced APCs comprising a HPV antigen (e.g., HPV E6 protein and/or E7 protein) and (b) a hypothermic preservative medium; wherein the hypothermic preservative medium is at a concentration of about 50% (w/w), and wherein the enhanced APCs are capable of activating T cells in a HLA-independent manner. In some aspects, provided herein is a pharmaceutical formulation comprising: (a) enhanced APCs, (b) a cryopreservation medium, and (c) a hypothermic preservative medium; wherein the cryopreservation medium is at a concentration of about 40% to about 95% (w/w), wherein the hypothermic preservative medium is at a concentration of about 25% to about 35% (w/w), and wherein the enhanced APCs are capable of activating T cells in a HLA-independent manner. In some aspects, a pharmaceutical formulation useful for the present disclosure comprises: (a) enhanced APCs comprising a HPV antigen (e.g., HPV E6 protein and/or E7 protein), (b) a cryopreservation medium, and (c) a hypothermic preservative medium; wherein the cryopreservation medium is at a concentration of about 50% (w/w), wherein the hypothermic preservative medium is at a concentration of about 30% (w/w), and wherein the enhanced APCs are capable of activating T cells in a HLA-independent manner.

In some aspects, any suitable hypothermic preservative medium known in the art can be used. In some aspects, the hypothermic preservative medium comprises HypoThermasol® FRS. HypoThermosol® FRS (BioLife Solution) is a serum-free, protein-free, DMSO-free hypothermic preservation medium that is similar in composition to CryoStor® CS10. The difference is that CryoStor® CS10 contains DMSO which is replaced with Trolox (a water-soluble analog of Vitamin E) for HypoThermosol® FRS. Accordingly, in some aspects, the hypothermic preservation medium comprises a water soluble analog of vitamin E. In some aspects, the hypothermic preservation medium comprises trolox ((±)-6-hydroxy-2,5,7,8-tetramethylchromane-2-carboxylic acid).

### Additional Components

In some aspects, a pharmaceutical formulation provided herein further comprises one or more additional components. In some aspects, the one or more additional components can improve the function of the other elements included in the formulation (e.g., cryopreservation medium and/or hypothermic preservation medium). In some aspects, the one or more additional components can improve one or more properties of the formulations. Non-limiting examples of such properties include: reduce surface adsorption, reduce aggregation, reduce fibrillation, reduce oxidation, improve solubility, improve lyophilization, or combinations thereof. In some aspects, the one or additional components enhance endocytosis, improves the stability of the formulation, or both. Non-limiting examples of additional components that can be included in a formulation described herein include: a divalent metal cation, glucose, ATP, potassium, glycerol, trehalose, D-sucrose, PEG1500, L-arginine, L-glutamine, or EDTA. In some aspects, the divalent metal cation comprises one or more of Mg2+, Zn2+ or Ca2+. In some aspects, the one or more additional components comprise sodium pyruvate, adenine, trehalose, dextrose, mannose, sucrose, human serum albumin (HSA), HEPES, glycerol, glutathione, inosine, dibasic sodium phosphate, monobasic sodium phosphate, sodium metal ions, potassium metal ions, magnesium metal ions, chloride, acetate, gluoconate, sucrose, potassium hydroxide, or sodium hydroxide. In some aspects, the one or more additional components comprise Sodium pyruvate, adenine, Rejuvesol®, trehalose, dextrose, mannose, sucrose, human serum albumin (HSA), PlasmaLyte®, Cryostor® CS2, Cryostor® CS5, Cryostor® CS15, HEPES, glycerol, or glutathione.

In some aspects, such additional component comprises human serum albumin. Unless indicated otherwise, in some aspects, human serum albumin can be substituted with albumin from a different source (e.g., mouse or bovine). Accordingly, in some aspects, provided herein is a pharmaceutical formulation comprising: (a) enhanced APCs and (b) human serum albumin, wherein the enhanced APCs are capable of activating T cells in a HLA-independent manner. In some aspects, a formulation described herein comprises: (a) enhanced APCs, (b) a cryopreservation medium, (c) a hypothermic preservation medium, and (d) human serum albumin; wherein the enhanced APCs are capable of activating T cells in a HLA-independent manner. In some aspects, a formulation provided herein comprises: (a) enhanced APCs which comprise a HPV antigen (e.g., HPV E6 protein and/or E7 protein), (b) cryopreservation medium, (c) a hypothermic preservation medium, and (d) human serum albumin; wherein the enhanced APCs are capable of activating T cells in a HLA-independent manner.

In some aspects, the human serum albumin is provided in a human serum albumin solution. In some aspects, the albumin solution is an Albumin (Human), USP, 25% Solution.

Albumin (Human), USP, 25% Solution is a sterile preparation of albumin for intravenous administration. Albumin (Human) is a 25% sterile solution of albumin in an aqueous diluent. The preparation is stabilized with sodium caprylate (about 0.08 mmol/g albumin) and acetyltryptophan (about 0.08 mmol/g albumin). It is a clear, slightly viscous liquid, which can range from almost colorless, to yellow, amber or green. In some aspects, the albumin solution contains no preservative.

In some aspects, the human serum albumin solution comprises about 10%, about 15%, about 20%, about 25%, about 30%, about 35%, or about 40%. In some aspects, the human serum albumin solution comprises about 25% human serum albumin (“25% human serum albumin solution”). In some aspects, the formulation comprises human serum albumin solution at a concentration of about any one of 2%, 3%, 4%, 5%, 8%, 10%, 15%, 20%, 25%, 30%, 35%, 40%, 45%, or 50% (w/w). In some aspects, the percentage of a human serum albumin solution in the formulation is about any one of 2% to 3%, 3% to 5%, 5% to 8%, 8% to 10%, 10% to 15%, 15% to 20%, 20% to 25%, 5% to 30%, 30% to 35%, 5% to 40%, 40% to 45%, or 45% to 50% (w/w). In some aspects, the percentage of a human serum albumin solution in the formulation is about 15% to about 25% (w/w). In some aspects, the percentage of a human serum albumin solution in the formulation is about is about 20% (w/w). In some aspects, the human albumin solution comprises sodium caprylate at a concentration of about 0.001, about 0.01, about 0.02, about 0.03, about 0.04, about 0.05, about 0.06, about 0.07, about 0.08, about 0.09, about 0.1, about 0.2, about 0.5, or about 1 mmol/g of albumin. In some aspects, the human albumin solution comprises sodium caprylate at a concentration of about 0.08 mmol/g of albumin. In some aspects, the human albumin solution comprises acetyltryptophan at a concentration of about 0.001, about 0.01, about 0.02, about 0.03, about 0.04, about 0.05, about 0.06, about 0.07, about 0.08, about 0.09, about 0.1, about 0.2, about 0.5, or about 1 mmol/g of albumin. In some aspects, the human albumin solution comprises acetyltryptophan at a concentration of about 0.08 mmol/g of albumin.

In some aspects, a pharmaceutical formulation provided herein comprises: (a) enhanced APCs and (b) a 25% human serum albumin solution at a concentration of about 15% to about 25% (w/w), wherein the enhanced APCs are capable of activating T cells in a HLA-agnostic manner. In some aspects, a pharmaceutical formulation provided herein comprises: (a) enhanced APCs, (b) a cryopreservation medium at a concentration of about 40% to about 95% (w/w), and (c) a 25% human serum albumin solution at a concentration of about 15% to about 25% (w/w); wherein the enhanced APCs are capable of activating T cells in a HLA-agnostic manner. In some aspects, a pharmaceutical formulation provided herein comprises: (a) enhanced APCs, (b) a hypothermic preservation medium at a concentration of about 25% to about 35% (w/w), and (c) a 25% human serum albumin solution at a concentration of about 15% to about 25% (w/w); wherein the enhanced APCs are capable of activating T cells in a HLA-agnostic manner. In some aspects, a pharmaceutical formulation provided herein comprises: (a) enhanced APCs, (b) a cryopreservation medium at a concentration of about 40% to about 95% (w/w), (c) a hypothermic preservation medium at a concentration of about 25% to about 35% (w/w), and (d) a 25% human serum albumin solution at a concentration of about 15% to about 25% (w/w); wherein the enhanced APCs are capable of activating T cells in a HLA-agnostic manner. In some aspects, a pharmaceutical formulation provided herein comprises: (a) enhanced APCs comprising a HPV antigen (e.g., HPV E6 and/or E7 protein), (b) a cryopreservation medium at a concentration of about 50% (w/w), (c) a hypothermic preservation medium at a concentration of about 30% (w/w), and (d) a 25% human serum albumin solution at a concentration of about 20% (w/w); wherein the enhanced APCs are capable of activating T cells in a HLA-independent manner.

### Fill Volume

As described herein, in some aspects, a pharmaceutical formulation described herein is a liquid formulation. Any suitable liquid can be used to produce such a liquid formulation. In some aspects, each of the various components described herein (e.g., enhanced APCs, cryopreservation medium, hypothermic preservation medium, and human serum albumin) are combined in a suitable liquid medium, such that the total volume of the formulation is about 2 mL to about 150 mL. In some aspects, the fill volume (i.e., the liquid volume in which the other components of the formulation are resuspended in) of the formulation is about 1, about 2, about 3, about 4, about 5, about 6, about 7, about 8, about 9, about 9.5, about 10, about 10.5, about 11, about 12, about 13, about 14, about 15, about 16, about 17, about 18, about 19, about 20, about 25, about 30, about 35, about 40, about 45, about 50, about 60, about 70, about 80, about 90, about 100, about 110, about 120, about 130, about 140, or about 150 mL or more. In some aspects, the fill volume is about 1 to about 2, about 2 to about 3, about 3 to about 4, about 4 to about 5, about 5 to about 6, about 6 to about 7, about 7 to about 8, about 8 to about 9, or about 9 to about 10, about 10 to about 11, about 11 to about 12, about 12 to about 13, about 13 to about 14, about 14 to about 15, about 15 to about 16, about 16 to about 17, about 17 to about 18, about 18 to about 19, or about 19 to about 20 mL. In some aspects, the fill volume of the formulation is about 10 mL. In some aspects, the fill volume is about 9.5 mL. In some aspects, the volume of the formulation is about 5 mL.

pH

For any of the pharmaceutical formulations described herein, in some aspects, the formulation has a specific pH. In some aspects, the pH of the formulation enhances and/or improves one or more properties of the formulation (e.g., solubility, stability, tolerability, and/or activity). In some aspects, the pH of the formulation is about 5.0 to about 9.5. In some aspects, the pH of the formulation is about 6.0 to about 8.5. In some aspects, the pH of the formulation is about 7.0 to about 7.9. In some aspects, the pH of the formulation is about 7.9. In some aspects, of the formulation has a pH of about 5, about 5.5, about 6, about 6.5, about 7, about 7.5, about 8, about 8.5, about 9, or about 10. In some aspects, the formulation has a pH of about 7, about 7.1, about 7.2, about 7.3, about 7.4, about 7.5, about 7.6, about 7.7, about 7.8, about 7.9, or about 8.0. In some aspects, the pH of the formulation is about 5 to about 6, about 6 to about 7, about 7 to about 8, about 8 to about 9, or about 9 to about 10. In some aspects, the pH of the formulation is about 7 to about 7.1, about 7.1 to about 7.2, about 7.2 to about 7.3, about 7.3 to about 7.4, about 7.4 to about 7.5, about 7.5 to about 7.6, about 7.6 to about 7.7, about 7.7 to about 7.8, about 7.8 to about 7.9, or about 7.9 to about 8.0.

As is apparent from at least the above disclosures, in some aspects, provided herein is a pharmaceutical formulation comprising: (a) about 5×10⁶ enhanced antigen presenting cells (“enhanced APCs”) to about 1×10⁹ enhanced APCs; (b) a cryopreservation medium (e.g., CryoStor® CS10) at a concentration of about 40% (w/w) to about 95% (w/w); (c) a solution comprising about 25% human serum albumin (“human serum albumin solution”) at a concentration of about 15% (w/w) to about 25% (w/w); wherein the pH of the formulation is about 6.0 to about 8.5, and wherein the enhanced APCs are capable of activating T cells in an HLA agnostic manner. In some aspects, provided herein is a pharmaceutical formulation comprising: (a) enhanced antigen present cells (“enhanced APCs”) at a concentration of about 1.1×10⁷ enhanced APCs/mL; (b) a cryopreservation medium (e.g., CryoStor® CS10) at a concentration of about 50% (w/w); (c) a solution comprising about 25% human serum albumin (“human serum albumin solution”) at a concentration of about 20% (w/w); wherein the pH of the formulation is about 7.0 to about 7.9, and wherein the enhanced APCs are capable of activating T cells in an HLA agnostic manner. In some aspects, provided herein is a pharmaceutical formulation comprising: (a) enhanced antigen present cells (“enhanced APCs”) at a concentration of about 5×10⁶ enhanced APCs to about 1×10⁹ enhanced APCs; (b) a cryopreservation medium (e.g., CryoStor® CS10) at a concentration of about 40% (w/w) to about 95% (w/w); (c) a hypothermic preservation medium (e.g., HypoThermasol® FRS) at a concentration of about 25% (w/w) to about 35% (w/w); (d) a solution comprising about 25% human serum albumin (“human serum albumin solution”) at a concentration of about 15% (w/w) to about 25% (w/w); wherein the pH of the formulation is about 6.0 to about 8.5, and wherein the enhanced APCs are capable of activating T cells in an HLA agnostic manner. In some aspects, provided herein is a pharmaceutical formulation comprising: (a) enhanced antigen present cells (“enhanced APCs”) at a concentration of about 1.1×10⁷ enhanced APCs/mL; (b) a cryopreservation medium (e.g., CryoStor® CS10) at a concentration of about 50% (w/w); (d) a hypothermic preservation medium (e.g., HypoThermasol® FRS) at a concentration of about 30% (w/w); (c) a solution comprising about 25% human serum albumin (“human serum albumin solution”) at a concentration of about 20% (w/w); wherein the pH of the formulation is about 7.0 to about 7.9, and wherein the enhanced APCs are capable of activating T cells in an HLA agnostic manner. In some aspects, provided herein is a pharmaceutical formulation comprising: (a) about 1.05×10⁸ enhanced APCs, (b) about 4.99 g of a cryopreservation medium (e.g., CryoStor® CS10), (c) about 2.00 g of a solution comprising 25% human serum albumin (“human serum albumin solution”); wherein the pH of the formulation is about 7.0 to about 7.9; and wherein the enhanced APCs are capable of activating T cells in an agnostic manner. In some aspects, provided herein is a pharmaceutical formulation comprising: (a) about 1.05×10⁸ enhanced APCs, (b) about 4.99 g of a cryopreservation medium (e.g., CryoStor® CS10), (c) about 2.99 g of a hypothermic preservation medium (e.g., HypoThermasol® FRS), (d) about 2.00 g of a solution comprising 25% human serum albumin (“human serum albumin solution”); wherein the pH of the formulation is about 7.0 to about 7.9; and wherein the enhanced APCs are capable of activating T cells in an agnostic manner.

In some aspects, the enhanced APCs in the formulation maintain equal to or greater than about 50% viability up to 1, 2, 3, 4, 5 freeze-thaw cycles. In some aspects, the enhanced APCs in the formulation maintain at least about 50%, at least about 60%, at least about 70%, at least about 80%, at least about 90%, at least about 95%, or at least about 99% viability up to 1, 2, 3, 4, 5 freeze-thaw cycles. In some aspects, the enhanced APCs in the formulation maintain at least about 70% viability following storage for at least 12 months at temperatures at or below −140° C. In some aspects, the enhanced APCs maintain at least about 50%, at least about 60%, at least about 70%, at least about 80%, at least about 90%, at least about 95%, or at least about 99% viability following storage for at least 12 months at temperatures at or below −140° C. In some aspects, the enhanced APCs in the formulation maintain at least about 70% viability following storage for at least 3, 6, 9, 12, 15, 18, 24, 30, or 36 months at temperatures at or below −140° C. In some aspects, the enhanced APCs in the formulation maintain at least about 70% viability following storage for at least 3 months at temperatures at or below −100° C., −110° C., −120° C., −130° C., −140° C., −150° C., −160° C., −170° C., −180° C., −190° C., or −200° C. In some aspects, the enhanced APCs in the formulation maintain at least about 70% viability following storage for at least 12 months at temperatures at or below −100° C., −110° C., −120° C., −130° C., −140° C., −150° C., −160° C., −170° C., −180° C., −190° C., or −200° C.

Pharmaceutical formulations described herein are suitable for in vivo administration. Accordingly, in some aspects, a formulation provided herein is sterile. In some aspects, the formulation comprise less than about 2 EU/mL endotoxin. In some aspects, the formulation comprise less than about 1, less than about 2, less than about 3, less than about 4, less than about 5, less than about 6, less than about 7, less than about 8, less than about 9, or less than about 10 EU/mL endotoxin. In some aspects, the formulation is free of mycoplasma.

### Vials Comprising Pharmaceutical Formulations

In some aspects, the present disclosure relates to vials comprising any of the pharmaceutical formulations provided herein.

Accordingly, in some aspects, present disclosure provides a vial comprising a pharmaceutical formulation, which comprises: (a) enhanced APCs that are capable of activating T cells in a HLA-agnostic manner and (b) a cryopreservation medium. In some aspects, provided herein is a vial comprising a pharmaceutical formulation, which comprises (a) enhanced APCs that are capable of activating T cells in a HLA-agnostic manner and (b) a hypothermic preservation medium. In some aspects, provided herein is a vial comprising a pharmaceutical formulation, which comprises (a) enhanced APCs that are capable of activating T cells in a HLA-agnostic manner and (b) human serum albumin. In some aspects, the present disclosure provides a vial comprising a pharmaceutical formulation, which comprises: (a) enhanced APCs that are capable of activating T cells in a HLA-agnostic manner, (b) a cryopreservation medium, (c) a hypothermic preservation medium, and (d) human serum albumin.

More specifically, in some aspects, provided herein is a vial comprising a pharmaceutical formulation, which comprises: (a) about 5×10⁶ enhanced antigen presenting cells (“enhanced APCs”) to about 1×10⁹ enhanced APCs; (b) a cryopreservation medium (e.g., CryoStor® CS10) at a concentration of about 40% (w/w) to about 95% (w/w); (c) a solution comprising about 25% human serum albumin (“human serum albumin solution”) at a concentration of about 15% (w/w) to about 25% (w/w); wherein the pH of the formulation is about 6.0 to about 8.5, and wherein the enhanced APCs are capable of activating T cells in an HLA agnostic manner. In some aspects, provided herein is a vial comprising a pharmaceutical formulation, which comprises: (a) enhanced antigen present cells (“enhanced APCs”) at a concentration of about 1.1×10⁷ enhanced APCs/mL; (b) a cryopreservation medium (e.g., CryoStor® CS10) at a concentration of about 50% (w/w); (c) a solution comprising about 25% human serum albumin (“human serum albumin solution”) at a concentration of about 20% (w/w); wherein the pH of the formulation is about 7.0 to about 7.9, and wherein the enhanced APCs are capable of activating T cells in an HLA agnostic manner. In some aspects, provided herein is a vial comprising a pharmaceutical formulation, which comprises: (a) enhanced antigen present cells (“enhanced APCs”) at a concentration of about 5×10⁶ enhanced APCs to about 1×10⁹ enhanced APCs; (b) a cryopreservation medium (e.g., CryoStor® CS10) at a concentration of about 40% (w/w) to about 95% (w/w); (c) a hypothermic preservation medium (e.g., HypoThermasol® FRS) at a concentration of about 25% (w/w) to about 35% (w/w); (d) a solution comprising about 25% human serum albumin (“human serum albumin solution”) at a concentration of about 15% (w/w) to about 25% (w/w); wherein the pH of the formulation is about 6.0 to about 8.5, and wherein the enhanced APCs are capable of activating T cells in an HLA agnostic manner. In some aspects, provided herein is a vial comprising a pharmaceutical formulation, which comprises: (a) enhanced antigen present cells (“enhanced APCs”) at a concentration of about 1.1×10⁷ enhanced APCs/mL; (b) a cryopreservation medium (e.g., CryoStor® CS10) at a concentration of about 50% (w/w); (d) a hypothermic preservation medium (e.g., HypoThermasol® FRS) at a concentration of about 30% (w/w); (c) a solution comprising about 25% human serum albumin (“human serum albumin solution”) at a concentration of about 20% (w/w); wherein the pH of the formulation is about 7.0 to about 7.9, and wherein the enhanced APCs are capable of activating T cells in an HLA agnostic manner. In some aspects, provided herein is a vial comprising a pharmaceutical formulation, which comprises: (a) enhanced antigen present cells (“enhanced APCs”) at a concentration of about 8.5×10⁶. enhanced APCs/mL; (b) a cryopreservation medium (e.g., CryoStor® CS10) at a concentration of about 50% (w/w); (d) a hypothermic preservation medium (e.g., HypoThermasol® FRS) at a concentration of about 30% (w/w); (c) a solution comprising about 25% human serum albumin (“human serum albumin solution”) at a concentration of about 20% (w/w); wherein the pH of the formulation is about 7.0 to about 7.9, and wherein the enhanced APCs are capable of activating T cells in an HLA agnostic manner. In some aspects, provided herein is a vial comprising a pharmaceutical formulation, which comprises: (a) about 1.05×10⁸ enhanced APCs, (b) about 4.99 g of a cryopreservation medium (e.g., CryoStor® CS10), (c) about 2.00 g of a solution comprising 25% human serum albumin (“human serum albumin solution”); wherein the pH of the formulation is about 7.0 to about 7.9; and wherein the enhanced APCs are capable of activating T cells in an agnostic manner. In some aspects, provided herein is a vial comprising a pharmaceutical formulation, which comprises: (a) about 1.05×10⁸ enhanced APCs, (b) about 4.99 g of a cryopreservation medium (e.g., CryoStor® CS10), (c) about 2.99 g of a hypothermic preservation medium (e.g., HypoThermasol® FRS), (d) about 2.00 g of a solution comprising 25% human serum albumin (“human serum albumin solution”); wherein the pH of the formulation is about 7.0 to about 7.9; and wherein the enhanced APCs are capable of activating T cells in an agnostic manner.

In some aspects, the vial comprises about 2 mL to about 50 mL of a cryopreservation medium. In some aspects, in the vial comprises about 4 mL to about 20 mL of cryopreservation medium. In some aspects, the vial comprises about 1, about 2, about 3, about 4, about 5, about 6, about 7, about 8, about 9, about 9.5, about 10, about 10.5, about 11, about 12, about 13, about 14, about 15, about 16, about 17, about 18, about 19, about 20, about 25, about 30, about 35, about 40, about 45, or about 50 mL of cryopreservation medium. In some aspects, the vial comprises about 1 to about 2, about 2 to about 3, about 3 to about 4, about 4 to about 5, about 5 to about 6, about 6 to about 7, about 7 to about 8, about 8 to about 9, or about 9 to about 10, about 10 to about 11, about 11 to about 12, about 12 to about 13, about 13 to about 14, about 14 to about 15, about 15 to about 16, about 16 to about 17, about 17 to about 18, about 18 to about 19, or about 19 to about 20 mL of cryopreservation medium. In some aspects, the vial comprises about 4 to about 5 mL of a cryopreservation medium. In some aspects, the vial comprises about 4.45 mL of a cryopreservation medium.

In some aspects, the vial comprises about 1 mL to about 50 mL of a hypothermic preservation medium. In some aspects, the vial comprises about 2 mL to about 20 mL of a hypothermic preservation medium. In some aspects, the vial comprises about 1, about 2, about 3, about 4, about 5, about 6, about 7, about 8, about 9, about 9.5, about 10, about 10.5, about 11, about 12, about 13, about 14, about 15, about 16, about 17, about 18, about 19, about 20, about 25, about 30, about 35, about 40, about 45, or about 50 mL of a hypothermic preservation medium. In some aspects, the vial comprises about 1 to about 2, about 2 to about 3, about 3 to about 4, about 4 to about 5, about 5 to about 6, about 6 to about 7, about 7 to about 8, about 8 to about 9, or about 9 to about 10, about 10 to about 11, about 11 to about 12, about 12 to about 13, about 13 to about 14, about 14 to about 15, about 15 to about 16, about 16 to about 17, about 17 to about 18, about 18 to about 19, or about 19 to about 20 mL of a hypothermic preservation medium. In some aspects, the vial comprises about 2 to 3 mL of a hypothermic preservation medium. In some aspects, the vial comprises about 2.67 mL of a cryopreservation medium. In some aspects, the vial comprises: (i) about 5×10⁶ enhanced APCs to about 1×10⁹ enhanced APCs; (ii) about 4 to about 5 mL of a cryopreservation medium (e.g., CryoStor® CS10); and (iii) about 2 to 3 mL of a hypothermic preservation medium (e.g., HypoThermasol®). In some aspects, the vial comprises: (i) about 7×10⁷ enhanced APCs to about 8×10⁷ enhanced APCs (e.g., about 7.6×10⁷ enhanced APCs); (ii) about 4.45 mL of a cryopreservation medium (e.g., CryoStor® CS10); and (iii) about 2.67 mL of a hypothermic preservation medium (e.g., HypoThermasol®).

In some aspects, the vial comprises human serum albumin. In some aspects, the vial comprises about 1 to about 10 mL of a human serum albumin solution. In some aspects, the vial comprises about 1, about 2, about 3, about 4, about 5, about 6, about 7, about 8, about 9, about 9.5, or about 10 mL of a human serum albumin solution. In some aspects, the vial comprises about 1 to about 2 mL, about 2 to about 3 mL, about 3 to about 4 mL, about 4 to about 5 mL, about 5 to about 6 mL, about 6 to about 7 mL, about 7 to about 8 mL, about 8 to about 9 mL, or about 9 to about 10 mL of a human serum albumin solution. In some aspects, the vial comprises about 1 to about 2 mL of a human serum albumin solution. In some aspects, the vial comprises about 1.78 mL of a human serum albumin solution.

In some aspects, the vial comprises: (i) about 5×10⁶ enhanced APCs to about 1×10⁹ enhanced APCs; (ii) about 4 to about 5 mL of a cryopreservation medium (e.g., CryoStor® CS10); (iii) about 2 to 3 mL of a hypothermic preservation medium (e.g., HypoThermasol®), and (iv) about 1 to about 2 mL of a human serum albumin solution. In some aspects, the vial comprises: (i) about 7×10⁷ enhanced APCs to about 8×10⁷ enhanced APCs (e.g., about 7.6×10⁷ enhanced APCs); (ii) about 4.45 mL of a cryopreservation medium (e.g., CryoStor® CS10); (iii) about 2.67 mL of a hypothermic preservation medium (e.g., HypoThermasol®), and about 1.78 mL of a human serum albumin solution.

### Methods of Producing a Pharmaceutical Formulation

Some aspects of the present disclosure relates to methods of producing any of the pharmaceutical formulations described herein. In some aspects, such a method comprises combining enhanced APCs which are capable of activating T cells in a HLA-agnostic manner with one or more of the following: a cryopreservation medium, a hypothermic preservation medium, and a human serum albumin. In some aspects, the method comprises combining the enhanced APCs with a cryopreservation medium. In some aspects, the method comprises combining the enhanced APCs with a hypothermic preservation medium. In some aspects, the method comprises combining the enhanced APCs with a human serum albumin. In some aspects, the method comprises combining the enhanced APCs with a cryopreservation medium, hypothermic preservation medium, and a human serum albumin.

In some aspects, the method comprises combining: (a) about 5×10⁶ enhanced antigen presenting cells (“enhanced APCs”) to about 1×10⁹ enhanced APCs; (b) a cryopreservation medium (e.g., CryoStor® CS10) at a concentration of about 40% (w/w) to about 95% (w/w); and (c) a solution comprising about 25% human serum albumin (“human serum albumin solution”) at a concentration of about 15% (w/w) to about 25% (w/w). In some aspects, the method comprises combining: (a) enhanced antigen present cells (“enhanced APCs”) at a concentration of about 1.1×10⁷ enhanced APCs/mL; (b) a cryopreservation medium (e.g., CryoStor® CS10) at a concentration of about 50% (w/w); and (c) a solution comprising about 25% human serum albumin (“human serum albumin solution”) at a concentration of about 20% (w/w). In some aspects, method comprises combining: (a) enhanced antigen present cells (“enhanced APCs”) at a concentration of about 5×10⁶ enhanced APCs to about 1×10⁹ enhanced APCs; (b) a cryopreservation medium (e.g., CryoStor® CS10) at a concentration of about 40% (w/w) to about 95% (w/w); (c) a hypothermic preservation medium (e.g., HypoThermasol® FRS) at a concentration of about 25% (w/w) to about 35% (w/w); and (d) a solution comprising about 25% human serum albumin (“human serum albumin solution”) at a concentration of about 15% (w/w) to about 25% (w/w). In some aspects, method comprises combining: (a) enhanced antigen present cells (“enhanced APCs”) at a concentration of about 1.1×10⁷ enhanced APCs/mL; (b) a cryopreservation medium (e.g., CryoStor® CS10) at a concentration of about 50% (w/w); (d) a hypothermic preservation medium (e.g., HypoThermasol® FRS) at a concentration of about 30% (w/w); (c) a solution comprising about 25% human serum albumin (“human serum albumin solution”) at a concentration of about 20% (w/w). In some aspects, method comprises combining: (a) about 1.05×10⁸ enhanced APCs, (b) about 4.99 g of a cryopreservation medium (e.g., CryoStor® CS10), (c) about 2.00 g of a solution comprising 25% human serum albumin (“human serum albumin solution”). In some aspects, the method comprises combining: (a) about 1.05×10⁸ enhanced APCs, (b) about 4.99 g of a cryopreservation medium (e.g., CryoStor® CS10), (c) about 2.99 g of a hypothermic preservation medium (e.g., HypoThermasol® FRS), (d) about 2.00 g of a solution comprising 25% human serum albumin (“human serum albumin solution”).

In some aspects, the method comprises combining about 5×10⁴ to about 5×10⁹ enhanced APCs with one or more of the other components of the formulation (e.g., cryopreservation medium, hypothermic preservation medium, and/or human serum albumin). In some aspects, the method comprises adding 5×10⁵ to about 5×10⁹ enhanced APCs. In some aspects, the method comprises adding 5×10⁶ to about 5×10⁹ enhanced APCs. In some aspects, the method comprises adding 5×10⁷ to about 5×10⁹ enhanced APCs. In some aspects, the method comprises adding 5×10⁸ to about 5×10⁹ enhanced APCs. In some aspects, the method comprises adding 5×10⁴ to about 5×10⁸ enhanced APCs. In some aspects, the method comprises adding 5×10⁴ to about 5×10⁷ enhanced APCs. In some aspects, the method comprises adding 5×10⁴ to about 5×10⁶ enhanced APCs. In some aspects, the method comprises adding 5×10⁴ to about 5×10⁵ enhanced APCs. In some aspects, the method comprises adding 5×10⁵ to about 5×10⁸ enhanced APCs. In some aspects, the method comprises adding 5×10⁶ to about 5×10⁸ enhanced APCs. In some aspects, the method comprises adding 5×10⁷ to about 5×10⁸ enhanced APCs. In some aspects, the method comprises adding 5×10⁵ to about 5×10⁷ enhanced APCs. In some aspects, the method comprises adding 5×10⁶ to about 5×10⁷ enhanced APCs.

In some aspects, the method comprises adding about 1×10⁶ to about 1×10⁸ enhanced APCs. In some aspects, the method comprises adding about 1×10⁷ to about 1×10⁹ enhanced APCs. In some aspects, the method comprises adding about 1×10⁸ to about 1×10⁹ enhanced APCs. In some aspects, the method comprises adding about 1×10⁶ enhanced APCs. In some aspects, the method comprises adding about 2×10⁶ enhanced APCs. In some aspects, the method comprises adding about 3×10⁶ enhanced APCs. In some aspects, the method comprises adding about 4×10⁶ enhanced APCs. In some aspects, the method comprises adding about 5×10⁶ enhanced APCs. In some aspects, the method comprises adding about 6×10⁶ enhanced APCs. In some aspects, the method comprises adding about 7×10⁶ enhanced APCs. In some aspects, the method comprises adding about 8×10⁶ enhanced APCs. In some aspects, the method comprises adding about 9×10⁶ enhanced APCs. In some aspects, the method comprises adding about 1×10⁷ enhanced APCs. In some aspects, the method comprises adding about 2×10⁷ enhanced APCs. In some aspects, the method comprises adding about 3×10⁷ enhanced APCs. In some aspects, the method comprises adding about 4×10⁷ enhanced APCs. In some aspects, the method comprises adding about 5×10⁷ enhanced APCs. In some aspects, the method comprises adding about 6×10⁷ enhanced APCs. In some aspects, the method comprises adding about 7×10⁷ enhanced APCs. In some aspects, the method comprises adding about 8×10⁷ enhanced APCs. In some aspects, the method comprises adding about 9×10⁷ enhanced APCs. In some aspects, the method comprises adding about 1×10⁸ enhanced APCs. In some aspects, the method comprises adding about 2×10⁸ enhanced APCs. In some aspects, the method comprises adding about 3×10⁸ enhanced APCs. In some aspects, the method comprises adding about 4×10⁸ enhanced APCs. In some aspects, the method comprises adding about 5×10⁸ enhanced APCs. In some aspects, the method comprises adding about 6×10⁸ enhanced APCs. In some aspects, the method comprises adding about 7×10⁸ enhanced APCs. In some aspects, the method comprises adding about 8×10⁸ enhanced APCs. In some aspects, the method comprises adding about 9×10⁸ enhanced APCs. In some aspects, the method comprises adding comprises about 1×10⁹ enhanced APCs. In some aspects, the method comprises adding about 1.05×10⁸ enhanced APCs.

In some aspects, a method of producing a pharmaceutical formulation provided herein comprises adding a cryopreservation medium to a predetermined range. In some aspects, the cryopreservation medium is added to a concentration of about 20% to about 98% (w/w). In some aspects, the cryopreservation medium is added to a concentration of about 20% (w/w), about 25% (w/w), about 30% (w/w), about 35% (w/w), about 40% (w/w), about 45% (w/w), about 50% (w/w), about 55% (w/w), about 60% (w/w), about 65% (w/w), about 70% (w/w), about 75% (w/w), about 80% (w/w), about 85% (w/w), about 90% (w/w), about 95% (w/w), or about 98% (w/w). In some aspects, of the cryopreservation medium is added to a concentration of about 20% to about 25% (w/w), about 25% to 30% (w/w), about 30% to 35% (w/w), about 35% to 40% (w/w), about 40% to 45% (w/w), about 45% to 50% (w/w), about 50% to 55% (w/w), about 55% to 60% (w/w), about 60% to 65% (w/w), about 65% to 70% (w/w), about 70% to 75% (w/w), about 75% to 80% (w/w), about 80% to 85% (w/w), about 85% to 90% (w/w), or about 90% to 95% (w/w). In some aspects, the cryopreservation medium is added to a concentration of about 40% to about 95% (w/w). In some aspects, the cryopreservation medium is added to a concentration of about 50% (w/w).

Accordingly, in some aspects, provided herein is a method of producing a pharmaceutical formulation comprising enhanced APCs which are capable of activating T cells in a HLA-agnostic manner, the method comprises combining: (a) about 5×10⁶ enhanced APCs to about 1×10⁹ enhanced APCs and (b) a cryopreservation medium at a concentration of about 40% to about 95% (w/w). In some aspects, the method comprises combining: (a) 1.05×10⁸ enhanced APCs and (b) a cryopreservation medium at a concentration of about 50% (w/w). Non-limiting examples of suitable cryopreservation medium are provided elsewhere in the present disclosure. In some aspects, the cryopreservation medium comprises DMSO. In some aspects, the cryopreservation medium comprises CryoStor® CS10.

In some aspects, a method of producing a pharmaceutical formulation provided herein comprises adding a hypothermic preservation medium to a predetermined range. In some aspects, the hypothermic preservation medium is added to a concentration of about 10% to about 70% (w/w). In some aspects, the hypothermic preservation medium is added to a concentration of about of about 10% (w/w), about 15% (w/w), about 20% (w/w), about 25% (w/w), about 30% (w/w), about 35% (w/w), about 40% (w/w), about 45% (w/w), about 50% (w/w), about 55% (w/w), about 60% (w/w), about 65% (w/w), or about 70% (w/w). In some aspects, the hypothermic preservation medium is added to a concentration of about 10% to about 15% (w/w), about 15% to about 20% (w/w), about 20% to about 25% (w/w), about 25% to about 30% (w/w), about 30% to about 35% (w/w), about 35% to about 40% (w/w), about 40% to about 45% (w/w), about 45% to about 50% (w/w), about 50% to about 55% (w/w), about 55% to about 60% (w/w), about 60% to about 65% (w/w), or about 65% to about 70% (w/w). In some aspects, the hypothermic preservation medium is added to a concentration of about 25% to about 35% (w/w). In some aspects, the hypothermic preservative medium is added to a concentration of about 30% (w/w).

In some aspects, provided herein is a method of producing a pharmaceutical formulation comprising enhanced APCs which are capable of activating T cells in a HLA-agnostic manner, the method comprises combining: (a) about 5×10⁶ enhanced APCs to about 1×10⁹ enhanced APCs and (b) a hypothermic preservation medium at a concentration of about 25% to about 35% (w/w). In some aspects, the method comprises combining: (a) 1.05×10⁸ enhanced APCs and (b) a hypothermic preservation medium at a concentration of about 30% (w/w). In some aspects, the method comprises combining: (a) about 5×10⁶ enhanced APCs to about 1×10⁹ enhanced APCs, (b) a cryopreservation medium at a concentration of about 40% to about 95% (w/w), and (c) a hypothermic preservation medium at a concentration of about 25% to about 35% (w/w). In some aspects, the method comprises combining: (a) 1.05×10⁸ enhanced APCs, (b) a cryopreservation medium at a concentration of about 50% (w/w), and (c) a hypothermic preservation medium at a concentration of about 30% (w/w).

Non-limiting examples of suitable hypothermic preservation medium are provided elsewhere in the present disclosure. In some aspects, the hypothermic preservation medium comprises a water soluble analog of vitamin E. In some aspects, wherein the hypothermic preservation medium comprises trolox ((±)-6-hydroxy-2,5,7,8-tetramethylchromane-2-carboxylic acid). In some aspects, the hypothermic preservation medium is HypoThermasol® FRS.

In some aspects, a method of producing a pharmaceutical formulation provided herein comprises adding human serum albumin to a predetermined range. As described herein, in some aspects, the human serum albumin is provided in a solution comprising about 25% human serum albumin. In some aspects, the human serum albumin solution is added to a concentration of about 2%, about 3%, about 4%, about 5%, about 8%, about 10%, about 15%, about 20%, about 25%, about 30%, about 35%, about 40%, about 45%, or about 50% (w/w). In some aspects, the human serum albumin solution is added to a concentration of about 2% to about 3%, about 3% to about 5%, about 5% to about 8%, about 8% to about 10%, about 10% to about 15%, about 15% to about 20%, about 20% to about 25%, about 25% to about 30%, about 30% to about 35%, about 35% to about 40%, about 40% to about 45%, or about 45% to about 50% (w/w). In some aspects, the human serum albumin solution is added to a concentration of about 15% to about 25% (w/w). In some aspects, the human serum albumin solution is added to a concentration of about 20% (w/w).

In some aspects, the method comprises combining: (a) enhanced APCs and (b) a 25% human serum albumin solution at a concentration of about 15% to about 25% (w/w). In some aspects, the method comprises combining: (a) enhanced APCs, (b) a cryopreservation medium at a concentration of about 40% to about 95% (w/w), and (c) a 25% human serum albumin solution at a concentration of about 15% to about 25% (w/w). In some aspects, the method comprises combining: (a) enhanced APCs, (b) a hypothermic preservation medium at a concentration of about 25% to about 35% (w/w), and (c) a 25% human serum albumin solution at a concentration of about 15% to about 25% (w/w). In some aspects, the method comprises combining: (a) enhanced APCs, (b) a cryopreservation medium at a concentration of about 40% to about 95% (w/w), (c) a hypothermic preservation medium at a concentration of about 25% to about 35% (w/w), and (d) a 25% human serum albumin solution at a concentration of about 15% to about 25% (w/w). In some aspects, the method comprises combining: (a) enhanced APCs comprising a HPV antigen (e.g., HPV E6 and/or E7 protein), (b) a cryopreservation medium at a concentration of about 50% (w/w), (c) a hypothermic preservation medium at a concentration of about 30% (w/w), and (d) a 25% human serum albumin solution at a concentration of about 20% (w/w).

In some aspects, a method of producing a pharmaceutical formulation provided herein comprises adjusting the formulation to a desired pH. The pH can be adjusted using any suitable methods known in the art. In some aspects, the method comprises adjusting the pH of the formulation to about 5.0 to about 9.5. In some aspects, the method comprises adjusting the pH to about 6.0 to about 8.5. In some aspects, the method comprises adjusting the pH to about 7.9. In some aspects, the method comprises adjusting the pH to about 5, about 5.5, about 6, about 6.5, about 7, about 7.5, about 8, about 8.5, about 9, or about 10. In some aspects, the method comprises adjusting the pH to about 7, about 7.1, about 7.2, about 7.3, about 7.4, about 7.5, about 7.6, about 7.7, about 7.8, about 7.9, or about 8.0. In some aspects, the method comprises adjusting the pH to about 5 to about 6, about 6 to about 7, about 7 to about 8, about 8 to about 9, or about 9 to about 10. In some aspects, the method comprises adjusting the pH to about 7 to about 7.1, about 7.1 to about 7.2, about 7.2 to about 7.3, about 7.3 to about 7.4, about 7.4 to about 7.5, about 7.5 to about 7.6, about 7.6 to about 7.7, about 7.7 to about 7.8, about 7.8 to about 7.9, or about 7.9 to about 8.0.

### Methods of Producing Enhanced APCs

As described herein, enhanced APCs of the pharmaceutical formulations described herein exhibit certain improved properties compared to other APCs available in the art. For instance, the enhanced APCs described herein are capable of activating T cells in a HLA-agnostic manner. Not to be bound by any one theory, in some aspects, the APCs provided herein are able to do so because they have been modified to comprise full-length HPV antigens. Specifically, in some aspects, the enhanced APCs comprise the full-length protein of a HPV E6 protein. In some aspects, the enhanced APCs comprise the full-length protein of a HPV E7 protein. In some aspects, the enhanced APCs comprise both the full-length protein of a HPV E7 protein and the full-length protein of a HPV E6 protein.

Additionally, as further described elsewhere in the present disclosure, in some aspects, enhanced APCs useful for the present disclosure exhibits increased expression of a co-stimulatory molecule and/or a cytokine. More specifically, in some aspects, enhanced cells described herein exhibit an increased expression of IL-2 (e.g., membrane-bound IL-2). In some aspects, enhanced cells described herein exhibit an increased expression of IL-12 (e.g., membrane-bound IL-12). In some aspects, enhanced cells described herein exhibit an increased expression of CD86. In some aspects, enhanced cells described herein exhibit an increased expression of IL-2 (e.g., membrane-bound IL-2), IL-12 (e.g., membrane-bound IL-12), and CD86. Therefore, in some aspects, enhanced cells provided herein comprises both the full-length protein of a HPV E7 protein (e.g., of HPV-16) and the full-length protein of a HPV E6 protein (e.g., of HPV-16), and exhibits increased expression of IL-2 (e.g., membrane-bound IL-2), IL-12 (e.g., membrane-bound IL-12), and CD86.

In some aspects, modifying the APCs to produce the enhanced APCs useful for the formulations provided herein comprises intracellularly delivering a nucleic acid encoding an antigen (e.g., HPV antigen), co-stimulatory molecule (e.g., CD86), and/or cytokine (e.g., membrane-bound IL-2 and/or membrane-bound IL-12). In some aspects, intracellularly delivering a nucleic acid to a cell comprises passing a cell suspension comprising the cell through a constriction under a set of parameters, thereby causing a perturbation within the cell such that the nucleic acid enters the cell through the perturbation when contacted with the cell (i.e., squeeze processing). In some aspects, the method further comprises contacting the cell with the nucleic acid. As used herein, “contacting” between a cell and a nucleic acid described herein does not require that the cell and the nucleic acid be in physical contact. As is apparent from the present disclosure, contacting between a cell and a nucleic acid occurs as long as the nucleic acid is capable of entering the cell once there are perturbations within the cell membrane of the cell. To help illustrate, in some aspects, a cell and a nucleic acid are in contact if they are both present within the same cell suspension, regardless of whether the cell and the nucleic acid are in physical contact or not. Accordingly, in some aspects, contacting the cell with the nucleic acid comprises incubating the cell suspension comprising the cell with the nucleic acid.

In some aspects, where multiple nucleic acids are being delivered (e.g., a nucleic acid encoding a HPV antigen, a nucleic acid encoding a co-stimulatory molecule, and a nucleic acid encoding a cytokine), they can be delivered to a cell using a single squeeze processing e.g., a cell suspension comprises the multiple nucleic acids, which are delivered to the cell in combination; “concurrent delivery”). In some aspects, the multiple nucleic acids can be delivered to a cell sequentially. As used herein, the term “sequential delivery” refers to the delivery of multiple nucleic acids to a cell, where a first nucleic acid (e.g., encoding a HPV antigen) is delivered to the cell and then the second (or subsequent) nucleic acid (e.g., encoding a co-stimulatory molecule and/or cytokine) is delivered to the cell. In some aspects, the first nucleic acid, the second nucleic acid, or both the first and second nucleic acids can be delivered to the cell using squeeze processing. For instance, in some aspects, the first nucleic acid can be delivered to the cell using squeeze processing, and the second nucleic acid can be delivered to the cell using non-squeeze processing (e.g., transfection). In some aspects, the first nucleic acid can be delivered to the cell using non-squeeze processing (e.g., transfection), and the second nucleic acid can be delivered to the cell using squeeze processing. In some aspects, the first nucleic acid can be delivered to the cell using a first squeeze, and then the second nucleic acid can be delivered to the cell using a second squeeze (also referred to herein as “sequential squeeze” or “sequential squeeze processing”). Accordingly, sequential delivery useful for the present disclosure can comprise multiple squeeze processing. In some aspects, each of the multiple squeeze processing delivers a separate nucleic acid to the cell. In some aspects, one or more of the multiple squeeze processing do not involve the delivery of a nucleic acid. For instance, in some aspects, a sequential delivery method described herein comprises a first squeeze, a second squeeze, and a third squeeze, wherein the first squeeze comprises passing a cell without any payload through a first constriction, the second squeeze comprises passing the cell from the first squeeze through a second constriction to deliver a first nucleic acid (e.g., encoding a HPV antigen) to the cell, and the third squeeze comprises passing the cell from the second squeeze through a third constriction to deliver a second nucleic acid (e.g., encoding a co-stimulatory molecule and/or cytokine) to the cell. Not to be bound by any one theory, in some aspects, passing the cell through the first constriction without any payload (i.e., the first squeeze) can help prepare the cell for subsequent deliveries, e.g., can improve the delivery efficiency of the first nucleic acid and/or the second nucleic acid.

### Constrictions

### Microfluidic Channels

As described herein, a constriction is used to cause a physical deformity in the cells, such that perturbations are created within the cell membrane of the cells, allowing for the delivery of a nucleic acid into the cell. In some aspects, a constriction is within a channel contained within a microfluidic device (referred to herein as “microfluidic channel” or “channel”). Where multiple channels are involved, in some aspects, the multiple channels can be placed in parallel and/or in series within the microfluidic device. In some aspects, the cells described herein can be passed through at least about 2, at least about 3, at least about 4, at least about 5, at least about 6, at least about 7, at least about 8, at least about 9, at least about 10, at least about 20, at least about 30, at least about 40, at least about 50, at least about 75, at least about 100, at least about 150, at least about 200, at least about 250, at least about 300, at least about 350, at least about 400, at least about 450, at least about 500, at least about 550, at least about 600, at least about 650, at least about 700, at least about 750, at least about 800, at least about 850, at least about 900, at least about 950, at least about 1,000 or more separate constrictions. In some aspects, the cells described herein are passed through more than about 1,000 separate constrictions. In some aspects, the multiple constrictions can be part of a single microfluidic device (e.g., multi-row constriction chip). In some aspects, one or more of the multiple constrictions can be part of different microfluidic devices. For instance, in some aspects, the cells described herein undergo a first squeeze processing, in which the cells pass through a first constriction in a first microfluidic device (e.g., chip). Then, after the cells have undergone the first squeeze processing (e.g., passed through the first constriction), the cells undergo a second squeeze processing, in which the cells pass through a second constriction in a second microfluidic device (e.g., chip). In some aspects, each of the constrictions are the same (e.g., has the same length, width, and/or depth). In some aspects, one or more of the constrictions are different. Where plurality of constrictions are used, the plurality of constrictions can comprise a first constriction which is associated with a first nucleic acid (e.g., encoding a HPV antigen), and a second constriction which is associated with a second nucleic acid (e.g., encoding a co-stimulatory molecule and/or cytokine), wherein the cell suspension passes through the first constriction such that the first nucleic acid is delivered to one or more cells of the plurality of cells, and then the cell suspension passes through the second constriction such that the second nucleic acid is delivered to the one or more cells of the plurality of cells. In some aspects, the cell suspension is passed through the second constriction at least about 1 minute, at least about 30 minutes, at least about 1 hour, at least about 6 hours, at least about 12 hours, or at least about 1 day after the cell suspension is passed through the first constriction.

In some aspects, where the cell suspension is passed through multiple constrictions (e.g., multiple squeeze processing), the cells remain viable after passing through each constriction. As is apparent from the present disclosure, in some aspects, multiple constrictions can comprise two or more constrictions present within a single microfluidic device (e.g., multi-row constriction chip), such the cells pass through the multiple constrictions sequentially. In some aspects, the multiple constrictions are part of separate microfluidic devices, such that a first constriction is associated with a first microfluidic device and a second constriction is associated with a second microfluidic device. For instance, in some aspects, cells are passed through a first constriction (i.e., first squeeze processing), which is associated with a first microfluidic device (e.g., chip). After the cells have passed through the first constriction, the cells are passed through a second constriction (i.e., second squeeze processing), which is associated with a second microfluidic device (e.g., chip). In some aspects, after passing through the first constriction, the cells are cultured in a medium prior to passing the cells through the second constriction. In some aspects, the cells are cultured for at least about 1 minute, at least about 30 minutes, at least about 1 hour, at least about 6 hours, at least about 12 hours, or at least about 1 day before passing the cells through the second constriction. As is apparent from the present disclosure, in some aspects, the first and second constrictions have the same length, depth, and/or width. In some aspects, the first and second constrictions can have different length, depth, and/or width.

In some aspects, after passing through a constriction, at least about 50%, at least about 55%, at least about 60%, at least about 65%, at least about 70%, at least about 75%, at least about 80%, at least about 85%, at least about 90%, at least about 95%, or about 100% of the cells remain viable. Where the cells pass through multiple constrictions (e.g., part of a single microfluidic device or separate microfluidic devices), at least about 50%, at least about 55%, at least about 60%, at least about 65%, at least about 70%, at least about 75%, at least about 80%, at least about 85%, at least about 90%, at least about 95%, or about 100% of the cells remain viable after passing through each of the multiple constrictions. The viability of the cells can be measured using any suitable methods known in the art. In some aspects, the viability of the cells can be measured using a Nucleocounter NC-200, an Orflo Moxi Go II Cell Counter, or both.

Exemplary microfluidic channels containing cell-deforming constrictions for use in the methods disclosed herein are described in US Publ. No. 2020/0277566 A1, US Publ. No. 2020/0332243 A1, US Publ. No. 2020/0316604 A1, U.S. Provisional Appl. No. 63/131,423, and U.S. Provisional Appl. No. 63/131,430, each of which is incorporated herein by reference in its entirety.

In some aspects, a microfluidic channel described herein (i.e., comprising a constriction) includes a lumen and is configured such that a cell suspended in a buffer (e.g., cell suspension) can pass through the channel. Microfluidic channels useful for the present disclosure can be made using any suitable materials available in the art, including, but not limited to, silicon, metal (e.g., stainless steel), plastic (e.g., polystyrene), ceramics, glass, crystalline substrates, amorphous substrates, polymers (e.g., Poly-methyl methacrylate (PMMA), PDMS, Cyclic Olefin Copolymer (COC)), or combinations thereof. In some aspects, the material is silicon. Fabrication of the microfluidic channel can be performed by any method known in the art, including, but not limited to, dry etching for example deep reactive ion etching, wet etching, photolithography, injection molding, laser ablation, SU-8 masks, or combinations thereof. In some aspects, the fabrication is performed using dry etching.

In some aspects, a microfluidic channel useful for the present disclosure comprises an entrance portion, a center point, and an exit portion. In some aspects, the cross-section of one or more of the entrance portion, the center point, and/or the exit portion can vary. For example, the cross-section can be circular, elliptical, an elongated slit, square, hexagonal, or triangular in shape.

The entrance portion defines a constriction angle. In some aspects, by modulating (e.g., increasing or decreasing) the constriction angle, any clogging of the constriction can be reduced or prevented. In some aspects, the angle of the exit portion can also be modulated. For example, in some aspects, the angle of the exit portion can be configured to reduce the likelihood of turbulence that can result in non-laminar flow. In some aspects, the walls of the entrance portion and/or the exit portion are linear. In some aspects, the walls of the entrance portion and/or the exit portion are curved.

In some aspects, the length, depth, and/or width of the constriction can vary. In some aspects, by modulating (e.g., increasing or decreasing) the length, depth, and/or width of the constriction, the delivery efficiency of a payload can be regulated. As used herein, the term “delivery efficiency” refers to the amount of payload that is delivered into the cell. For instance, an increased delivery efficiency can occur when the total amount of payload that is delivered is increased.

In some aspects, the constriction has a length of less than about 1 m. In some aspects the constriction has a length of about 0 μm to about 100 μm. In some aspects, the length of the constriction is less than about 0.1 μm, less than about 0.2 μm, less than about 0.3 μm, less than about 0.4 μm, less than about 0.5 μm, less than about 0.6 μm, less than about 0.7 μm, less than about 0.8 μm, less than about 0.9 μm, less than about 1 μm, less than about 2.5 μm, less than about 5 μm, less than about 7.5 μm, less than about 10 μm, less than about 12.5 μm, less than about 15 μm, less than about 20 μm, less than about 30 μm, less than about 40 μm, less than about 50 μm, less than about 60 μm, less than about 70 μm, less than about 80 μm, less than about 90 μm, or less than about 100 μm. In some aspects, the length of the constriction is about 0.1 μm, about 0.2 μm, about 0.3 μm, about 0.4 μm, about 0.5 μm, about 0.6 μm, about 0.7 μm, about 0.8 μm, about 0.9 μm, about 1 μm, about 2.5 μm, about 5 μm, about 7.5 μm, about 10 μm, about 12.5 μm, about 15 μm, about 20 μm, about 30 μm, about 40 μm, about 50 μm, about 60 μm, about 70 μm, about 80 μm, about 90 μm, or about 100 μm. In some aspects, the length of the constriction is about 10 μm. In some aspects, the constriction has a length of about 0 μm. For example, in some aspects, a microfluidic device (e.g., chip) useful for the present disclosure comprises a constriction that resembles two points of a diamond coming together, such that the length of the constriction is about 0 μm.

In some aspects, the width of the constriction is between about 0 μm to about 10 μm. In some aspects, the width of the constriction is less than about 0.1 μm, less than about 0.2 μm, less than about 0.3 μm, less than about 0.4 μm, less than about 0.5 μm, less than about 0.6 μm, less than about 0.7 μm, less than about 0.8 μm, less than about 0.9 μm, less than about 1 μm, less than about 2 μm, less than about 3 μm, less than about 4 μm, less than about 5 μm, less than about 6 μm, less than about 7 μm, less than about 8 μm, less than about 9 μm, or less than about 10 μm.

In some aspects, the width of the constriction is about 0.1 μm, about 0.2 μm, about 0.3 μm, about 0.4 μm, about 0.5 μm, about 0.6 μm, about 0.7 μm, about 0.8 μm, about 0.9 μm, about 1 μm, about 2 μm, about 3 μm, about 4 μm, about 5 μm, about 6 μm, about 7 μm, about 8 μm, about 9 μm, or about 10 μm. In some aspects, width of the constriction is between about 3 μm to about 10 μm. In some aspects, the width of the constriction is about 6 μm.

In some aspects, the depth of the constriction is at least about 1 μm. In some aspects, the depth of the constriction is at least about 2 μm, at least about 3 μm, at least about 4 μm, at least about 5 μm, at least about 10 μm, at least about 20 μm, at least about 30 μm, at least about 40 μm, at least about 50 μm, at least about 60 μm, at least about 70 μm, at least about 80 μm, at least about 90 μm, at least about 100 μm, at least about 110 μm, or at least about 120 μm. In some aspects, the depth of the constriction is about 5 μm to about 90 μm. In some aspects, the depth of the constriction is about 5 μm, about 10 μm, about 20 μm, about 30 μm, about 40 μm, about 50 μm, about 60 μm, about 70 μm, about 80 μm, or about 90 μm. In some aspects, the depth of the constriction is about 70 μm.

In some aspects, the length of the constriction is about 10 μm, the width of the constriction is about 6 μm, and the depth of the constriction is about 70 μm. In some aspects, the length of the constriction is 10 μm, the width of the constriction is 6 μm, and the depth of the constriction is 70 μm.

In some aspects, the diameter of a constriction (e.g., contained within a microfluidic channel) is a function of the diameter of one or more cells that are passed through the constriction. Not to be bound by any one theory, in some aspects, the diameter of the constriction is less than that of the cells, such that a deforming force is applied to the cells as they pass through the constriction, resulting in the transient physical deformity of the cells.

Accordingly, in some aspects, the diameter of the constriction (also referred to herein as “constriction size”) is about 20% to about 99% of the diameter of the cell. In some aspects, the constriction size is about 20%, about 30%, about 40%, about 50%, about 60%, about 70%, about 80%, about 90%, or about 99% of the cell diameter. As is apparent from the present disclosure, by modulating (e.g., increasing or decreasing) the diameter of a constriction, the delivery efficiency of a payload into a cell can also be regulated.

### Surface Having Pores

In some aspects, a constriction described herein comprises a pore, which is contained in a surface. Non-limiting examples of pores contained in a surface that can be used with the present disclosure are described in, e.g., US Publ. No. 2019/0382796 A1, which is incorporated herein by reference in its entirety.

In some aspects, a surface useful for the present disclosure (i.e., comprising one or more pores that can cause a physical deformity in a cell as it passes through the pore) can be made using any suitable materials available in the art and/or take any one of a number of forms. Non-limiting examples of such materials include synthetic or natural polymers, polycarbonate, silicon, glass, metal, alloy, cellulose nitrate, silver, cellulose acetate, nylon, polyester, polyethersulfone, polyacrylonitrile (PAN), polypropylene, PVDF, polytetrafluorethylene, mixed cellulose ester, porcelain, ceramic, or combinations thereof.

In some aspects, the surface comprises a filter. In some aspects, the filter is a tangential flow filter. In some aspects, the surface comprises a membrane. In some aspects, the surface comprises a sponge or sponge-like matrix. In some aspects, the surface comprises a matrix. In some aspects, the surface comprises a tortuous path surface. In some aspects, the tortuous path surface comprises cellulose acetate.

The surface disclosed herein (i.e., comprising one or more pores) can have any suitable shape known in the art. Where the surface has a 2-dimensional shape, the surface can be, without limitation, circular, elliptical, round, square, star-shaped, triangular, polygonal, pentagonal, hexagonal, heptagonal, or octagonal. In some aspects, the surface is round in shape. Where the surface has a 3-dimensional shape, in some aspects, the surface can be, without limitation, cylindrical, conical, or cuboidal.

As is apparent from the present disclosure, a surface that is useful for the present disclosure (e.g., comprising one or more pores) can have various cross-sectional widths and thicknesses. In some aspects, the cross-sectional width of the surface is between about 1 mm and about 1 m. In some aspects, the surface has a defined thickness. In some aspects, the surface thickness is uniform. In some aspects, the surface thickness is variable. For example, in some aspects, certain portions of the surface are thicker or thinner than other portions of the surface. In such aspects, the thickness of the different portions of the surface can vary by about 1% to about 90%. In some aspects, the surface is between about 0.01 m to about 5 mm in thickness.

The cross-sectional width of the pores can depend on the type of cell that is being targeted with a payload. In some aspects, the pore size is a function of the diameter of the cell of cluster of cells to be targeted. In some aspects, the pore size is such that a cell is perturbed (i.e., physically deformed) upon passing through the pore. In some aspects, the pore size is less than the diameter of the cell. In some aspects, the pore size is about 20% to about 99% of the diameter of the cell. In some aspects, the pore size is about 20%, about 30%, about 40%, about 50%, about 60%, about 70%, about 80%, about 90%, or about 99% of the diameter of the cell. In some aspects, the pore size is about 0.4 μm, about 0.5 μm, about 0.6 μm, about 0.7 μm, about 0.8 μm, about 0.9 μm, about 1 μm, about 2 μm, about 3 μm, about 4 μm, about 5 μm, about 6 μm, about 7 am, about am, about 9 μm, about 10 μm, about 11 μm, about 12 μm, about 13 μm, about 14 μm, or about 15 μm or more.

The entrances and exits of a pore can have a variety of angles. In some aspects, by modulating (e.g., increasing or decreasing) the pore angle, any clogging of the pore can be reduced or prevented. In some aspects, the flow rate (i.e., the rate at which a cell or a suspension comprising the cell passes through the pore) is between about 0.001 mL/cm/see to about 100 L/cm/sec. For example, the angle of the entrance or exit portion can be between about 0 and about 90 degrees. In some aspects, the pores have identical entrance and exit angles. In some aspects, the pores have different entrance and exit angles. In some aspects, the pore edge is smooth, e.g., rounded or curved. As used herein, a “smooth” pore edge has a continuous, flat, and even surface without bumps, ridges, or uneven parts. In some aspects, the pore edge is sharp.

As used herein, a “sharp” pore edge has a thin edge that is pointed or at an acute angle. In some aspects, the pore passage is straight. As used herein, a “straight” pore passage does not contain curves, bends, angles, or other irregularities. In some aspects, the pore passage is curved. As used herein, a “curved” pore passage is bent or deviates from a straight line. In some aspects, the pore passage has multiple curves, e.g., about 2, about 3, about 4, about 5, about 6, about 7, about 8, about 9, about 10 or more curves.

The pores can have any shape known in the art, including a 2-dimensional or 3-dimensional shape. The pore shape (e.g., the cross-sectional shape) can be, without limitation, circular, elliptical, round, square, star-shaped, triangular, polygonal, pentagonal, hexagonal, heptagonal, and octagonal. In some aspects, the cross-section of the pore is round in shape. In some aspects, the 3-dimensional shape of the pore is cylindrical or conical. In some aspects, the pore has a fluted entrance and exit shape. In some aspects, the pore shape is homogenous (i.e., consistent or regular) among pores within a given surface. In some aspects, the pore shape is heterogeneous (i.e., mixed or varied) among pores within a given surface.

A surface useful for the present disclosure can have a single pore. In some aspects, a surface useful for the present disclosure comprises multiple pores. In some aspects, the pores encompass about 10% to about 80% of the total surface area of the surface. In some aspects, the surface contains about 1.0×10⁵ to about 1.0×10³⁰ total pores. In some aspects, the surface comprises between about 10 and about 1.0×10¹⁵ pores per mm² surface area.

The pores can be distributed in numerous ways within a given surface. In some aspects, the pores are distributed in parallel within a given surface. In some aspects, the pores are distributed side-by-side in the same direction and are the same distance apart within a given surface. In some aspects, the distribution of the pores is ordered or homogeneous. In such aspects, the pores can be distributed in a regular, systematic pattern, or can be the same distance apart within a given surface. In some aspects, the distribution of the pores is random or heterogeneous. For instance, in some aspects, the pores are distributed in an irregular, disordered pattern, or are different distances apart within a given surface.

In some aspects, multiple surfaces are used, such that a cell passes through multiple pores, wherein the pores are on different surfaces. In some aspects, multiple surfaces are distributed in series. The multiple surfaces can be homogeneous or heterogeneous in surface size, shape, and/or roughness. The multiple surfaces can further contain pores with homogeneous or heterogeneous pore size, shape, and/or number, thereby enabling the simultaneous delivery of a range of payloads into different cell types.

In some aspects, an individual pore, e.g., of a surface that can be used with the present disclosure, has a uniform width dimension (i.e., constant width along the length of the pore passage). In some aspects, an individual pore has a variable width (i.e., increasing or decreasing width along the length of the pore passage). In some aspects, pores within a given surface have the same individual pore depths. In some aspects, pores within a given surface have different individual pore depths. In some aspects, the pores are immediately adjacent to each other. In some aspects, the pores are separated from each other by a distance. In some aspects, the pores are separated from each other by a distance of about 0.001 m to about 30 mm.

In some aspects, the surface is coated with a material. The material can be selected from any material known in the art, including, without limitation, Teflon, an adhesive coating, surfactants, proteins, adhesion molecules, antibodies, anticoagulants, factors that modulate cellular function, nucleic acids, lipids, carbohydrates, transmembrane proteins, or combinations thereof. In some aspects, the surface is coated with polyvinylpyrrolidone. In some aspects, the material is covalently attached to the surface. In some aspects, the material is non-covalently attached to the surface. In some aspects, the surface molecules are released at the cells pass through the pores.

In some aspects, the surface has modified chemical properties. In some aspects, the surface is hydrophilic. In some aspects, the surface is hydrophobic. In some aspects, the surface is charged. In some aspects, the surface is positively and/or negatively charged. In some aspects, the surface can be positively charged in some regions and negatively charged in other regions. In some aspects, the surface has an overall positive or overall negative charge. In some aspects, the surface can be any one of smooth, electropolished, rough, or plasma treated. In some aspects, the surface comprises a zwitterion or dipolar compound. In some aspects, the surface is plasma treated.

In some aspects, the surface is contained within a larger module. In some aspects, the surface is contained within a syringe, such as a plastic or glass syringe. In some aspects, the surface is contained within a plastic filter holder. In some aspects, the surface is contained within a pipette tip.

### Cell Perturbation

As described herein, as a cell passes through a constriction, it becomes physically deformed, such that there is a perturbation (e.g., a hole, tear, cavity, aperture, pore, break, gap, perforation) in the cell membrane of the cell. Such perturbation in the cell membrane is temporary and sufficient for any of the nucleic acids described herein to be delivered into the cell. Cells have self-repair mechanisms that allow the cells to repair any disruption in their cell membrane. See Blazek et al., Physiology (Bethesda) 30(6): 438-48 (November 2015), which is incorporated herein by reference in its entirety. Accordingly, in some aspects, once the cells have passed through the constriction (e.g., microfluidic channel or pores), the perturbations in the cell membrane can be reduced or eliminated, such that the payload that was delivered into the cell does not exit the cell.

In some aspects, the perturbation in the cell membrane lasts from about 1.0×10⁻⁹ seconds to about 2 hours after the pressure is removed (e.g., cells have passed through the constriction). In some aspects, the cell perturbation lasts for about 1.0×10⁻⁹ second to about 1 second, for about 1 second to about 1 minute, or for about 1 minute to about 1 hour. In some aspects, the cell perturbation lasts for between about 1.0×10⁻⁹ second to about 1.0×10⁻¹ second, between about 1.0×10⁻⁹ second to about 1.0×10⁻² second, between about 1.0×10⁻⁹ second to about 1.0×10⁻³ second, between about 1.0×10⁻⁹ second to about 1.0×10⁻⁴ second, between about 1.0×10⁻⁹ second to about 1.0×10⁻⁵ second, between about 1.0×10⁻⁹ second to about 1.0×10⁻⁶ second, between about 1.0×10⁻⁹ second to about 1.0×10⁻⁷ second, or between about 1.0×10⁻⁹ second to about 1.0×10⁻⁸ second. In some aspects, the cell perturbation lasts for about 1.0×10⁻⁸ second to about 1.0×10⁻¹ second, for about 1.0×10⁻⁷ second to about 1.0×10⁻¹ second, about 1.0×10⁻⁶ second to about 1.0×10⁻¹ second, about 1.0×10⁻⁵ second to about 1.0×10⁻¹ second, about 1.0×10⁻⁴ second to about 1.0×10⁻¹ second, about 1.0×10⁻³ second to about 1.0×10⁻¹ second, or about 1.0×10⁻² second to about 1.0×10⁻¹ second. The cell perturbations (e.g., pores or holes) created by the methods described herein are not formed as a result of assembly of polypeptide subunits to form a multimeric pore structure such as that created by complement or bacterial hemolysins.

In some aspects, as the cell passes through the constriction, the pressure applied to the cells temporarily imparts injury to the cell membrane that causes passive diffusion of material through the perturbation. In some aspects, the cell is only deformed or perturbed for a brief period of time, e.g., on the order of 100 s or less to minimize the chance of activating apoptotic pathways through cell signaling mechanisms, although other durations are possible (e.g., ranging from nanoseconds to hours). In some aspects, the cell is deformed for less than about 1.0×10⁻⁹ second to less than about 2 hours. In some aspects, the cell is deformed for less than about 1.0×10⁻⁹ second to less than about 1 second, less than about 1 second to less than about 1 minute, or less than about 1 minute to less than about 1 hour. In some aspects, the cell is deformed for about 1.0×10⁻⁹ second to about 2 hours. In some aspects, the cell is deformed for about 1.0×10⁻⁹ second to about 1 second, about 1 second to about 1 minute, or about 1 minute to about 1 hour. In some aspects, the cell is deformed for between any one of about 1.0×10⁻⁹ second to about 1.0×10⁻¹ second, about 1.0×10⁻⁹ second to about 1.0×10⁻² second, about 1.0×10⁻⁹ second to about 1.0×10⁻³ second, about 1.0×10⁻⁹ second to about 1.0×10⁻⁴ second, about 1.0×10⁻⁹ second to about 1.0×10⁻⁵ second, about 1.0×10⁻⁹ second to about 1.0×10⁻⁶ second, about 1.0×10⁻⁹ second to about 1.0×10⁻⁷ second, or about 1.0×10⁻⁹ second to about 1.0×10⁻¹ second. In some aspects, the cell is deformed or perturbed for about 1.0×10⁻¹ second to about 1.0×10⁻¹ second, for about 1.0×10⁻⁷ second to about 1.0×10⁻¹ second, about 1.0×10⁻⁶ second to about 1.0×10⁻¹ second, about 1.0×10⁻⁵ second to about 1.0×10⁻¹ second, about 1.0×10⁻⁴ second to about 1.0×10⁻¹ second, about 1.0×10⁻³ second to about 1.0×10⁻¹ second, or about 1.0×10⁻² second to about 1.0×10⁻¹ second. In some aspects, deforming the cell includes deforming the cell for a time ranging from, without limitation, about 1 μs to at least about 750 μs, e.g., at least about 1 μs, at least about 10 μs, at least about 50 μs, at least about 100 μs, at least about 500 μs, or at least about 750 μs.

In some aspects, the delivery of a nucleic acid described herein into the cell occurs simultaneously with the cell passing through the constriction. In some aspects, delivery of the nucleic acid into the cell can occur after the cell passes through the constriction (i.e., when perturbation of the cell membrane is still present and prior to cell membrane of the cells being restored). In some aspects, delivery of the nucleic acid into the cell occurs on the order of minutes after the cell passes through the constriction. In some aspects, a perturbation in the cell after it passes through the constriction is corrected within the order of about five minutes after the cell passes through the constriction.

In some aspects, the viability of a cell after passing through a constriction is about 5% to about 100%. In some aspects, the cell viability after passing through the constriction is at least about 5%, at least about 10%, at least about 20%, at least about 30%, at least about 40%, at least about 50%, at least about 60%, at least about 70%, at least about 75%, at least about 80%, at least about 85%, at least about 90%, at least about 95%, or at least about 99%. In some aspects, the cell viability is measured from about 1.0×10⁻² second to at least about 10 days after the cell passes through the constriction. For example, the cell viability can be measured from about 1.0×10⁻² second to about 1 second, about 1 second to about 1 minute, about 1 minute to about 30 minutes, or about 30 minutes to about 2 hours after the cell passes through the constriction. In some aspects, the cell viability is measured about 1.0×10⁻² second to about 2 hours, about 1.0×10⁻² second to about 1 hour, about 1.0×10⁻² second to about 30 minutes, about 11.0×10⁻² second to about 1 minute, about 1.0×10⁻² second to about 30 seconds, about 1.0×10⁻² second to about 1 second, or about 1.0×10⁻² second to about 0.1 second after the cell passes through the constriction. In some aspects, the cell viability is measured about 1.5 hours to about 2 hours, about 1 hour to about 2 hours, about 30 minutes to about 2 hours, about 15 minutes to about 2 hours, about 1 minute to about 2 hours, about 30 seconds to about 2 hours, or about 1 second to about 2 hours after the cell passes through the constriction. In some aspects, the cell viability is measured about 2 hours to about 5 hours, about 5 hours to about 12 hours, about 12 hours to about 24 hours, or about 24 hours to about 10 days after the cell passes through the constriction.

### Delivery Parameters

As is apparent from the present disclosure, a number of parameters can influence the delivery efficiency of a nucleic acid into a cell using the squeeze processing methods provided herein. Accordingly, by modulating (e.g., increasing or decreasing) one or more of the delivery parameters, the delivery of a payload into a cell can be improved. Therefore, in some aspects, the present disclosure relates to a method of increasing the delivery of a payload (e.g., nucleic acid described herein) into a cell, wherein the method comprises modulating one or more parameters under which a cell suspension is passed through a constriction, wherein the cell suspension comprises a population of the cells, and wherein the one or more parameters increase the delivery of a payload into one or more cells of the population of cells compared to a reference parameter. As described elsewhere in the present disclosure, the payload can be in contact with the population of cells before, during, or after the squeezing step.

In some aspects, by modulating one or more of the delivery parameters, the delivery of the payload (e.g., nucleic acid described herein) into the one or more cells is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 40-fold, or at least about 50-fold, compared to a delivery of the payload agent into a corresponding cell using the reference parameter.

In some aspects, the one or more delivery parameters that can be modulated to increase the delivery efficiency of a parameter comprises a cell density (i.e., the concentration of the cells present, e.g., in the cell suspension), pressure, or both. Additional examples of delivery parameters that can be modulated are provided elsewhere in the present disclosure.

In some aspects, the cell density is about 1×10⁸ cells/mL, about 2×10⁷ cells/mL, about 3×10⁷ cells/mL, about 4×10⁷ cells/mL, about 5×10⁷ cells/mL, about 6×10⁷ cells/mL, about 7×10⁷ cells/mL, about 8×10⁷ cells/mL, about 9×10⁷ cells/mL, about 1×10⁸ cells/mL, about 1.1×10⁸ cells/mL, about 1.2×10⁸ cells/mL, about 1.3×10⁸ cells/mL, about 1.4×10⁸ cells/mL, about 1.5×10⁸ cells/mL, about 2.0×10⁸ cells/mL, about 3.0×10⁸ cells/mL, about 4.0×10′ cells/mL, about 5.0×10⁸ cells/mL, about 6.0×10⁸ cells/mL, about 7.0×10⁸ cells/mL, about 8.0×10⁸ cells/mL, about 9.0×10⁸ cells/mL, or about 1.0×10⁹ cells/mL or more. In some aspects, the cell density is between about 6×10⁷ cells/mL and about 1.2×10⁸ cells/mL.

In some aspects, the pressure is about 20 psi, about 25 psi, about 30 psi, about 35 psi, about 40 psi, about 50 psi, about 55 psi, about 60 psi, about 65 psi, about 70 psi, about 75 psi, about 80 psi, about 85 psi, about 90 psi, about 95 psi, about 100 psi, about 110 psi, about 120 psi, about 130 psi, about 140 psi, about 150 psi, about 160 psi, about 170 psi, about 180 psi, about 190 psi, or about 200 psi or more. In some aspects, the pressure is between about 30 psi and about 90 psi.

In some aspects, the particular type of device (e.g., microfluidic chip) can also have an effect on the delivery efficiency of a payload described herein (e.g., nucleic acid described herein). In the case of a microfluidic chip, different chips can have different constriction parameters, e.g., length, depth, and width of the constriction; entrance angle, exit angle, length, depth, and width of the approach region, etc. As described herein, such variables can influence the delivery of a payload into a cell using the squeeze processing methods of the present disclosure. In some aspects, the length of the constriction is up to 100 μm. For instance, in some aspects, the length is about 1 μm, about 5 μm, 10 μm, about 20 μm, about 30 μm, about 40 μm, about 50 μm, about 60 μm, about 70 μm, about 80 μm, about 90 μm, or about 100 μm. In some aspects, the length of the constriction is less than 1 μm. In some aspects, the length of the constriction is less than about 1 μm, less than about 5 μm, less than about 10 μm, less than about 20 μm, less than about 30 μm, less than about 40 μm, less than about 50 μm, less than about 60 μm, less than about 70 μm, less than about 80 μm, less than about 90 μm, or less than about 100 μm. In some aspects, the constriction has a length of about 10 μm.

In some aspects, the width of the constriction is up to about 10 μm. In some aspects, the width of the constriction is less than about 1 μm, less than about 2 μm, less than about 3 μm, less than about 4 μm, less than about 5 μm, less than about 6 μm, less than about 7 μm, less than about 8 μm, less than about 9 μm, or less than about 10 μm. In some aspects, the width is between about 3 μm to about 10 μm. In some aspects, the width is about 3 μm, about 4 μm, about 5 μm, about 6 μm, about 7 μm, about 8 μm, about 9 μm, or about 10 μm. In some aspects, the width of the constriction is about 6 μm.

In some aspects, the depth of the constriction is at least about 1 μm. In some aspects, the depth of the constriction is at least about 1 μm, at least about 2 μm, at least about 3 μm, at least about 4 μm, at least about 5 μm, at least about 10 μm, at least about 20 μm, at least about 30 μm, at least about 40 μm, at least about 50 μm, at least about 60 μm, at least about 70 μm, at least about 80 μm, at least about 90 μm, at least about 100 μm, at least about 110 μm, or at least about 120 μm. In some aspects, the depth is between about 5 μm to about 90 μm. In some aspects, the depth is about 5 μm, about 10 μm, about 15 μm, about 20 μm, about 30 μm, about 40 μm, about 50 μm, about 60 μm, about 70 μm, about 80 μm, or about 90 μm. In some aspects, the depth of the constriction is about 70 μm.

In some aspects, the length is about 10 μm, the width is about 6 μm, and depth is about 70 μm.

Additional examples of parameters that can influence the delivery of a payload into the cell include, but are not limited to, the dimensions of the constriction (e.g., length, width, and/or depth), the entrance angle of the constriction, the surface properties of the constrictions (e.g., roughness, chemical modification, hydrophilic, hydrophobic), the operating flow speeds, payload concentration, the amount of time that the cell recovers, or combinations thereof. Further parameters that can influence the delivery efficiency of a payload (e.g., nucleic acid described herein) can include the velocity of the cell in the constriction, the shear rate in the constriction, the viscosity of the cell suspension, the velocity component that is perpendicular to flow velocity, and time in the constriction. Such parameters can be designed to control delivery of the payload.

In some aspects, the temperature used in the methods of the present disclosure can also have an effect on the delivery efficiency of the payloads into the cell, as well as the viability of the cell. In some aspects, the squeeze processing method is performed between about −5° C. and about 45° C. For example, the methods can be carried out at room temperature (e.g., about 20° C.), physiological temperature (e.g., about 37° C.), higher than physiological temperature (e.g., greater than about 37° C. to 45° C. or more), or reduced temperature (e.g., about −5° C. to about 4° C.), or temperatures between these exemplary temperatures.

Various methods can be utilized to drive the cells through the constrictions. For example, pressure can be applied by a pump on the entrance side (e.g., gas cylinder, or compressor), a vacuum can be applied by a vacuum pump on the exit side, capillary action can be applied through a tube, and/or the system can be gravity fed. Displacement based flow systems can also be used (e.g., syringe pump, peristaltic pump, manual syringe or pipette, pistons, etc.). In some aspects, the cells are passed through the constrictions by positive pressure. In some aspects, the cells are passed through the constrictions by constant pressure or variable pressure. In some aspects, pressure is applied using a syringe. In some aspects, pressure is applied using a pump. In some aspects, the pump is a peristaltic pump or a diaphragm pump. In some aspects, pressure is applied using a vacuum. In some aspects, the cells are passed through the constrictions by g-force. In some aspects, the cells are passed through the constrictions by capillary pressure.

In some aspects, fluid flow directs the cells through the constrictions. In some aspects, the fluid flow is turbulent flow prior to the cells passing through the constriction. Turbulent flow is a fluid flow in which the velocity at a given point varies erratically in magnitude and direction. In some aspects, the fluid flow through the constriction is laminar flow. Laminar flow involves uninterrupted flow in a fluid near a solid boundary in which the direction of flow at every point remains constant. In some aspects, the fluid flow is turbulent flow after the cells pass through the constriction. The velocity at which the cells pass through the constrictions can be varied. In some aspects, the cells pass through the constrictions at a uniform cell speed. In some aspects, the cells pass through the constrictions at a fluctuating cell speed.

In some aspects, a combination treatment is used to deliver a payload, e.g., the methods described herein followed by exposure to an electric field downstream of the constriction. In some aspects, the cell is passed through an electric field generated by at least one electrode after passing through the constriction. In some aspects, the electric field assists in delivery of a payload to a second location inside the cell such as the cell nucleus. In some aspects, one or more electrodes are in proximity to the cell-deforming constriction to generate an electric field. In some aspects, the electric field is between about 0.1 kV/m to about 100 MV/m. In some aspects, an integrated circuit is used to provide an electrical signal to drive the electrodes. In some aspects, the cells are exposed to the electric field for a pulse width of between about 1 ns to about 1 s and a period of between about 100 ns to about 10 s.

### Methods of Conditioning

In some aspects, enhanced APCs provided herein are conditioned to further improve one or more properties of the cells. In some aspects, the enhanced APCs are conditioned subsequent to constriction mediated delivery. In some aspects, the adjuvant comprises a CpG oligodeoxynucleotide (ODN), LPS, IFN-α, STING agonists, RIG-I agonists, poly I. C, R837, R848, a TLR3 agonist, a TLR4 agonist or a TLR 9 agonist. In some aspects, the adjuvant is CpG ODN 2006 (also known as CpG 7909) (TCGTCGTTTTGTCGTTTTGTCGTT (SEQ ID NO: 23)). In some aspects, the adjuvant is CpG 7909. In some aspects, the adjuvant is a CpG 7909 oligodeoxynucleotide (ODN).

In some aspects, conditioning the enhanced APCs provided herein comprises incubating the enhanced APCs with the adjuvant for about 1 to about 24 hours. In some aspects, the enhanced APCs are incubated with the adjuvant for about 2 to about 10 hours. In some aspects, the enhanced APCs are incubated with the adjuvant for about 3 to about 6 hours. In some aspects, the enhanced APCs are incubated with the adjuvant for about 1 hour, about 2 hours, about 3 hours, about 3.5 hours, about 4 hours, about 4.5 hours, about 5 hours, about 5.5 hours, about 6 hours, about 8 hours, about 12 hours, about 16 hours, about 20 hours, or about 24 hours. In some aspects, the enhanced APCs are incubated with the adjuvant for about 4 hours. In some aspects, the enhanced APCs are incubated with the adjuvant for about 3 hours. In some aspects, the enhanced APCs are incubated with the adjuvant at about 37° C. for about 4 hours. In some aspects, the enhanced APCs are incubated with the adjuvant at about 37° C. for about 3 hours. In some aspects, the enhanced APCs are incubated with CpG 7909 for about 4 hours. In some aspects, the enhanced APCs are incubated with CpG 7909 for about 3 hours. In some aspects, the concentration of the adjuvant (e.g., CpG 7909) is about 0.20 mg/mL, about 0.25 mg/mL, about 0.30 mg/mL, about 0.35 mg/mL, about 0.40 mg/mL, about 0.45 mg/mL, or about 0.50 mg/mL, or any concentration therebetween. In some aspects, the enhanced APCs are incubated with the CpG 7909 at a concentration of about 0.35 mg/mL. In some aspects, the enhanced APCs are incubated with the CpG 7909 at a concentration of about 0.35 mg/mL and at about 37° C. for about 4 hours. In some aspects, the enhanced APCs are incubated with the CpG 7909 at a concentration of about 0.35 mg/mL and at about 37° C. for about 3 hours.

### EXAMPLES

Those skilled in the art will recognize that several aspects are possible within the scope and spirit of this disclosure. The disclosure will now be described in greater detail by reference to the following non-limiting examples. The following examples further illustrate the disclosure but, of course, should not be construed as in any way limiting its scope.

### Example 1. Development of Enhanced APCs Described Herein

Each of five different mRNAs (encoding lIPV-16 E6 protein, lIPV-17 E7 protein, CD86, membrane-bound IL-2, and membrane-bound IIL-12) were intracellularly delivered to PBMCs using squeeze processing. Table 2 (below) provides the sequences for the mRNAs.

TABLE 2
Exemplary mRNA Sequences

| | |
|---|---|
| E6 mRNA (SEQ ID | GGAAAUAAGAGAGAAAAGAAGAGUAAGAAGAAAUAUA |
| NO: 18) | AGAGCCACCAUGCACCAGAAACGGACCGCCAUGUUCCA |
| | GGAUCCUCAAGAGAGGCCCAGAAAGCUGCCUCAGCUGU |
| | GUACCGAGCUGCAGACCACCAUCCACGACAUCAUCCUG |
| | GAAUGCGUGUACUGCAAGCAGCAGCUCCUGCGGAGAGA |
| | GGUGUACGAUUUCGCCUUCCGGGACCUGUGCAUCGUGU |
| | ACAGAGAUGGCAACCCCUACGCCGUGUGCGACAAGUGC |
| | CUGAAGUUCUACAGCAAGAUCAGCGAGUACCGGCACUA |
| | CUGCUACAGCCUGUACGGCACCACACUGGAACAGCAGU |
| | ACAACAAGCCCCUGUGCGACCUGCUGAUCCGGUGCAUC |
| | AACUGCCAGAAACCUCUGUGCCCCGAGGAAAAGCAGCG |
| | GCACCUGGACAAGAAGCAGCGGUUCCACAACAUCAGAG |
| | GCCGGUGGACCGGCAGAUGCAUGAGCUGUUGUCGGAGC |
| | AGCAGAACCAGACGGGAAACCCAGCUGUGAGCUGCCUU |
| | CUGCGGGGCUUGCCUUCUGGCCAUGCCCUUCUUCUCUC |
| | CCUUGCACCUGUACCUCUUGGUCUUUGAAUAAAGCCUG |
| | AGUAGGAAGUCUAGAAAAAAAAAAAAAAAAAAAAAAA |
| | AAAAAAAAAAAAAAAAAAAAAAAAAAAAAAAAAAAAA |
| | AAAAAAAAAAAAAAAAAAAA |
| E7 mRNA (SEQ ID | GGAAAUAAGAGAGAAAAGAAGAGUAAGAAGAAAUAUA |
| NO: 19) | AGAGCCACCAUGCACGGCGAUACCCCUACACUGCACGA |
| | GUACAUGCUGGACCUGCAGCCUGAGACAACCGACCUGU |
| | ACUGCUACGAGCAGCUGAACGACAGCAGCGAGGAAGAG |
| | GACGAGAUUGACGGACCUGCCGGACAGGCCGAACCUGA |
| | UAGAGCCCACUACAAUAUCGUGACCUUCUGCUGCAAGU |
| | GCGACAGCACCCUGAGACUGUGUGUGCAGAGCACCCAC |
| | GUGGACAUCAGAACCCUGGAAGAUCUGCUGAUGGGCAC |
| | CCUGGGCAUCGUGUGCCCUAUCUGUAGCCAGAAGCCUU |
| | GAGCUGCCUUCUGCGGGGCUUGCCUUCUGGCCAUGCCC |
| | UUCUUCUCUCCCUUGCACCUGUACCUCUUGGUCUUUGA |
| | AUAAAGCCUGAGUAGGAAGUCUAGAAAAAAAAAAAAA |
| | AAAAAAAAAAAAAAAAAAAAAAAAAAAAAAAAAAAAA |
| | AAAAAAAAAAAAAAAAAAAAAAAAAAAAAA |
| CD86 mRNA (SEQ | GGAAAUAAGAGAGAAAAGAAGAGUAAGAAGAAAUAUA |
| ID NO: 20) | AGAGCCACCAUGGAUCCCCAGUGUACCAUGGGCCUGAG |
| | CAACAUCCUGUUCGUGAUGGCCUUUCUGCUGAGCGGAG |
| | CCGCUCCUCUGAAGAUCCAGGCCUACUUCAACGAGACA |
| | GCCGACCUGCCUUGCCAGUUCGCCAACAGCCAGAAUCA |
| | GAGCCUGAGCGAGCUGGUGGUGUUCUGGCAGGACCAAG |
| | AGAACCUGGUGCUGAACGAGGUGUACCUGGGCAAAGAA |
| | AAGUUCGACAGCGUGCACAGCAAGUACAUGGGCAGAAC |
| | CAGCUUCGACUCCGACAGCUGGACACUGAGACUGCACA |
| | ACCUGCAGAUCAAGGACAAGGGCCUGUACCAGUGCAUC |
| | AUCCACCACAAGAAACCCACCGGCAUGAUCAGAAUCCA |
| | CCAGAUGAACAGCGAGCUGAGCGUGCUGGCCAACUUCA |
| | GCCAGCCUGAGAUCGUGCCCAUCAGCAACAUCACCGAG |
| | AACGUGUACAUCAACCUGACCUGCAGCAGCAUCCACGG |
| | CUACCCCGAGCCUAAGAAAAUGUCCGUGCUGCUGCGGA |
| | CCAAGAACAGCACCAUCGAGUACGACGGCGUGAUGCAG |
| | AAAAGCCAGGACAACGUGACCGAGCUGUACGACGUGUC |
| | CAUCAGCCUGUCCGUGUCAUUCCCCGACGUGACCAGCA |
| | AUAUGACCAUCUUCUGCAUCCUGGAAACCGACAAGACC |
| | CGGCUGCUGUCUAGCCCCUUCAGCAUCGAGCUGGAAGA |
| | UCCCCAGCCUCCUCCUGAUCACAUCCCUUGGAUUACCGC |
| | CGUGCUGCCCACCGUGAUCAUCUGCGUGAUGGUGUUUU |
| | GCCUGAUCCUGUGGAAGUGGAAGAAGAAGAAACGGCCU |
| | CGGAACAGCUACAAGUGCGGCACCAACACCAUGGAACG |
| | GGAAGAGAGCGAGCAGACCAAGAAGAGAGAGAAGAUUC |
| | ACAUCCCCGAGCGGAGCGACGAAGCCCAGAGAGUGUUU |
| | AAGAGCAGCAAGACCAGCAGCUGCGACAAGAGCGAUAC |
| | CUGCUUCUGAGCUGCCUUCUGCGGGGCUUGCCUUCUGG |
| | CCAUGCCCUUCUUCUCUCCCUUGCACCUGUACCUCUUG |
| | GUCUUUGAAUAAAGCCUGAGUAGGAAGUCUAGAAAAAA |
| | AAAAAAAAAAAAAAAAAAAAAAAAAAAAAAAAAAAAA |
| | AAAAAAAAAAAAAAAAAAAAAAAAAAAAAAAAAAAAA |
| membrane-bound IL- | GGAAAUAAGAGAGAAAAGAAGAGUAAGAAGAAAUAUA |
| 2 mRNA (SEQ ID | AGAGCCACCAUGAUGGUGGACGGCGACAACAGCCACGU |
| NO: 21) | GGAAAUGAAGCUGGCCGUGGACGAGGAAGAGAACGCCG |
| | ACAACAACACCAAGGCCAACGUGACCAAGCCUAAGAGA |
| | UGCAGCGGCAGCAUCUGCUACGGCACAAUCGCCGUGAU |
| | CGUGUUCUUCCUGAUCGGCUUUAUGAUCGGCUACCUGG |
| | GCUACUGCAAGAGCAGUGAUGGACCUGGCGAAACAGGC |
| | GGAGGCGGAGGAUCUGGUGGCGGAGGAAGCGGUGGCGG |
| | CGGUUCUGCUCCUACAAGCAGCAGCACCAAGAAAACCC |
| | AGCUGCAACUGGAACAUCUGCUGCUGGACCUGCAGAUG |
| | AUCCUGAACGGCAUCAACAACUACAAGAACCCCAAGCU |
| | GACCCGGAUGCUGACCUUCAAGUUCUACAUGCCCAAGA |
| | AGGCCACCGAGCUGAAGCACCUCCAGUGCCUGGAAGAG |
| | GAACUGAAGCCCCUGGAAGAAGUGCUGAAUCUGGCCCA |
| | GAGCAAGAACUUCCACCUGAGGCCUAGGGACCUGAUCA |
| | GCAACAUCAAUGUGAUCGUGCUGGAACUCAAGGGCAGC |
| | GAGACAACCUUCAUGUGCGAGUACGCCGACGAGACAGC |
| | UACCAUCGUGGAAUUUCUGAACCGGUGGAUCACCUUCU |
| | GCCAGAGCAUCAUCAGCACCCUGACCUGAGCUGCCUUC |
| | UGCGGGGCUUGCCUUCUGGCCAUGCCCUUCUUCUCUCC |
| | CUUGCACCUGUACCUCUUGGUCUUUGAAUAAAGCCUGA |
| | GUAGGAAGUCUAGAAAAAAAAAAAAAAAAAAAAAAAA |
| | AAAAAAAAAAAAAAAAAAAAAAAAAAAAAAAAAAAAA |
| | AAAAAAAAAAAAAAAAAAA |
| membrane-bound IL- | GGAAAUAAGAGAGAAAAGAAGAGUAAGAAGAAAUAUA |
| 12 mRNA (SEQ ID | AGAGCCACCAUGAUGGUGGACGGCGACAACAGCCACGU |
| NO: 22) | GGAAAUGAAGCUGGCCGUGGACGAGGAAGAGAACGCCG |
| | ACAACAACACCAAGGCCAACGUGACCAAGCCUAAGAGA |
| | UGCAGCGGCAGCAUCUGCUACGGCACAAUCGCCGUGAU |
| | CGUGUUCUUCCUGAUCGGCUUUAUGAUCGGCUACCUGG |
| | GCUACUGCAAGAGCAGUGAUGGACCUGGCGAAACAGGC |
| | GGAGGCGGAGGAUCUGGUGGCGGAGGAAGUGGCGGCGG |
| | AGGUUCUAUUUGGGAGCUGAAGAAAGACGUGUACGUGG |
| | UGGAACUGGACUGGUAUCCCGAUGCUCCUGGCGAGAUG |
| | GUGGUGCUGACCUGCGAUACCCCUGAAGAGGACGGCAU |
| | CACCUGGACACUGGAUCAGUCUAGCGAGGUGCUCGGCA |
| | GCGGCAAGACCCUGACCAUCCAAGUGAAAGAGUUUGGC |
| | GACGCCGGCCAGUACACCUGUCACAAAGGCGGAGAAGU |
| | GCUGAGCCACAGCCUGCUGCUGCUCCACAAGAAAGAGG |
| | AUGGCAUUUGGAGCACCGACAUCCUGAAGGACCAGAAA |
| | GAGCCCAAGAACAAGACCUUCCUGAGAUGCGAGGCCAA |
| | GAACUACAGCGGCCGGUUCACAUGUUGGUGGCUGACCA |
| | CCAUCAGCACCGACCUGACCUUCAGCGUGAAGUCCAGC |
| | AGAGGCAGCAGUGAUCCUCAGGGCGUUACAUGUGGCGC |
| | CGCUACACUGUCUGCCGAAAGAGUGCGGGGCGAUAACA |
| | AAGAAUACGAGUACAGCGUGGAAUGCCAAGAGGACAGC |
| | GCCUGUCCAGCCGCCGAAGAGUCUCUGCCUAUCGAAGU |
| | GAUGGUCGACGCCGUGCACAAGCUGAAGUACGAGAACU |
| | ACACCAGCAGCUUUUUCAUCCGGGACAUCAUCAAGCCC |
| | GAUCCUCCAAAGAACCUGCAGCUGAAGCCUCUGAAGAA |
| | CAGCAGACAGGUGGAAGUGUCCUGGGAGUACCCCGACA |
| | CCUGGUCUACACCCCACAGCUACUUCAGCCUGACCUUU |
| | UGCGUGCAAGUGCAGGGCAAGUCCAAGCGCGAGAAAAA |
| | GGACCGGGUGUUCACCGACAAGACCAGCGCCACCGUGA |
| | UCUGCAGAAAGAACGCCAGCAUCAGCGUCAGAGCCCAG |
| | GACCGGUACUACAGCAGCUCUUGGAGCGAAUGGGCCAG |
| | CGUGCCAUGUAGCGGAGGUGGUGGUAGCGGAGGCGGCG |
| | GAAGCGGCGGUGGUGGAUCAGGUGGUGGUGGCUCUAGA |
| | AACCUGCCAGUGGCUACCCCUGAUCCUGGCAUGUUCCC |
| | UUGUCUGCACCACAGCCAGAACCUGCUGAGAGCCGUGU |
| | CCAACAUGCUGCAGAAGGCCAGACAGACCCUGGAAUUC |
| | UACCCCUGCACCAGCGAGGAAAUCGACCACGAGGACAU |
| | CACCAAGGAUAAGACCAGCACCGUGGAAGCCUGCCUGC |
| | CUCUGGAACUGACCAAGAACGAGAGCUGCCUGAACAGC |
| | CGGGAAACCUCCUUCAUCACCAACGGCUCUUGCCUGGC |
| | CAGCAGAAAGACAAGCUUCAUGAUGGCCCUGUGCCUGA |
| | GCAGCAUCUACGAGGACCUGAAGAUGUACCAGGUGGAA |
| | UUCAAGACCAUGAACGCCAAGCUGCUGAUGGACCCCAA |
| | GCGGCAGAUCUUCCUGGACCAGAACAUGCUGGCUGUGA |
| | UCGACGAGCUGAUGCAGGCCCUGAACUUCAACAGCGAG |
| | ACAGUGCCCCAGAAGUCUAGCCUGGAAGAACCCGACUU |
| | CUACAAGACCAAGAUCAAGCUGUGCAUCCUGCUGCACG |
| | CCUUCCGGAUCAGAGCCGUGACCAUCGACAGAGUGAUG |
| | AGCUACCUGAACGCCUCCUGAGCUGCCUUCUGCGGGGC |
| | UUGCCUUCUGGCCAUGCCCUUCUUCUCUCCCUUGCACC |
| | UGUACCUCUUGGUCUUUGAAUAAAGCCUGAGUAGGAAG |
| | UCUAGAAAAAAAAAAAAAAAAAAAAAAAAAAAAAAAA |
| | AAAAAAAAAAAAAAAAAAAAAAAAAAAAAAAAAAAAA |
| | AAAAAAAAAAA |

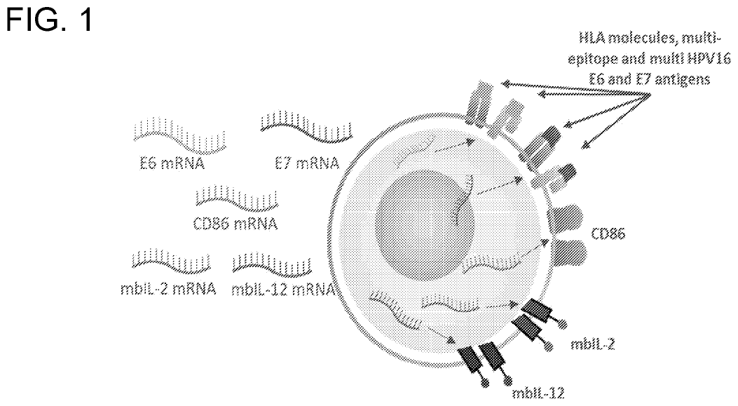

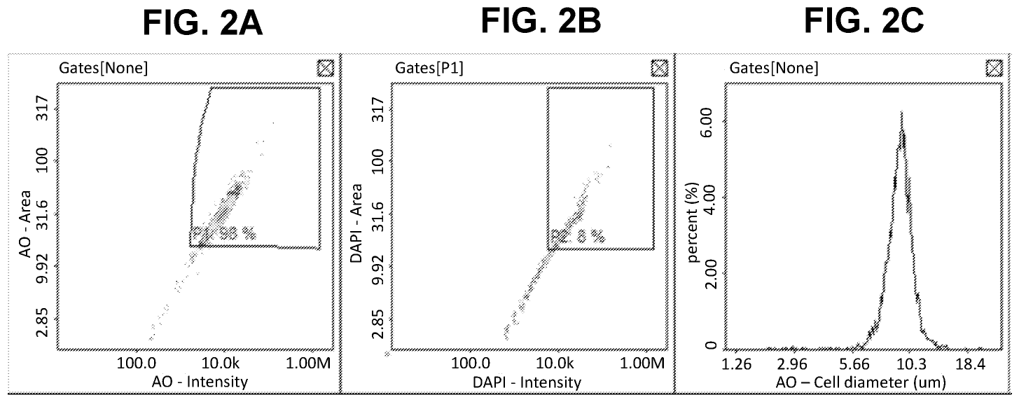

As illustrated in FIG. 1, upon entry into the PBMCs, the mRNAs are translated and the encoded proteins are expressed on the PBMCs to become the enhanced APCs described herein. After squeeze delivery, the viability of the enhanced APCs was assessed on the NC-200 cell counter, which provides the number of live cells/mL. Total cell count was derived from the number of events captured in the Acridine Orange positive (AO+) gate. Dead cell count was derived from the number of events captured in the DAPI positive (DAPI+) gate. AO+gate, DAPI and cell diameter are shown in FIGS. 2A-2C. Using such counts, the following formula was used to calculate the percent viability: (Total AO⁺ cells—Dead DAPI⁺ cells)/Total AO⁺ Cells×100. Exemplary results from the NC-200 cell counter are shown in Table 3 below.

TABLE 3
Viability
Gate Derived Results

| | |
|---|---|
| Viability (%) | 92.1 |
| Live (cells/mL) | 1.93 × 10\hat{ }6 |
| Dead (cells/mL) | 1.65 × 10\hat{ }5 |
| Total (cells/mL) | 2.10 × 10\hat{ }6 |

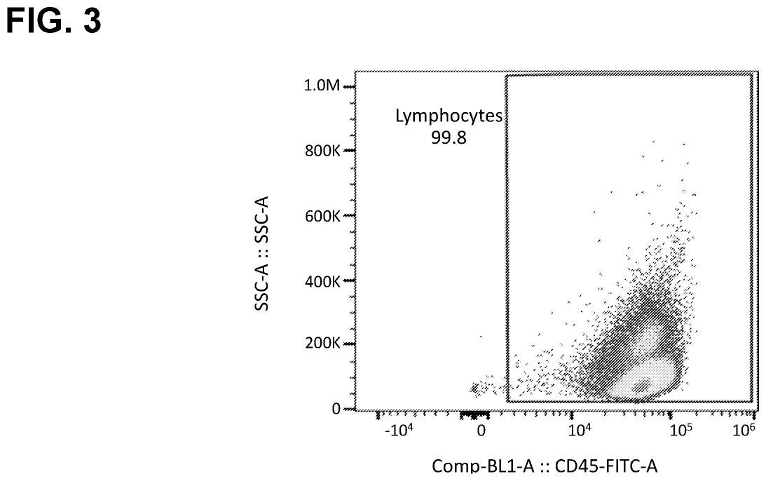

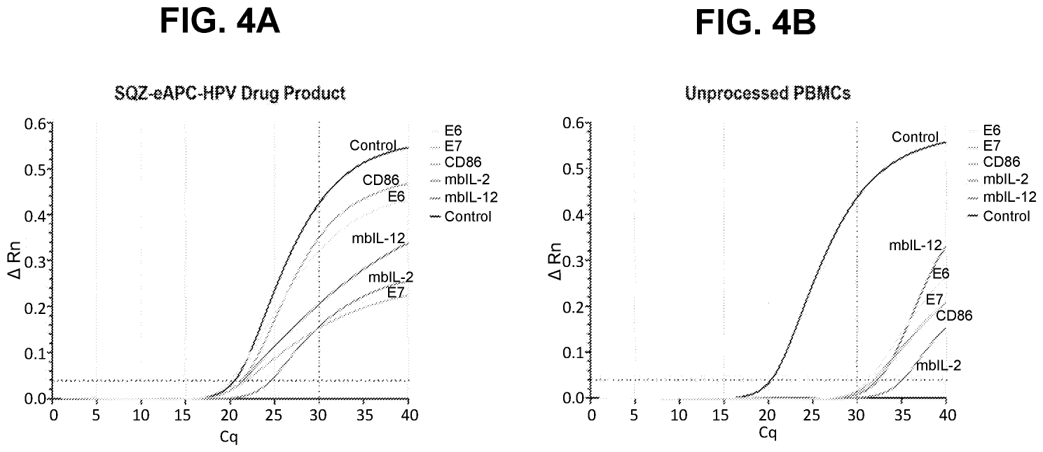

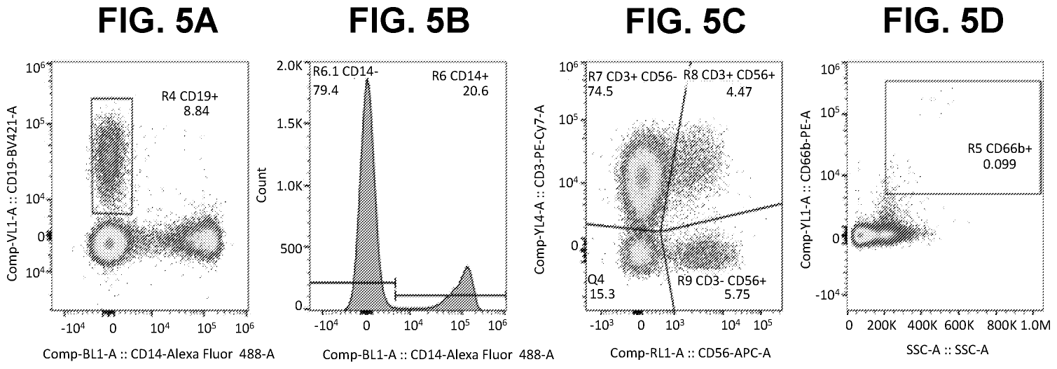

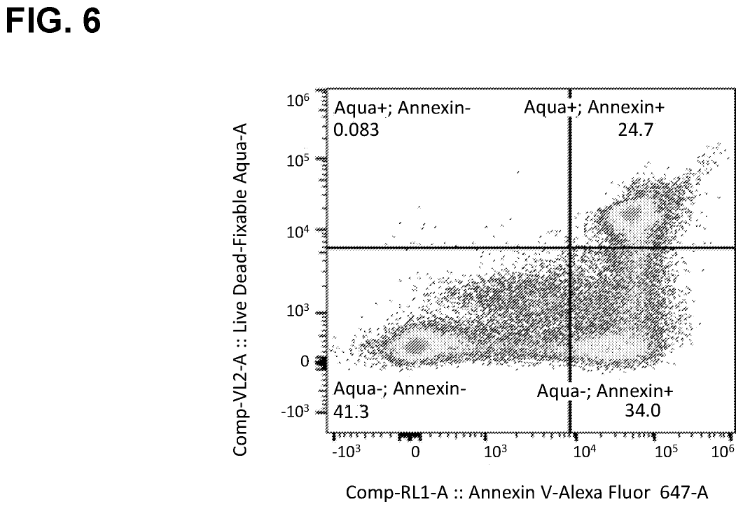

Next, a phenotypic analysis was performed using flow cytometry to assess the cellular make-up of the enhanced APCs. As shown in FIG. 3, nearly all of the enhanced APCs were positive for CD45 expression. Additionally, as shown in FIGS. 5A-5D, the enhanced APCs included primarily T cells, monocytes, B cells, and NK cells. No significant population of granulocytes were observed (see FIG. 5D). And, as shown in FIG. 6, a significant percentage of the enhanced APCs were positive for Annexin V staining. Although Annexin V positive cells are typically considered to be involved in the apoptosis pathway, here, it is believed that the positive staining was due to the transient membrane disruption that occurs with squeeze processing method described herein.

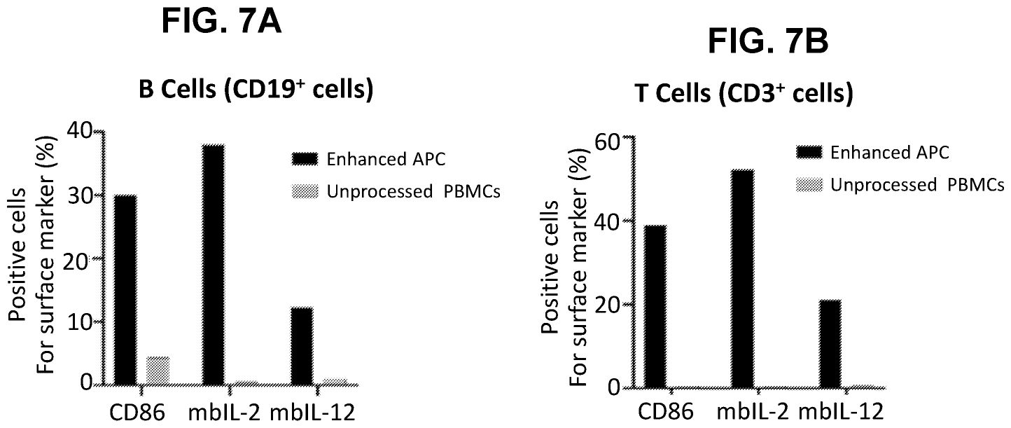

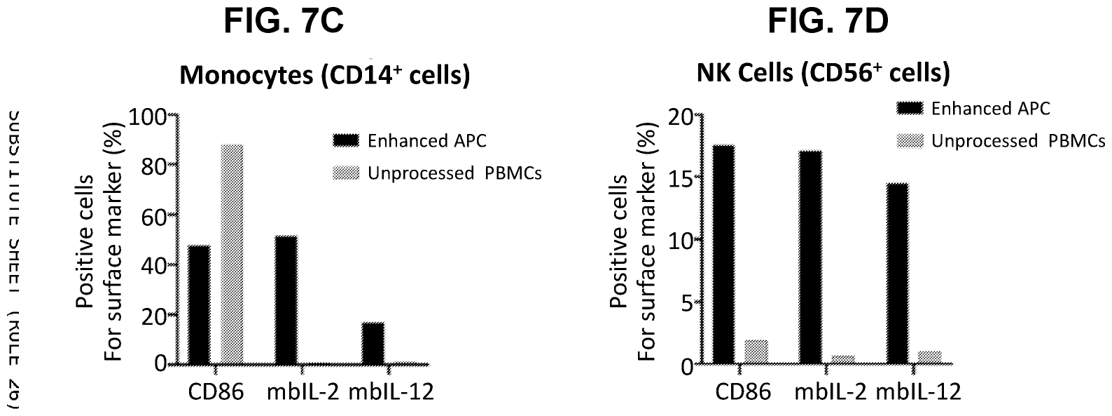

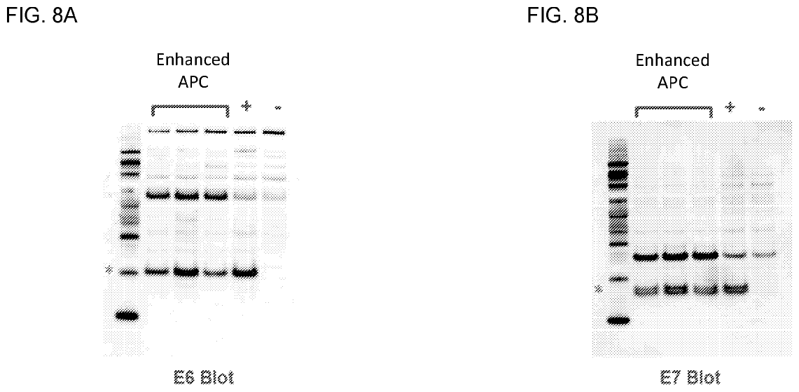

Next, the potency of the enhanced APCs was assessed by measuring the expression of the proteins that were encoded by the five mRNAs: HPV-16 E6 protein, HPV-16 E7 protein, CD86, membrane-bound IL-12, and membrane-bound IL-2. As shown in FIGS. 7A-7D, each of the cellular subtypes present within the enhanced APCs (i.e., B cells, T cells, monocytes, and NK cells) exhibited significantly higher expression of CD86, mbIL-2, and mbIL-12 as compared to the control unprocessed PBMCs. Additionally, using Western blot analysis, the enhanced APCs also expressed significantly higher levels of both the HPV-16 E6 protein and the HPV-16 E7 proteins (see FIGS. 8A and 8B).

Collectively, the above results confirm that the squeeze processing methods provided herein were able to successfully modify the PBMCs to produce the enhanced APCs of the present disclosure.

### Example 2. Development and Use of Formulations Described Herein

The pharmaceutical formulations described herein is useful as a sterile autologous cell therapy product, in which the cells are positive for the main cell type surface marker CD45 with cell viability of at least 70% (see Example 1). The pharmaceutical formulation was prepared by formulating the enhanced APCs (described in Example 1) in a solution containing 50% (w/w) of CryoStor® CS10, 30% (w/w) of HypoThermosol® FRS, and 20% (w/w) of 25% Human Serum Albumin (HSA) to a target concentration of 11×10⁶ live cells/mL, and filling into vials. The pH of the formulation was adjusted to 7.0-7.9. The vials were cryopreserved using a chamber temperature of ≤−170° C., with the vials reaching and maintaining a temperature of −140° C. during the different stages of the development process (e.g., production and storage). The fill volume (i.e., 9.5 mL) was selected as it takes into account the anticipated cell concentration post-thaw (8.5×10⁶ live cells/mL), and the extractable volume (8.9 mL), for a deliverable live cell dose per vial of 76×10⁶ live cells.

Table 4 provides additional description of the pharmaceutical formulation described herein.

TABLE 4

| | | | Targeted | | | | |
|---|---|---|---|---|---|---|---|
| | | | Deliverable | | | | |
| | Targeted | | Amount Per | | Target | Expected | |
| | Amount Per | | Vial Post- | | Concentration | Concentration | |
| Ingredient | Vial at Fill^{a} | | Thaw^{b,c} | | at Fill | Post-Thaw | Function |
| Drug substance | | | | | | | |
| SQZ-eAPC-HPV | 105 × 10⁶ | | 76 × 10⁶ | | 11 × 10⁶ | 8.5 × 10⁶ | Active |
| Drug Substance | live cells | | live cells | | live cells/mL | live cells/mL^{d} | |
| Excipient(s) | | | | | | | |
| CryoStor ® CS10 | 4.99 | g | 4.45 | mL | 50% (w/w) | 50% (w/w) | Cryoprotectant, |
| | | | | | | | Osmolality Agent, |
| | | | | | | | pH Control |
| HypoThermosol ® | 2.99 | g | 2.67 | mL | 30% (w/w) | 30% (w/w) | Osmolality Agent, |
| FRS | | | | | | | pH Control |
| 25% Human | 2.00 | g | 1.78 | mL | 20% (w/w) | 20% (w/w) | Stabilizer |
| Serum Albumin | | | | | | | |
| Total | 9.98 | g^{f} | 8.9 | mL | | | |

^{a}The targeted amount of live cells per vial at fill is the targeted concentration at fill multiplied by the fill volume of 9.5 mL. Target amounts of excipients at fill is equal to the total fill weight (9.98 g) multiplied by the percent (w/w) of each excipient.
^{b}Deliverable volume is based on using a AT-Adapt vial access device, which results in a maximum delivered volume of 8.9 mL.
^{c}Deliverable amount per vial post-thaw is the expected concentration post-thaw multiplied by the extractable volume ((8.5 × 10⁶ live cells/mL) × 8.9 mL).
^{d}Expected concentration post-thaw is the target concentration at fill multiplied by the observed recovery for the tech transfer batches for final drug product formulation and recovery post-thaw ((11 × 10⁶ live cells/mL) × 0.83 × 0.93).
^{e} There is no SQZ-eAPC-HPV drug substance reference standard, given the autologous cell nature of the product.
^{f}9.98 g at fill is equivalent to 9.5 mL at fill.

Table 5 provides a summary of the quality target product profile and appropriate testing measures for the pharmaceutical formulation described herein.

TABLE 5

| | | | Specification (acceptance criteria |
|---|---|---|---|
| Parameter | | Test Method | applied) |
| Appearance | | Visual | Off white, yellow, pink or red cell |
| | | | suspension free of visible particulates |
| Concentration^{a} | Total Cell Count | Cell counter | Report total cell number/mL |
| | Viability % | Cell counter | ≥70% |
| | Live Cell Count | Calculation | Report live cells/mL |

| Identity - Cell Composition | Flow cytometer | Positive for main cell type surface |
|---|---|---|
| and Frequency | | marker CD45 ≥95% CD45⁺ cells |
| Identity - mRNA | qPCR | Presence of CD86 mRNA, mbIL-2 |
| | | mRNA, mbIL-12 mRNA, E6 mRNA |
| | | and E7 mRNA |
| Potency - mRNA Content | qPCR | Report Cq value for CD86 mRNA, |
| | | mbIL-2 mRNA, mbIL-12 mRNA, E6 |
| | | mRNA and E7 mRNA |
| Product Related Impurities | Flow Cytometer | ≤10% CD41a⁺ cells relative to the sum |
| (Platelets) | | of CD45⁺ cells, CD235a⁺ cells and |
| | | CD41a⁺ cells |
| pH | USP<791> | 7.0-7.9 |
| Endotoxin | USP<85>, Endosafe PTS ™ Kinetic | ≤4.2 EU/mL^{b} |
| | Chromogenic LAL Assay | |
| Mycoplasma | USP <1223>/21 CFR 610.9, | Not present^{c} |
| | MycoSEQ ™ real-time PCR | |
| | (Performed on in-process cell | |
| | washes) | |
| Sterility | 21 CFR 610.12, Milliflex ® Rapid, | No growth |
| | ATP bioluminescence | |

Table 6 provides the results of an exemplary stability study for the pharmaceutical formulation of the present disclosure.

TABLE 6

| | | | T = 0 | T = 1 Month | T = 2 Month |
|---|---|---|---|---|---|
| Test | Current Criteria | | (Release) | (26 Oct. 2021) | (2 Dec. 2021) |
| Appearance | Off white, yellow, pink, or red cell | | Off white cell | Off white cell | Off white cell |
| | suspension free of visible particulates | | suspension free | suspension free | suspension free |
| | | | of visible | of visible | of visible |
| | | | particulates | particulates | particulates |
| Concentration | Total Cell Count | Report Total | 1.21 × 10⁷ | 1.13 × 10⁷ | 1.15 × 10⁷ |
| | | Cell Number | | | |
| | | in cells/mL | | | |
| | Viability % | ≥70% | 89 | 86 | 84 |
| | Live Cell Count | Report live | 1.07 × 10⁷ | 9.64 × 10⁶ | 9.65 × 10⁶ |
| | | cells/mL | | | |
| Identity - Cell | Positive for main cell type surface | | Positive | NA | NA |
| Composition | marker CD45 | | | | |
| and Frequency | ≥95% CD45⁺ cells | | 100 | NA | NA |
| Identity - | Presence of CD86 | CD86 | Present | NA | NA |
| mRNA | mRNA, mbIL-2 | mbIL-2 | Present | NA | NA |
| | mRNA, mbIL-12 | mbIL-12 | Present | NA | NA |
| | mRNA, E6 | E6 | Present | NA | NA |
| | mRNA and E7 | E7 | Present | NA | NA |
| | mRNA | | | | |
| Potency - | Report Cq value | CD86 | 20.99 | 21.04 | 20.26 |
| mRNA Content | for CD86 mRNA, | mbIL-2 | 20.91 | 21.16 | 20.19 |
| | mbIL-2 mRNA, | mbIL-12 | 24.94 | 24.69 | 23.56 |
| | mbIL-12 mRNA, | E6 | 18.97 | 19.04 | 18.35 |
| | E6 mRNA and E7 | E7 | 21.45 | 21.65 | 21.00 |
| | mRNA | | | | |
| Product Related | ≤10% CD41a⁺ cells relative to the | | 2 | NA | NA |
| Impurities - | sum of CD45⁺ cells, CD235a⁺ cells | | | | |
| Platelets | and CD41a⁺ cells | | | | |
| pH | 7.0-7.9 | | 7.5 | NA | NA |
| Endotoxin | ≤4.2 EU/mL | | <0.5 | NA | NA |
| Sterility | No growth | | No growth | NA | NA |
| (Milliflex ® | | | | | |
| Rapid)^{d} | | | | | |

TABLE 7
Exemplary Sequences

| | | |
|---|---|---|
| 1 | ATGATGGTGGACGGCGACAACAGCCACGTGGAAATGAAGCTG | TFRC_G4S_IFN-a2a |
| | GCCGTGGACGAGGAAGAGAACGCCGACAACAACACCAAGGCC | mRNA |
| | AACGTGACCAAGCCTAAGAGATGCAGCGGCAGCATCTGCTAC | |
| | GGCACAATCGCCGTGATCGTGTTCTTCCTGATCGGCTTTATGAT | |
| | CGGCTACCTGGGCTACTGCAAGAGCAGTGATGGACCTGGCGAA | |
| | ACAGGCGGAGGCGGAGGATCTGGTGGCGGAGGAAGCGGTGGC | |
| | GGCGGATCTTGTGATCTGCCTCAGACACACAGCCTGGGCAGCA | |
| | GACGAACACTGATGCTGCTGGCCCAGATGCGGAAGATCAGCCT | |
| | GTTCAGCTGCCTGAAGGACCGGCACGATTTCGGCTTCCCTCAA | |
| | GAGGAATTCGGCAACCAGTTCCAGAAGGCCGAGACAATCCCT | |
| | GTGCTGCACGAGATGATCCAGCAGATCTTCAACCTGTTCTCCA | |
| | CCAAGGACAGCAGCGCCGCCTGGGATGAGACACTGCTGGACA | |
| | AGTTCTACACCGAGCTGTACCAGCAGCTGAATGACCTGGAAGC | |
| | CTGCGTGATCCAAGGCGTGGGAGTGACAGAGACACCCCTGATG | |
| | AAGGAAGATAGCATCCTGGCCGTGCGCAAGTACTTCCAGCGGA | |
| | TCACCCTGTACCTGAAAGAGAAGAAGTACAGCCCCTGCGCCTG | |
| | GGAAGTCGTGCGGGCCGAAATCATGAGAAGCTTCAGCCTGAG | |
| | CACCAACCTGCAAGAGAGCCTGCGGAGCAAAGAGTGA | |
| 2 | ATGATGGTGGACGGCGACAACAGCCACGTGGAAATGAAGCTG | TFRC_G4S_IL-12 |
| | GCCGTGGACGAGGAAGAGAACGCCGACAACAACACCAAGGCC | mRNA |
| | AACGTGACCAAGCCTAAGAGATGCAGCGGCAGCATCTGCTAC | |
| | GGCACAATCGCCGTGATCGTGTTCTTCCTGATCGGCTTTATGAT | |
| | CGGCTACCTGGGCTACTGCAAGAGCAGTGATGGACCTGGCGAA | |
| | ACAGGCGGAGGCGGAGGATCTGGTGGCGGAGGAAGTGGCGGC | |
| | GGAGGTTCTATTTGGGAGCTGAAGAAAGACGTGTACGTGGTGG | |
| | AACTGGACTGGTATCCCGATGCTCCTGGCGAGATGGTGGTGCT | |
| | GACCTGCGATACCCCTGAAGAGGACGGCATCACCTGGACACTG | |
| | GATCAGTCTAGCGAGGTGCTCGGCAGCGGCAAGACCCTGACCA | |
| | TCCAAGTGAAAGAGTTTGGCGACGCCGGCCAGTACACCTGTCA | |
| | CAAAGGCGGAGAAGTGCTGAGCCACAGCCTGCTGCTGCTCCAC | |
| | AAGAAAGAGGATGGCATTTGGAGCACCGACATCCTGAAGGAC | |
| | CAGAAAGAGCCCAAGAACAAGACCTTCCTGAGATGCGAGGCC | |
| | AAGAACTACAGCGGCCGGTTCACATGTTGGTGGCTGACCACCA | |
| | TCAGCACCGACCTGACCTTCAGCGTGAAGTCCAGCAGAGGCAG | |
| | CAGTGATCCTCAGGGCGTTACATGTGGCGCCGCTACACTGTCT | |
| | GCCGAAAGAGTGCGGGGCGATAACAAAGAATACGAGTACAGC | |
| | GTGGAATGCCAAGAGGACAGCGCCTGTCCAGCCGCCGAAGAG | |
| | TCTCTGCCTATCGAAGTGATGGTCGACGCCGTGCACAAGCTGA | |
| | AGTACGAGAACTACACCAGCAGCTTTTTCATCCGGGACATCAT | |
| | CAAGCCCGATCCTCCAAAGAACCTGCAGCTGAAGCCTCTGAAG | |
| | AACAGCAGACAGGTGGAAGTGTCCTGGGAGTACCCCGACACC | |
| | TGGTCTACACCCCACAGCTACTTCAGCCTGACCTTTTGCGTGCA | |
| | AGTGCAGGGCAAGTCCAAGCGCGAGAAAAAGGACCGGGTGTT | |
| | CACCGACAAGACCAGCGCCACCGTGATCTGCAGAAAGAACGC | |
| | CAGCATCAGCGTCAGAGCCCAGGACCGGTACTACAGCAGCTCT | |
| | TGGAGCGAATGGGCCAGCGTGCCATGTAGCGGAGGTGGTGGT | |
| | AGCGGAGGCGGCGGAAGCGGCGGTGGTGGATCAGGTGGTGGT | |
| | GGCTCTAGAAACCTGCCAGTGGCTACCCCTGATCCTGGCATGT | |
| | TCCCTTGTCTGCACCACAGCCAGAACCTGCTGAGAGCCGTGTC | |
| | CAACATGCTGCAGAAGGCCAGACAGACCCTGGAATTCTACCCC | |
| | TGCACCAGCGAGGAAATCGACCACGAGGACATCACCAAGGAT | |
| | AAGACCAGCACCGTGGAAGCCTGCCTGCCTCTGGAACTGACCA | |
| | AGAACGAGAGCTGCCTGAACAGCCGGGAAACCTCCTTCATCAC | |
| | CAACGGCTCTTGCCTGGCCAGCAGAAAGACAAGCTTCATGATG | |
| | GCCCTGTGCCTGAGCAGCATCTACGAGGACCTGAAGATGTACC | |
| | AGGTGGAATTCAAGACCATGAACGCCAAGCTGCTGATGGACCC | |
| | CAAGCGGCAGATCTTCCTGGACCAGAACATGCTGGCTGTGATC | |
| | GACGAGCTGATGCAGGCCCTGAACTTCAACAGCGAGACAGTG | |
| | CCCCAGAAGTCTAGCCTGGAAGAACCCGACTTCTACAAGACCA | |
| | AGATCAAGCTGTGCATCCTGCTGCACGCCTTCCGGATCAGAGC | |
| | CGTGACCATCGACAGAGTGATGAGCTACCTGAACGCCTCCTGA | |
| 3 | (G₄S)₃ | G₄S Linker |
| 4 | (EAAAK)₃ | EAAAK linker |
| 5 | (G₄S)ₙ | G4S Linker |
| 6 | (EAAAK)ₙ | EAAAK linker |
| 7 | MMVDGDNSHVEMKLAVDEEENADNNTKANVTKPKRCSGSICYG | TFRC_G4S_IL-2 |
| | TIAVIVFFLIGFMIGYLGYCKSSDGPGETGGGGGSGGGGSGGGGS | |
| | APTSSSTKKTQLQLEHLLLDLQMILNGINNYKNPKLTRMLTFKFY | |
| | MPKKATELKHLQCLEEELKPLEEVLNLAQSKNFHLRPRDLISNINV | |
| | IVLELKGSETTFMCEYADETATIVEFLNRWITFCQSIISTL | |
| 8 | MLKKRGNHSTGLCLLVMFFMVLVALVGLGLGMFQLFHLQKETG | FasL_G4S_IL-2 |
| | GGGGSGGGGSGGGGS | |
| | APTSSSTKKTQLQLEHLLLDLQMILNGINNYKNPKLTRMLTFKFY | |
| | MPKKATELKHLQCLEEELKPLEEVLNLAQSKNFHLRPRDLISNINV | |
| | IVLELKGSETTFMCEYADETATIVEFLNRWITFCQSIISTLT | |
| 9 | MMVDGDNSHVEMKLAVDEEENADNNTKANVTKPKRCSGSICYG | TFRC_G4S_IL-12 |
| | TIAVIVFFLIGFMIGYLGYCKSSDGPGETGGGGGSGGGGSGGGGS | |
| | IWELKKDVYVVELDWYPDAPGEMVVLTCDTPEEDGITWTLDQSS | |
| | EVLGSGKTLTIQVKEFGDAGQYTCHKGGEVLSHSLLLLHKKEDGI | |
| | WSTDILKDQKEPKNKTFLRCEAKNYSGRFTCWWLTTISTDLTFSV | |
| | KSSRGSSDPQGVTCGAATLSAERVRGDNKEYEYSVECQEDSACP | |
| | AAEESLPIEVMVDAVHKLKYENYTSSFFIRDIIKPDPPKNLQLKPL | |
| | KNSRQVEVSWEYPDTWSTPHSYFSLTFCVQVQGKSKREKKDRVF | |
| | TDKTSATVICRKNASISVRAQDRYYSSSWSEWASVPCSGGGGSGG | |
| | GGSGGGGSGGGGSRNLPVATPDPGMFPCLHHSQNLLRAVSNMLQ | |
| | KARQTLEFYPCTSEEIDHEDITKDKTSTVEACLPLELTKNESCLNS | |
| | RETSFITNGSCLASRKTSFMMALCLSSIYEDLKMYQVEFKTMNAK | |
| | LLMDPKRQIFLDQNMLAVIDELMQALNFNSETVPQKSSLEEPDFY | |
| | KTKIKLCILLHAFRIRAVTIDRVMSYLNAS | |
| 10 | MMVDGDNSHVEMKLAVDEEENADNNTKANVTKPKRCSGSICYG | TFRC_G4S_IFN-a2a |
| | TIAVIVFFLIGFMIGYLGYCKSSDGPGETGGGGGSGGGGSGGGGS | |
| | CDLPQTHSLGSRRTLMLLAQMRKISLFSCLKDRHDFGFPQEEFGN | |
| | QFQKAETIPVLHEMIQQIFNLFSTKDSSAAWDETLLDKFYTELYQQ | |
| | LNDLEACVIQGVGVTETPLMKEDSILAVRKYFQRITLYLKEKKYS | |
| | PCAWEVVRAEIMRSFSLSTNLQESLRSKE | |
| 11 | MMVDGDNSHVEMKLAVDEEENADNNTKANVTKPKRCSGSICYG | TFRC domain |
| | TIAVIVFFLIGFMIGYLGYCKSSDGPGETG | |
| 12 | MLKKRGNHSTGLCLLVMFFMVLVALVGLGLGMFQLFHLQKETG | FasL domain |
| 13 | ATGATGGTGGACGGCGACAACAGCCACGTGGAGATGAAGCTG | membrane-bound IL-7 |
| | GCCGTGGACGAGGAGGAGAACGCCGACAACAACACCAAGGCC | mRNA |
| | AACGTGACCAAGCCCAAGCGCTGCAGCGGCAGCATCTGCTACG | |
| | GCACCATCGCCGTGATCGTGTTCTTCCTGATCGGCTTCATGATC | |
| | GGCTACCTGGGCTACTGCAAGAGCAGCGACGGCCCCGGCGAG | |
| | ACCGGCGGaGGCGGCGGaAGCGGCGGCGGaGGCAGCGGCGGaG | |
| | GCGGaAGCGACTGCGACATCGAGGGCAAGGACGGCAAGCAGT | |
| | ACGAGAGCGTGCTGATGGTGAGCATCGACCAGCTGCTGGACA | |
| | GCATGAAGGAGATCGGCAGCAACTGCCTGAACAACGAGTTCA | |
| | ACTTCTTCAAGCGCCACATCTGCGACGCCAACAAGGAGGGCAT | |
| | GTTCCTGTTCCGCGCCGCCCGCAAGCTGCGCCAGTTCCTGAAG | |
| | ATGAACAGCACCGGCGACTTCGACCTGCACCTGCTGAAGGTGA | |
| | GCGAGGGCACCACCATCCTGCTGAACTGCACCGGCCAGGTGAA | |
| | GGGCCGCAAGCCCGCCGCCCTGGGCGAGGCCCAGCCCACCAA | |
| | GAGCCTGGAGGAGAACAAGAGCCTGAAGGAGCAGAAGAAGCT | |
| | GAACGACCTGTGCTTCCTGAAGCGCCTGCTGCAGGAGATCAAG | |
| | ACCTGCTGGAACAAGATCCTGATGGGCACCAAGGAGCACtaa | |

1. (canceled)

2. A pharmaceutical formulation comprising: (a) enhanced antigen presenting cells (“enhanced APCs”), (b) a cryopreservation medium, (c) a hypothermic preservation medium, and (d) a solution comprising human serum albumin (“human serum albumin solution”), wherein the enhanced APCs are capable of activating T cells in an HLA agnostic manner.

3. The formulation of claim 2, wherein:
(i) the human serum albumin solution comprises 25% human serum albumin and the human serum albumin solution is at a concentration of about 15% to about 25% (w/w);
(ii) the cryopreservation medium is at a concentration of about 40% to about 95% (w/w);
(iii) the hypothermic preservation medium is at a concentration of about 25% to about 35% (w/w); and/or
(iv) the pH of the formulation is about 6.0 to about 8.5.

4. The formulation of claim 2, which comprises:
(i) about 5×10⁶ enhanced APCs to about 1×10⁹ enhanced APCs;
(ii) about 1.05×10⁸ enhanced APCs;
(iii) about 1×10⁴ enhanced APCs/mL to about 1×10⁹ enhanced APCs/mL;
(iv) about 1×10⁶ enhanced APCs/mL to about 1×10⁸ enhanced APCs/mL; or
(v) about 1.1×10⁷ enhanced APCs/mL.

5-8. (canceled)

9. The formulation of claim 2, wherein the viability of the enhanced APCs is at least about 70%, at least about 80%, at least about 90%, or about 100% and wherein the enhanced APCs in the formulation maintain about ≥70% viability following storage for at least about 12 months at ≤−140° C.

10-21. (canceled)

22. A pharmaceutical formulation comprising: (a) enhanced antigen present cells (“enhanced APCs”) at a concentration of about 1×10⁴ enhanced APCs/mL to about 1×10⁹ enhanced APCs/mL; (b) a cryopreservation medium at a concentration of about 40% (w/w) to about 95% (w/w); (c) a hypothermic preservation medium at a concentration of about 25% (w/w) to about 35% (w/w); (d) a solution comprising about 25% human serum albumin (“human serum albumin solution”) at a concentration of about 15% (w/w) to about 25% (w/w); wherein the pH of the formulation is about 6.0 to about 8.5, and wherein the enhanced APCs are capable of activating T cells in an HLA agnostic manner.

23-26. (canceled)

27. The formulation of claim 2, wherein the cryopreservation medium is CryoStor® CS10 and/or the hypothermic preservation medium is HypoThermasol® FRS.

28-32. (canceled)

33. The formulation of claim 2, wherein the enhanced APCs comprise T cells, B cells, NK cells, monocytes, or combinations thereof.

34-39. (canceled)

40. The formulation of claim 2, wherein the enhanced APCs exhibit increased expression of a co-stimulatory molecule or a cytokine as compared to corresponding non-enhanced APCs (“reference APCs”).

41. The formulation of claim 40, wherein the co-stimulatory molecule comprises CD86 and wherein the cytokine comprises a membrane-bound cytokine.

42-43. (canceled)

44. The formulation of claim 40, wherein the cytokine comprises IL-2, IL-12, or both.

45. The formulation of claim 2, wherein the enhanced APCs have been prepared by passing a cell suspension comprising input APCs through a cell-deforming constriction, thereby causing perturbations in the APCs such that a nucleic acid encoding an antigen, a nucleic acid encoding a co-stimulatory molecule and/or a nucleic acid encoding a cytokine enters the APCs through the perturbations when contacted with the APCs, and thereby producing the enhanced APCs.

46. (canceled)

47. The formulation of claim 5, wherein the nucleic acid encoding the antigen, the nucleic acid encoding the co-stimulatory molecule, and/or the nucleic acid encoding the cytokine is a mRNA.

48. (canceled)

49. The formulation of claim 2, wherein the enhanced APCs have been conditioned in a medium comprising an adjuvant.

50. The formulation of claim 2, wherein the enhanced APCs have been conditioned in a medium comprising an adjuvant:
(i) for about 2 hours to about 10 hours;
(ii) for about 3 hours to about 6 hours;
(iii) for about 4 hours; or
(iv) at about 37° C.

51-61. (canceled)

62. A method of producing a formulation comprising enhanced antigen presenting cells (“enhanced APCs”) which are capable of activating T cells in an HLA agonostic manner, the method comprising combining the enhanced APCs, a cryopreservation medium, a hypothermic preservation medium, and a human serum albumin.

63. (canceled)

64. The method of claim 62, wherein after the combining, the formulation comprises:
(i) (a) about 5×10⁶ enhanced APCs to about 1×10⁹ enhanced APCs; (b) the cryopreservation medium at a concentration of about 40% (w/w) to about 95% (w/w); (c) the hypothermic preservation medium at a concentration of about 25% (w/w) to about 35% (w/w); and (d) the human serum albumin solution at a concentration of about 15% (w/w) to about 25% (w/w); and wherein the pH of the formulation is about 6.0 to about 8.5;
(ii) (a) about 1.1×10⁷ enhanced APCs/mL; (b) the cryopreservation medium at a concentration of about 50% (w/w); and (c) the human serum albumin solution at a concentration of about 20% (w/w); and wherein the formulation has a pH of about 7.0 to about 7.9; or
(iii) (a) about 1.05×10⁸ enhanced APCs, (b) about 4.99 q of the cryopreservation medium, and (c) about 2.00 q of the human serum albumin solution; and wherein the pH of the formulation is about 7.0 to about 7.9.

65-68. (canceled)

69. The method of claim 62, comprising passing a cell suspension which comprises input APCs through a cell-deforming constriction, thereby causing perturbations in the APCs such that a nucleic acid encoding an antigen, a nucleic acid encoding a co-stimulatory molecule, and/or a nucleic acid encoding a cytokine enters the input APCs through the perturbations when contacted with the APCs, and thereby producing the enhanced APCs.

70. (canceled)

71. The method of claim 69, wherein the nucleic acid encoding an antigen, the nucleic acid encoding a co-stimulatory molecule, and/or the nucleic acid encoding a cytokine is a mRNA.

72-77. (canceled)

78. The method of claim 69, wherein the co-stimulatory molecule comprises CD86 and wherein the cytokine comprises a membrane-bound cytokine.

79. (canceled)

80. The method of claim 69, wherein the cytokine comprises IL-2, IL-12, or both.
